(19) United States
(12) Patent Application Publication
(10) Pub. No.: US 2021/0308673 A1
(43) Pub. Date: Oct. 7, 2021

(54) Portable Diagnostic Apparatus and the Method Thereof

(71) Applicant: Sanwa Biotech Ltd, Hong Kong (CN)

(72) Inventors: Kelvin Chiu, Hong Kong (CN); Wai Lam William Yim, Hong Kong (CN); Isabelle Cecile Angele Dutry, Hong Kong (CN)

(73) Assignee: Sanwa Biotech Ltd, Hong Kong (CN)

(21) Appl. No.: 17/269,957

(22) PCT Filed: Aug. 23, 2019
(86) PCT No.: PCT/CN2019/102233

§ 371 Date: Feb. 19, 2021

Related U.S. Application Data
(60) Provisional application No. 62/722,174, filed on Aug. 23, 2018.

(51) Int. Cl. B01L 3/00; B01L 7/00; G01N 33/543; G16H 40/67

(52) U.S. Cl. CPC B01L 3/502715; B01L 3/502746; B01L 7/00; G01N 33/54386; G16H 40/67; B01L 2200/10; B01L 2400/0677; B01L 2300/0663; B01L 2300/021; B01L 2200/04; B01L 2300/0883; B01L 2300/0887; B01L 2300/161; B01L 2300/1872

(57) ABSTRACT

A method and a portable diagnostic apparatus (20) for detecting at least one analyte from a sample using a microfluidic cartridge (22). The portable diagnostic apparatus (20) comprises a cartridge receiving unit, a cartridge driver unit (30) and an optical unit (32). A method and an apparatus of obtaining disease prevalence information comprising at least one of the portable diagnostic apparatus (20). A method and a system for managing a network of portable diagnostic apparatuses and obtaining disease prevalence information comprising at least one of the portable diagnostic apparatus (20). A diagnostic system with multiple automated features that is capable of providing a one-step solution to near-patient clinical evaluation and diagnosis.

### CROSS-REFERENCE TO RELATED APPLICATION

This application claims benefit under 35 U.S.C. § 119(e) of U.S. Provisional Application having Ser. No. 62,722,174 filed Aug. 23, 2018, which is hereby incorporated by reference herein in its entirety.

This application is related to PCT Application No PCT/CN2015/0700567, filed Aug. 5, 2015, the content of which is incorporated herein by reference in its entirety.

### FIELD OF TECHNOLOGY

This invention relates to a system for detecting analyte and the method of use thereof. More particularly, the present invention relates to a microfluidic cartridge, an apparatus for such microfluidic cartridge.

### BACKGROUND

Traditional diagnostic, screening, disease staging, veterinary, drug tests, etc. are often done in laboratories and testing is generally time-consuming, expensive and required many resources, supplies and support. Current systems require multiple steps between the initial sample collection and receipt of the diagnostic results. They require a high level of human involvement and thus are prone to human errors. In some models, reagents are added manually by the user, which means potential spillage of reagents could result in health and safety risks to the user. In certain models, multiple manual steps such as loading reagents are involved in a diagnostic test. Finally, in some models the results are interpreted by users, which may cause variability and even potential misinterpretation.

### SUMMARY

In the light of the foregoing background, it is an object of the present invention to provide an improved diagnostic system for detecting one or more analyte(s).

According to one aspect, the diagnostic system includes two parts: an apparatus for detecting at least one or more analyte(s) and a microfluidic cartridge.

In some embodiments, the diagnostic system is a portable and self-contained system for detecting analyte. In some embodiments, the diagnostic system performs at least one immunoassay. In some embodiments, the diagnostic system performs at least one immunofluorescence assay. In some embodiments, the apparatus comprises a microfluidic cartridge driver unit, an optical inspection unit, and a control unit and a power supply unit. In some embodiments, the apparatus can run the binding and detection of an analyte without any fluidic interfaces to the instrument. In some embodiments, the microfluidic cartridge receiving unit receives a microfluidic cartridge that holds a microarray and an integrated microfluidic chip. In some embodiments, the microfluidic cartridge receives a sample containing the analyte and performs different process steps in the detection of the analyte. In some embodiments, all of the process steps, including reaction of the analyte on the microarray, detection of the signal, analysis of the data, and display of the results, are performed automatically on a single tray by the apparatus without any user intervention. A complete detection of analyte using the invention takes only a few minutes.

In one aspect, provided is a portable diagnostic apparatus for detecting at least one analyte from a sample using a microfluidic cartridge. The microfluidic cartridge has a plurality of micropumps, a plurality of reservoirs connected to at least one diagnostic portion via microchannels and a plurality of microvalves for sealing fluids in the reservoirs from flowing into the reaction site. The portable diagnostic apparatus includes a cartridge receiving unit, a cartridge driver unit and an optical unit. The cartridge receiving unit is configured to receive the microfluidic cartridge. The cartridge driver unit includes a) a microvalve controller configured to control the microvalves, and b) a micropump controller configured to actuate the micropumps. The micropump controller and the microvalve controller may cooperatively operate to actuate the flow of fluids from the reservoirs to the diagnostic portion in a predetermined sequence when the microfluidic cartridge is placed into the cartridge receiving unit. The optical unit is aligned with the diagnostic portion when the microfluidic cartridge is placed into the cartridge receiving unit. The portable diagnostic apparatus can control and monitor reactions within the microfluidic cartridge.

In some embodiments, the microvalve controller comprises at least one heating element configured to apply heat energy to a heat-deformable surface of the at least one microvalve to cause the microvalve to open.

In some embodiments, the at least one heating element is juxtapose to at least one microvalve of the microfluidic cartridge when the cartridge is placed into the cartridge receiving unit.

In some embodiments, the heating element is an infra-red emitter.

In some embodiments, the micropump controller comprises at least one electrical connector for electrical connection with the at least one micropump of the microfluidic cartridge and is configured to provide electrical current to the at least one micropump.

In some embodiments, the optical unit comprises an illumination component and a sensor component. The illumination component is configured to deliver light to a diagnostic portion of the microfluidic cartridge. The sensor component is configured to detect at least one signal generated from the diagnostic portion cause by the presence of an analyte when a microfluidic cartridge is inserted and operated at a predetermined condition.

In some embodiments, the illumination component comprises a light source having a wavelength in the range of 600 nm to 650 nm and the at least one data signal is a fluorescent signal.

In some embodiments, the portable diagnostic apparatus further comprises a control unit configured to perform one or more of the following: a. provide a predetermined sequence to the cartridge driver unit for directing at least one fluid movement within the microfluidic cartridge; b. provide a predetermined condition to the optical unit for performing a quantitative and/or qualitative analysis of the analyte; c. store a data signal obtained from the optical unit; and d. control and monitor an operation of the portable diagnostic apparatus.

In some embodiments, the control unit is configured to provide a predetermined sequence to the cartridge driver unit and a predetermined condition to the optical unit according to the identity of the microfluidic cartridge.

In some embodiments, the portable diagnostic apparatus further comprises a housing for anchoring the cartridge receiving unit, the cartridge driver unit and the optical unit therein.

The cartridge receiving unit further comprises a rail component and a tray. The rail component comprises a pair of slidable rails. The tray is configured to receive the microfluidic cartridge and is anchored on the pair of rails. The rails may slide the tray in and out of the housing such that the microfluidic cartridge may be inserted into the housing.

In some embodiments, the rail component of the cartridge receiving unit, the cartridge driver unit, and the optical unit are mounted within the housing in a configuration such that there is a space for receiving the microfluidic cartridge when the microfluidic cartridge is inserted into the portable diagnostic apparatus, the space comprises one or more microvalve locations, one or more micropump locations and a reaction location corresponding to the position of the one or more microvalves, the one or more micropumps and the reaction site respectively when the microfluidic cartridge is inserted into the space. The heating element of the microvalve controller is mounted proximate to the microvalve location wherein heat can be directed to the microvalve on the microfluidic cartridge when it is inserted. The one or more electrical connector of the micropump controller is mounted juxtapose the one or more micropump locations for electrical connection with the at least one micropump when the microfluidic cartridge is inserted into the portable diagnostic apparatus.

In some embodiments, the optical unit comprises an illumination component comprising a light source and a sensor component comprising a light sensor. The light source and the light sensor are mounted to point towards the diagnostic portion of the microfluidic cartridge.

In some embodiments, the portable diagnostic apparatus further comprises a built-in or removable re-chargeable battery.

In some embodiments, the portable diagnostic apparatus further comprises a switch to trigger the identification unit to read the identity of the microfluidic cartridge when the microfluidic cartridge is positioned in a designated area.

In some embodiments, the portable diagnostic apparatus does not comprise any means for actuation of a fluid outside of the microfluidic cartridge and wherein the portable diagnostic apparatus does not provide any reagents.

In some embodiments, the portable diagnostic apparatus further comprises a user interface unit configured to display the quantitative and/or qualitative analysis of the analyte, wherein the user interface unit is connected to the control unit.

In some embodiments, the cartridge receiving unit and the cartridge driver unit are configured to connect with the microfluidic cartridge when the microfluidic cartridge is fixed at a designated area. The cartridge receiving unit receives and secures the microfluidic cartridge at the designated area. The microvalve controller is juxtapose to at least one microvalve. The micropump controller is electrically connected to at least one micropump. The actuation of fluids and the analyte detection are performed within the designated area during operation.

In some embodiments, the cartridge receiving unit comprises a rail component and a tray. The rail component comprises a cavity for slidably receiving the tray. The tray comprises a cartridge chamber for receiving the microfluidic cartridge such that the microfluidic cartridge is positioned at the designated area.

In some embodiments, the portable diagnostic apparatus further comprises at least one of the following sensors controlled by the controller: a. humidity sensor; b. temperature sensor; such that one or more environmental data may be collected around the time when the microfluidic cartridge is used in the apparatus.

In some embodiments, the portable diagnostic apparatus further comprises a data storage module for storing the one or more of environmental data and diagnostic data; and a transmitter for transmitting the environmental data and the diagnostic data to a remote server.

In some embodiments, the portable diagnostic apparatus further comprises a smart device, wherein the smart device comprises: a. an environmental measuring module for acquiring environmental data, wherein environmental data comprises at least one environmental parameter at the location; b. a data storage module for storing raw data, wherein the raw data comprises one or more of environmental data and diagnostic data; and c. a transmitter for transmitting the raw data to a remote server.

In some embodiments, the environmental data is selected from positioning data, humidity, temperature, and time.

In some embodiments, the smart device can optionally connect to and communicate with the portable diagnostic apparatus and the remote server.

In some embodiments, the smart device further comprises a battery, wherein the battery is rechargeable and can operate 30 days without being recharged.

According to another aspect, provided is a microfluidic cartridge, comprising a microfluidic portion and a diagnostic portion. The microfluidic portion comprises: i.

a plurality of reservoirs capable of holding fluid therein; ii. a plurality of microchannels connecting one or more reservoirs to the diagnostic portion; iii. a plurality of microvalves operable between a close state and an open state for sealing and opening the microchannel connections respectively; and iv. a plurality of micropumps coupled to one or more reservoirs. The microvalves in the closed state allow fluid to be stored and sealed within the reservoirs and the microvalves in the open state allow fluid to flow between the reservoir and the diagnostic portion. The micropump may be actuated to cause fluid movement from the reservoir to the diagnostic portion such that a plurality of reagents can be preloaded and stored in a sealed manner within the microfluidic cartridge until use.

In some embodiments, the diagnostic portion comprises a diagnostic chamber for receiving at least one fluid from the microfluidic portion.

In some embodiments, the microfluidic cartridge further comprises a waste reservoir, the waste reservoir connected to the diagnostic portion via an outlet for receiving waste fluid ejected from the diagnostic chamber.

In some embodiments, the diagnostic portion is at least partially transparent for optical detection.

In some embodiments, the microfluidic cartridge further comprises a microporous membrane configured to remove gas in the sample and/or the reagent.

In some embodiments, the waste reservoir is connected to a microporous membrane to remove gas from the waste.

In some embodiments, at least one reservoir is filled with at least one fluid, wherein the fluid is a reagent and is sealed closed with a microvalve.

In some embodiments, the microfluidic cartridge further comprises a plurality of reagents pre-loaded, sealed and stored separately in a reservoir; and at least one reactant pre-supplied at the diagnostic portion.

In some embodiments, at least one reservoir for holding at least one sample further comprises a sample inlet having a removable cap.

According to another aspect, provided is a portable diagnostic system comprising a portable diagnostic apparatus as described herein and optionally a microfluidic cartridge as described herein.

According to another aspect, provided is a method of detecting at least one analyte from a sample using the portable diagnostic apparatuses as described. The sample is loaded onto a microfluidic cartridge having a diagnostic portion comprising at least one pre-supplied reactant and a microfluidic portion comprising a plurality of microvalves, a plurality of micropumps and a plurality of reservoirs comprising at least one pre-supplied reagent. The microfluidic cartridge is positioned at a diagnosing designated area of the cartridge receiving unit. The method comprises the steps of: a) directing the sample and at least one reagent from the microfluidic portion to the diagnostic portion within the microfluidic cartridge at a predetermined sequence by opening at least one microvalve which seals at least one reservoir of the microfluidic cartridge and actuating at least one micropump in the microfluidic cartridge; b) providing a predetermined condition to the diagnostic portion of the microfluidic cartridge to generate at least one signal; c) detecting the at least one data signal and collecting diagnostic data using an optical sensor; and d) analyzing the diagnostic data to determine the presence of the analyte quantitatively and/or qualitatively.

In some embodiments, further comprising the steps of: a) reading the identity of the microfluidic cartridge; b) providing a predetermined sequence to the cartridge driver unit and a predetermined condition to the optical unit based on the identity of the microfluidic cartridge.

According to another aspect, provided is a method of obtaining disease prevalence information comprising: a. obtaining diagnostic data or sample at a location using a portable diagnostic apparatus of claim 20, wherein the diagnostic data comprises at least one biochemical or pathological measurement of a subject; b. obtaining environmental data of the location; c. transmitting the diagnostic data and the environmental data to a server; d. collecting and storing, in the server, the diagnostic data and the environmental data of a plurality of subjects in a plurality of locations to form a databank; and e. analyzing the databank for disease prevalence information of subjects at the plurality of locations.

According to another aspect, provided is a system for managing a network of portable diagnostic apparatuses, comprising at least one portable diagnostic apparatuses as described herein, at least one user terminal, and a server. The server comprises a data module for collecting and storing raw data, wherein the raw data comprises one or more of the following: (1) diagnostic data obtained at a location using a portable diagnostic apparatus, wherein the diagnostic data comprises at least one biochemical or pathological measurement of a subject, (2) environmental data obtained at the location using an environmental measuring module, wherein the environmental data comprises at least one environmental parameter and (3) apparatus data obtained from the portable diagnostic apparatus; and (4) a data module for analyzing the raw data. The server is connected to the user terminal and to the portable diagnostic apparatus.

In some embodiments, the system further comprises a plurality of portable diagnostic apparatuses, wherein the server is a cloud-based platform connected wirelessly to the user terminal and to the portable diagnostic apparatus.

In some embodiments, the data module performs one or more of the following steps: (1) collects raw data; (2) conducts analysis on the raw data to provide results; and (3) transmits the results to the user terminal. The data module also provides one or more of the following results: (1) disease prevalence at different locations displayed on a map; (2) disease prevalence over a period of time; and (3) severity of a disease in a particular location; and (4) correlation between environmental conditions and apparatus status.

In some embodiments, the data module further comprises one or more access controls to the raw data and the results.

In some embodiments, the server provides technical support or one or more software updates remotely. For example, the server can transmit updated versions of the apparatus software via a network. The server can also provide information regarding how to fix particular machine errors of the apparatus. In some embodiments, the data module can evaluate information about the environmental parameters of the apparatus, such as temperature, humidity, time, and positioning data (e.g., location), and compare it with error codes received by the apparatus to determine whether one or more of the environmental parameters are causing the error codes. The data module can provide solutions in the form of remote technical support to the user, instructions for how to fix the issue, or other forms of technical support. The data module can also send software updates remotely to the smart device.

In some embodiments, the data module further provides correlation between environmental conditions and disease prevalence. In some embodiments, the environmental conditions include temperature, humidity, or time. In some embodiments, the environmental conditions are determine by third party sources and not by the portable diagnostic apparatus.

In some embodiments, the data module further comprises one or more access controls to the raw data and the results.

According to another aspect, provided is a method of using the system as described herein, comprising the following steps: (1) obtaining raw data at the location and storing it on a data storage module; (2) transmitting the raw data from the data storage module to a server; (3) collecting and storing, in the server, a plurality of raw data from a plurality of portable diagnostic apparatuses to form a databank; and (4) analyzing the databank to provide results. The raw data comprises one or more of the following: (1) diagnostic data obtained at a location using a portable diagnostic apparatus, wherein the diagnostic data comprises at least one biochemical or pathological measurement of a subject; (2) environmental data obtained at the location using an environmental measuring module, wherein the environmental data comprises at least one environmental parameter; (3) apparatus data obtained from the portable diagnostic apparatus.

In some embodiments, the raw data is transmitted to the server once an hour, even when the portable diagnostic apparatus is not connected to an external power source.

In some embodiments, the raw data is diagnostic data obtained at a location using a portable diagnostic apparatus, wherein the diagnostic data comprises at least one biochemical or pathological measurement of a subject and location data; and the results provide disease prevalence information.

In some embodiments, the raw data is one or more of temperature, humidity, time, positioning data, and apparatus data; and the results provide information associated with performance of the portable diagnostic apparatus. In some embodiments, the performance of the portable diagnostic apparatus is indicated by the operation status, such as the error codes of the machine, the system voltage, total operation hours, and total number of tests. Results include, but are not limited to, error codes, ways to fix the error codes, and correlation information between the error codes and one or more of temperature, humidity, positioning data, and time.

There are many advantages to various embodiments of the present disclosure such as providing a “one step” solution for detecting analyte on the field. For example, some embodiments provide a diagnostic system with multiple automated features that is capable of providing a one-step solution to near-patient clinical evaluation and diagnosis.

In some embodiments, the diagnostic system is portable and requires minimal intervention by the user. Near-patient testing can be performed by either medical professionals such as physicians and nurses, or by trained laymen such as clinic staff members and caregivers.

The example apparatus for detecting analyte involves relatively small amount or volume of sample (some embodiments from a few microliters (μl) to hundreds of μl) while using an integrated reaction-to-detection instrument/methodology. As such, this is a genuine “field testing equipment” that will provide true convenience to field personnel. As a result, special handling and transportation of analyte to the laboratory and the excessive transportation time that may affect the quality of analyte are greatly reduced.

Another advantage is that the apparatus of some embodiments requires little or no sample preparation compared to conventional diagnostic method or system, thereby reducing processing time.

Another advantage of some embodiments is that it can be applied in various area of diagnosis and food safety analysis. For example, a method of detecting one or more analyte(s) associated with the presence of a disease in a subject. The application includes, but is not limited to animal immunodiagnostics (e.g. Swine Influenza virus (e.g. H1N1) infection, Porcine Reproductive and Respiratory Syndrome (PRRS), Bovine Foot-and-Mouth Disease (FMD), Classical Swine Fever (CSFV) infection, and Bovine Spongiform Encephalopathy (BSE) Infectious Disease), food safety test (e.g. detection of food allergens (e.g. peanuts, seafood), aflatoxin and melamine), the clinical detection for human subjects (e.g. the detection of infectious diseases (e.g. sexually transmitted diseases (STD), Middle East respiratory syndrome coronavirus (MERS-CoV) and Influenza virus infection), tropical diseases (e.g. Dengue virus and Japanese Encephalitis virus infection) and new emergent infectious diseases which fall within antigen/antibody immunological mechanism in their pathological pathway), Flu A, flu B, RSV, HPIV, adenovirus, dengue, chikungunya, Zika, malaria, leptospirosis, toxoplasmosis, canine distemper virus Ab, canine parvovirus Ab, or heartworm. Some of the implementations can be adapted to analyze for multiple analytes within the same sample and same process, significantly reducing the cost and processing time involved for the checking for multiple diseases/analytes.

Example embodiments are configured specifically to enable performance of all required steps on a single tray without user intervention. The steps include (1) reactions in the microfluidic cartridge (2) detection of the signal from the microfluidic cartridge and (3) analysis and display of the results to the user. They provide an automated rapid diagnostic apparatus which minimize human interaction and chances of human error. It provides a one-step, fool-proof solution to near-patient rapid diagnostic that can be used by a layman with minimal training.

In some example embodiments, e.g. the microvalve controller, the actuation component provides sealed compartments for storing at least one reagent within the reservoirs and active, precise actuation of at least one fluid within the microfluidic cartridge.

The reaction and detection of some example embodiments, e.g. the single tray system, may take place at the same cartridge without the need for separation of the diagnostic portion from the detection portion. The microfluidic cartridge e.g. for the pre-loaded chip embodiment, is self-contained, i.e., pre-supplied (or pre-loaded) with all required reagents within the microfluidic cartridge for reaction during manufacturing process such that no reagents are required on site.

As a summary, various embodiments provide various advantages such as low cost, time-and-space saving, portable, requiring minimal resources and low degree of skills and technicians to conduct a complete analyte detection rapidly at scale and on site efficiently.

### BRIEF DESCRIPTION OF THE DRAWINGS

FIG. 1 is a block diagram illustrating a diagnostic system, according to one embodiment of the present invention.

FIG. 2 is a schematic view of the diagnostic system, according to an example embodiment of the present invention.

FIG. 3 is a schematic bottom view of a tray of the diagnostic system with (RIGHT) and without (LEFT) the diagnostic chip, according to an example embodiment of the present invention.

FIG. 4 is a schematic view of the fluid control component of the microfluidic cartridge operating unit of the apparatus, according to an example embodiment of the present invention.

FIG. 5 is a schematic view of an optical inspection unit of the diagnostic system, according to an example embodiment of the present invention.

FIG. 6A is a schematic top view (left), side view (middle) and bottom view (right) of a microfluidic cartridge, according to an example embodiment of the present invention.

FIG. 6B is a schematic exploded view of a microfluidic cartridge, according to an example embodiment of the present invention; FIG. 6C is a schematic exploded view of a microfluidic cartridge, according to another example embodiment of the present invention.

FIG. 7 is a flow chart of the production sequence for the microfluidic cartridge, according to another example embodiment of the present invention.

FIG. 8 is a flow chart of the coating process and the deposition of detection spots on the diagnostic chip, according to one embodiment of the present invention according to another example embodiment of the present invention.

FIG. 9 depicts the detection of fluorescent antigen (FluA), according to an example embodiment of the present invention.

FIG. 10A is a block diagram of a portable diagnostic apparatus, according to an example embodiment of the present invention.

FIG. 10B is a block diagram of a portable diagnostic apparatus, according to another example embodiment of the present invention.

FIG. 11A is a schematic top view (left), side view (middle) and bottom view (right) of a microfluidic cartridge, according to an example embodiment of the present invention.

FIG. 11B is a schematic exploded view of a microvalve membrane and a top part of a microfluidic cartridge, according to the same example embodiment of the present invention as illustrated in FIG. 11A.

FIG. 11C is a schematic exploded view of a microfluidic cartridge, according to the same example embodiment of the present invention as illustrated in FIG. 11A.

FIG. 12A is a schematic exploded view of a microfluidic cartridge, according to another example embodiment of the present invention.

FIG. 12B is a schematic top view of the microvalve membrane of a microfluidic cartridge, according to the same example embodiments of the present invention as illustrated in FIG. 12A.

FIG. 12C is a schematic top view of the top part of a microfluidic cartridge, according to the same example embodiments of the present invention as illustrated in FIG. 12A.

FIG. 12D is a schematic bottom view of the top part of a microfluidic cartridge, according to the same example embodiments of the present invention as illustrated in FIG. 12A.

FIG. 12E is a schematic top view of the bottom part of a microfluidic cartridge, according to the same example embodiments of the present invention as illustrated in FIG. 12A.

FIG. 12F is a schematic bottom view of the bottom part of a microfluidic cartridge, according to the same example embodiments of the present invention as illustrated in FIG. 12A.

FIG. 13A and 13B are schematic views of fluid movement within a microfluidic cartridge driven by a cartridge driver unit according to an example embodiment.

FIG. 14A is a schematic exploded view of a portable diagnostic apparatus, according to an example embodiment of the present invention.

FIG. 14B is a schematic exploded view of another example of a portable diagnostic apparatus.

FIG. 15A is a top perspective view of a microvalve controller of a microfluidic cartridge driver unit, according to an example embodiment of the present invention.

FIG. 15B is a bottom perspective view of the same microvalve controller as illustrated in FIG. 15A.

FIG. 16A and FIG. 16B are schematic views of a microfluidic cartridge receiving unit, according to an example embodiment of the present invention.

FIG. 16C is a schematic view of a tray of the same microfluidic cartridge receiving unit as illustrated in FIG. 16A.

FIG. 16D is a schematic view of the same tray as illustrated in FIG. 16C but with a microfluidic cartridge inserted therein.

FIG. 17 is an exploded view of an optical unit and an identification unit of the diagnostic apparatus, according to an embodiment of the present invention.

FIG. 18 is a schematic view of an assembly of an optical unit, an identification unit, a cartridge driving unit and a cartridge receiving unit of the diagnostic apparatus, according to an example embodiment of the present invention.

FIG. 19 is a flow chart of an operation of a diagnostic apparatus, according to an example embodiment of the present invention.

FIG. 20 is a schematic view of a system for managing a network of field diagnostic apparatuses and obtaining disease prevalence or other information, according to an embodiment of the present invention.

FIG. 21 is a flow chart of a method of obtaining disease prevalence or environmental information, according to an embodiment of the present invention.

FIG. 22 is a flow chart of an operation of the system of FIG. 20, according to an embodiment of the present invention.

FIG. 23 is a flow chart illustrating the flow of information and data throughout the various components of a system according to an embodiment of the present invention.

FIG. 24 is a schematic view of an assembly of smart device and units with which it interacts, according to an embodiment of the present invention.

### DETAILED DESCRIPTION

As used herein and in the claims, “comprising” means including the following elements but not excluding others.

As used herein and in the claims, “couple” or “connect” refers to connection either directly or indirectly via one or more physical means unless otherwise stated.

As used herein and in the claims, “microfluidic” refers to precise control and manipulation of fluids or liquids that are geometrically constrained to a small, sub-millimeter scale at which capillary penetration governs mass transport below 0.01ml. Microfluidic systems disclosed herein does not include paper-based microfluidic systems.

“Microfluidic cartridge” refers to a cartridge with microfluidic structures comprises a variety of components, modules, chambers, etc. that are fluidly connected and configured to process a fluid sample. Microfluidic cartridges disclosed herein performs biological or biochemical assays such as immunoassays. Microfluidic cartridges disclosed herein does not include applications for polymerase chain reaction (PCR) or nucleic acid sequencing.

“Immunoassay” refers to tests involving coupling of an antibody or an antigen to a molecule for the detection of an analyte. The molecule can be a molecule that can generate a fluorescent signal or other detection signals.

“Sample” refers to a substance to be tested and includes, but is not limited to, a blood sample, a blood serum sample, a urine sample, a sweat sample, a saliva sample, a tear drop sample, a nasal swap, a nasopharyngeal swab, or a sample comprising other bodily fluids or other non-human samples. In some embodiments, a sample is processed such that in can be tested by a microfluidics system. For example, a solid sample may be treated with buffers or other reagents in order to isolate or extract the analyte of interest.

As used herein and in the claims, “analyte” refers to, but not limited to, pathogens and biomolecules present in e.g. body fluids, nasal swaps or blood serum sample from a target individual, including, but not limited to, e.g. animal or human subjects. It shall be understood that when the term “analyte” is used, it may refer to one or more analytes.

As used herein and in the claims, “reactant” refers to, but not limited to, target substance that reacts with one or more analyte(s), such as an antibody or an antigen. The substance may be immobilized on a diagnostic chip for use. It shall be understood that when the term “reactant” is used, it may refer to one or more reactants.

As used herein and in the claims, “fluid” refers to any liquids including, but not limited to, liquid samples, reagents, buffers.

As used herein and in the claims, “diagnostic” refers to detecting analyte not only limited to disease-related analyte(s). However, the diagnostic system described herein does not include amplification of nucleic acids such as polymerase chain reaction (PCR) or nucleic acid sequencing.

As used herein and in the claims, “pre-supplied” or “pre-loaded” refers to the fluids such as reagents and/or reactants are supplied or loaded during the manufacturing process of the microfluidic cartridge such that users do not need to supply or load the fluids.

As used herein and in the claims, “micropump” refers to a fluid actuator that actuates at least one fluid.

As used herein and in the claims, “microvalve” refers to a barrier between the channels and/or reservoirs. In some example embodiments, the microvalve is a closed valve under resting position and can be opened under active operations such that fluids or reagents can be pre-sealed within the reservoirs for long-term storage.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

It will be further understood that when an element is referred to as being “top” or “bottom”, these words are used to describe the relative position between the elements. Thus, a “top” part, element, component, region, layer or section discussed below could be termed a “bottom” part, element, component, region, layer or section without departing from the teachings of the present application.

### EXAMPLE 1

### 1. Apparatus

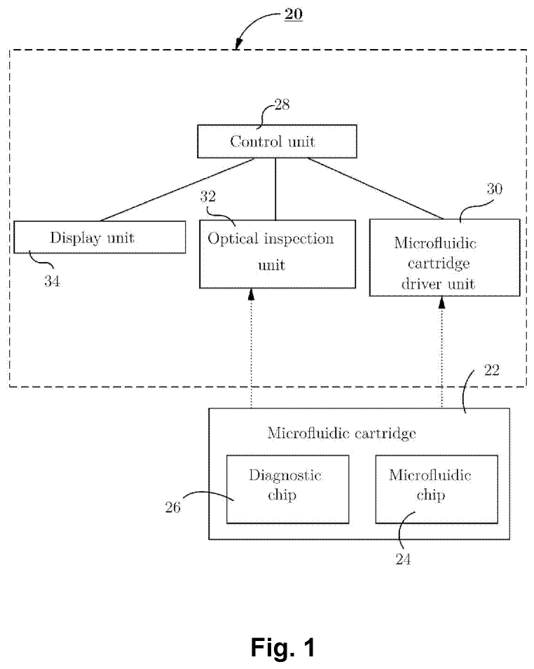

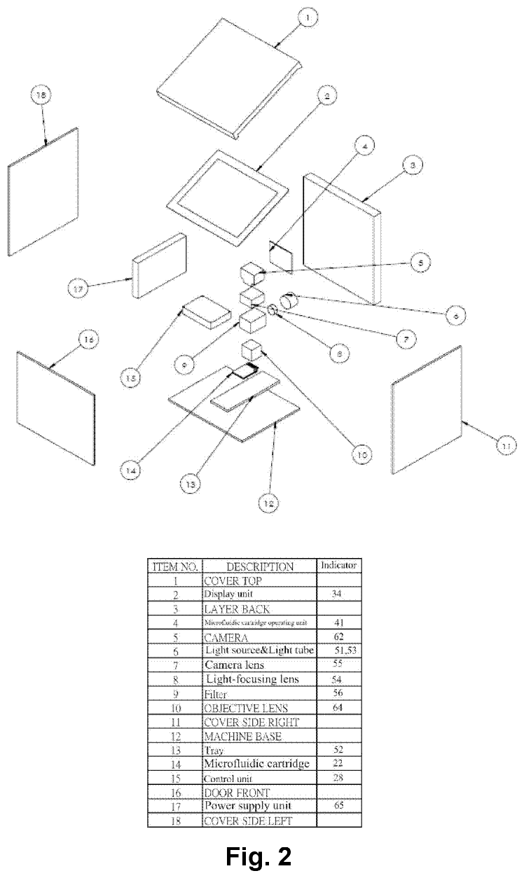

FIG. 1 and FIG. 2 show a diagnostic system comprising a (1) diagnostic apparatus 20 and (2) a microfluidic cartridge 22, which operates with the diagnostic apparatus 20. The microfluidic cartridge 22 which includes a microfluidic chip 24 and a diagnostic chip 26 is configured to collect and manipulate at least one sample, which may include at least one analyte. The microfluidic cartridge 22 also receives and/or holds at least one reagent. The diagnostic apparatus 20, which is a portable, hand carriable and compact device, includes a control unit 28, a microfluidic cartridge driver unit 30, an optical inspection unit 32 and a display unit 34. The control unit 28 controls and is connected to the microfluidic cartridge driver unit 30, the optical inspection unit 32 and the display unit 34. The microfluidic cartridge driver unit 30 is configured to receive and drive the microfluidic cartridge 22 such that the sample collected and the reagent run through the microfluidic chip 24 and the diagnostic chip 26 in a predetermined sequence. After the reaction is done in the predetermined sequence, the microfluidic cartridge driver unit 30 also allows inspection or analysis of the diagnostic chip to occur on the same microfluidic cartridge in the same location or position. The optical inspection unit 32 is configured to inspect the diagnostic chip 26 on the same microfluidic cartridge in the same location where the reaction is done for analyzing the presence of analyte. The display unit 34 is configured to display relevant information including analyzed/diagnostic results to users. FIG. 2 shows that the control unit 28, microfluidic cartridge driver unit 30, optical inspection unit 32, display unit 34 and power supply unit 65 are enclosed in a self-contained diagnostic apparatus. In one example embodiment, the apparatus includes a microfluidic cartridge receiving hole on its front panel for receiving the microfluidic cartridge 22.

### 1.1 Microfluidic Cartridge Driver Unit

1.1.1 Tray and Cartridge chamber

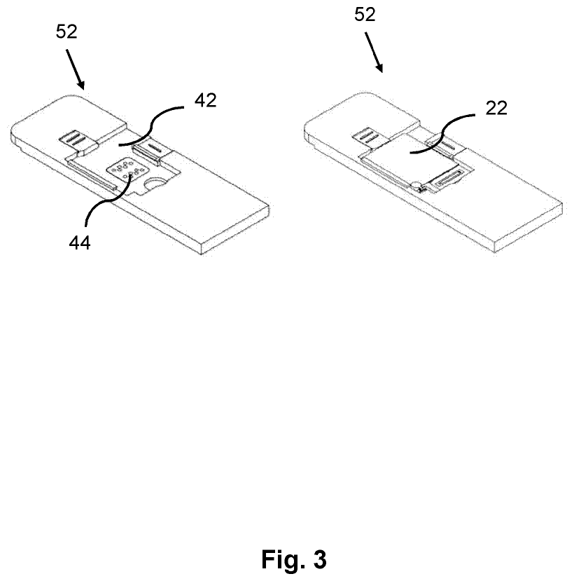

The microfluidic cartridge driver unit comprises a tray 52 and a cartridge chamber 42 (see FIG. 3).

The cartridge chamber 42 is configured for receiving the microfluidic cartridge 22. The tray 52 comprises the cartridge chamber 42, which receives the microfluidic cartridge 22. Tray 52 serves as the same location or position for (1) the reaction to be run in a predetermined sequence and (2) inspection and analysis of the diagnostic chip. In one further example embodiment, the electrical connectors 44 is situated underneath the microfluidic cartridge 22 and acts as an interface for the microfluidic cartridge 22 to drive/control and provide power/electrical current to the microfluidic cartridge 22.

The one-tray system eliminates the possibility of human error by design. FIG. 3 shows the empty tray (left) and the tray with the microfluidic cartridge 22 fitted in (right).

The microfluidic cartridge 22 and the cartridge chamber 42 are configured such that there is only one possible way that the microfluidic cartridge 22 can fit into the cartridge chamber 42 and the tray 52 and thus eliminates chances of putting the diagnostic chip 26 of the microfluidic cartridge 22 in undesired orientation or position.

In one example embodiment, the tray has no anchoring system and has a tolerance of >0.5 mm. The microarray is physically small, and the camera captures a small area (approximately 2×3 mm²).

In one example embodiment, an anchoring system is added to the tray 52 to ensure that the diagnostic chip is secured in the tray 52. In a further example embodiment, the anchoring system consists of two anchoring clips, positioned orthogonal to each other. The two anchoring clips working together greatly limits the movement of the diagnostic chip once it is placed into the tray. Less movement means less variation in the possible position of the bioassay and hence increase in detection accuracy and precision. The addition of extra anchoring clips lowers the tolerance to 0.1 mm and allows more accurate detection as the variation in bioassay position is minimized.

In one example embodiment, the microfluidic cartridge driver unit 30 includes a rail system comprising at least one tray rail which guide the tray and ensure that the bioassay is placed directly underneath the light source 51 and the camera 62. The rail system configured to receive at least two edges of the tray and enable expansion in length directions while maintaining the tray with the microfluidic cartridge 22 above a mounting structure to which the rail system is configured to secure.

### 1.1.2 Microfluidic Cartridge Operating Unit

The microfluidic cartridge driver unit 30 comprises a microfluidic cartridge operating unit 41 which controls the fluid movement of the microfluidic cartridge 22 (see FIG. 2). The microfluidic cartridge operating unit 41 includes electrical connectors 44 that act as an interface for the microfluidic cartridge 22 to drive/control and provide power/electrical current to the microfluidic cartridge 22 to perform predetermined sequences for driving the reagent and sample from the microfluidic chip 24 to the diagnostic chip 26 for reaction.

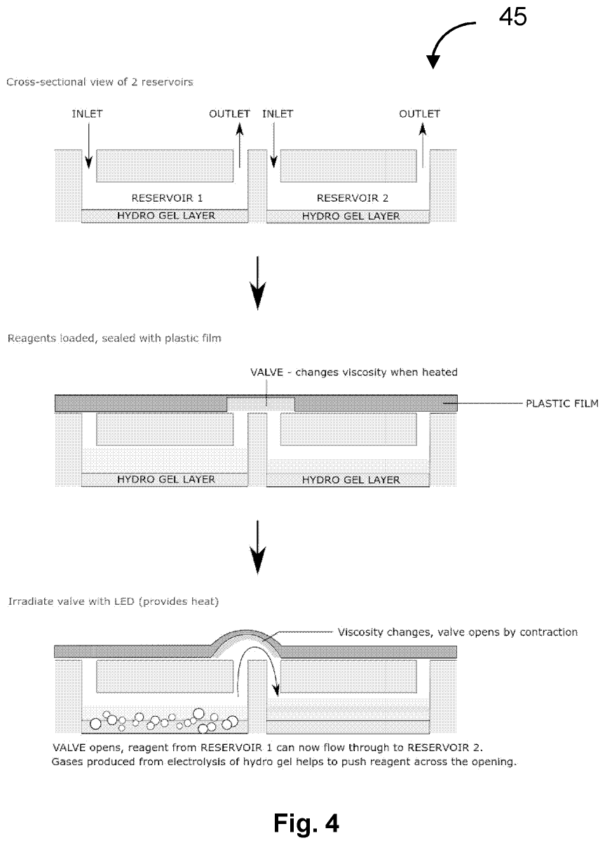

FIG. 4 shows the fluid control component 45 on a microfluidic cartridge operating unit 41. More specifically, the figure shows a cross sectional view of two reservoirs on a microfluidic cartridge operating unit. In some example embodiments, the microfluidic cartridge operating unit 41 further comprises at least one fluid control component 45 configured to control at least one fluid movement within the microfluidic cartridge. The fluid control component 45 is located in close proximity to the cartridge chamber. In one example embodiment, the fluid control component 45 is located less than 10cm away from the cartridge chamber. The fluid control component 45 changes the viscosity of at least one specific part of the microfluidic cartridge by controlling irradiating light which provides the heat on specific parts of the microfluidic chip and opening a valve which facilitate the fluid movements. Gases produced from the electrolysis of hydro gel assist pushing the reagent across the opening. In one example embodiment, the specific part is a plastic film that seals the microfluidic cartridge. The predetermined sequences are discussed in detail herein and are also shown in FIG. 4. Irradiating the plastic film covering the different reservoirs in a particular order cause the reagents to leave the reservoirs and start the reactions in a particular order e.g. washing buffer first, then blocking buffer, then antibodies, then washing buffer. Control signals and power are provided to the fluid control component 45.

### 1.2 Optical Inspection Unit

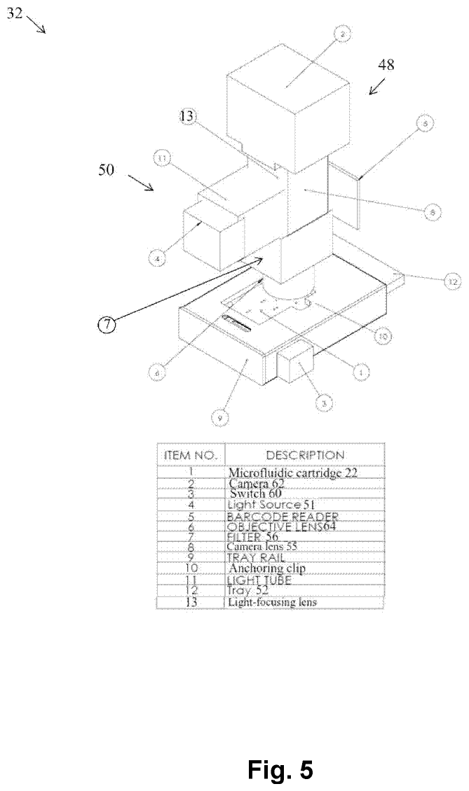

The optical inspection unit 32 as shown in FIG. 5 weighs less than a few kilograms (kg) and can be used for on-site analyte analysis/detection. The optical inspection unit 32 includes an optical sensor 48 and illumination system 50. In some embodiments, the optical inspection unit 32 further includes one or more filters 56. In some embodiments, the optical inspection unit 32 further includes a reader 58 and a switch 60. The tray of microfluidic cartridge driver unit 30 is below the optical inspection unit 32. The microfluidic cartridge driver unit 30 is configured such that when the microfluidic cartridge is fit or secured in the microfluidic cartridge driver unit 30, the diagnostic chip of the microfluidic cartridge is directly below the optical inspection unit 32 for inspection. The illumination system 50 includes at least one light source 51. In some embodiments, the illumination system 50 includes at least one light source 51 and/or at least one light-focusing lens 54. In one example embodiment, the light source 51 can be a laser or LED, either monochromatic or polychromatic. This light source 51 should be strong enough to excite fluorophores. In one example embodiment, the light source 51 can be a high luminosity LED spot light with a blue LED color, for example, a high luminosity LED spot light HBF-00-08-1-B-5V from TMS lite. The specification of TMS lite high luminosity LED spot light HBF-00-08-1-B-5V is shown in the following Table 1 and Table 2.

TABLE 1

| Mechanical information | |
|---|---|
| Casing Material | Aluminium |
| Storage Temperature | Temp 0-45° C., |
| Range | Humidity 20-85% |
| Weight | — |
| Outer Diameter | 30 |
| Inner Diameter | 6 |
| Thinkness | 69 |

In another example embodiment, the illumination system 50 comprises a diode laser radiating at least one laser beam with at least one predetermined wavelength on the diagnostic chip 26 to generate at least one signal. The predetermined wavelength of the laser beam is selected such that at least one signal which is detectable by the optical sensor 48 can be generated. The intensity and the wavelength of the laser beam can be selected/controlled by the user through the control unit 28 for detecting a particular analyte. The laser beam is steered to the diagnostic chip 26 at an angle so as to avoid reflections and to generate the signal at higher quality. The predetermined wavelength, for example, is in a range of 465 to 500 nm, 400 to 700 nm, 430 to 465 nm, 500 to 550 nm, 550 to 580 nm, 580 to 620 nm, or 620 to 700 nm.

In one example embodiment, the light source 51 comprises a light tube 53 that evenly release light. The light tube 53 is configured such that it directs the light to the light-focusing lens 54 or other optical components and helps to focus the beam of light onto the bioassay on the diagnostic chip. The light tube 53 is aligned with the bioassay on the diagnostic chip which is located at a specific position on the microfluidic cartridge and the position of the microfluidic cartridge is determined by the tray and the tray rail.

In one example embodiment, the illumination system 50 comprises at least one light-focusing lens 54 such that the focusing of the light from the light source 51 is optimized. In some embodiments, the light-focusing lens 54 is located on the end of the light tube 53 opposite of the light source 51. In some embodiments the light-focusing lens 54 is located in front of the light source 51. Both the light-focusing lens 54 and the camera 62 can each be separately mounted or secured onto one or more frames to prevent any undesired movements during the optical setup. In one example embodiment, the light-focusing lens 54 can be a convex lens with a focal length of 25.4 mm, for example, LB1761-N-BK7 Bi-Convex Lens, Ø1 in, f=25.4 mm, uncoated from Thorlabs Inc. The specification of the light-focusing lens 54 is shown as below:

Design wavelength: 587.6 nm

Focal length: f=25.3±1%

Back focal length (REF): bf=22.2 mm

Clear aperture: >90%

Surface quality: 40-20 scratch-Dig

Centration: <3 arc min

Diameter tolerance: +0.0/−0.1 mm

Thickness tolerance: ±0.1 mm

The microfluidic chamber is located beneath the optical sensor 48 and the illumination system 50 when the tray 52 at its docked position. The optical sensor 48 includes a camera 62 and at least one objective lens 64. In some embodiments, the optical sensor 48 further includes at least one camera lens 55. The optical sensor 48 receives signals from the diagnostic chip 26 generated by radiating a laser light on the diagnostic chip 26 of the microfluidic cartridge 22 held on the tray of the cartridge chamber by the illumination system 50. The received signals are then sent to the control unit 28 for analysis. The optical sensor 48 can be of a high quantum efficiency in the wavelength range that it is detecting in. In one example embodiment, the camera 62 of the optical sensor 48 can be a charge-coupled device (CCD) or any other suitable camera. In one example embodiment, the camera 62 is a near-infrared optimized camera with a type 2/3 (11.0 mm diagonal) CCD sensor.

The camera lens 55 of the optical sensor 48 can be any suitable lens for camera or a lens of a higher quality such as microscope-grade lens, depending on the type of immunoassay used. In one example embodiment, the camera lens 55 is responsible for assisting the camera to focus on the bioassay since the bioassay is physically small. In one example embodiment, the camera lens 55 is a C-mount lens. In some embodiments, the C-mount lens is located between the camera and the objective lens. In some example embodiments, the C-mount lens is securely attached to the camera. In one example embodiment, the C-mount lens has a focal length of 16 mm. In one example embodiment, the objective lens 64 of the optical sensor 48 is a plan achromat, 4× magnification, 0.1 numerical aperture with a working distance of 18.5 mm, for example, RMS4×-4× Olympus Plan Achromat Objective, 0.10 NA, 18.5 mm WD from Thorlabs, Inc.

In one example embodiment, the optical inspection unit 32 further includes one or multiple filter(s) 56. The filter(s) 56 can be used to filter out any light produced from the light source that is of unwanted wavelength and any undesirable noise in the signals that the camera picks up. One or more than one filters 56 can be used depending on the light source and the fluorophore used. The illumination system 50 is connected to the filter. In one example embodiment, the filter(s) 56 is mounted and aligned between a camera lens 55 and the objective lens 64. This allows any light of unwanted wavelengths to be filter out and only light of a specific wavelength or range of wavelengths to pass through and reach the bioassay. The camera 62 is connected to the camera lens 55 and these two components are connected to the filter(s) 56. This connection to the filter(s) 56 allows any undesirable signal such as noise, that is often of a different wavelength, either produced by the bioassay or by any undesirable reaction, to be filtered and hence minimize unwanted interference with real signals. The filter(s) 56 is connected to a light tube which helps to focus the filtered light beam onto the bioassay. In one example embodiment, a fluorescence filter set for FITC Fluorescein with emission wavelength 513-556 nm and excitation wavelength 467-498 nm is used, for example, 67-004 fluorescence filter set for FITC Fluorescein from Techspec. The specification of 67-004 fluorescence filter set for FITC Fluorescein is shown as follows:

Compatible Fluorophore: FITC

Coating: Hard Coated

Dichroic Cut-On Wavelength (nm): 506.00

Dichroic Filter: #67-080

Emission Filter: #67-031

Emission Wavelength (nm): 513-556

Excitation Filter: #67-028

Excitation Wavelength (nm): 467-498

Manufacturer: EO

Substrate: Fused Silica

Type: Fluorescence Filter Kit

Wavelength Range (nm): 467-556

RoHS: Compliant

In some example embodiments, the optical inspection unit 32 further includes a switch 60.

In a further example embodiment, the switch 60 is a microswitch. The microswitch is attached to the back of the tray rail and is electrically connected to the power supply unit 65. The microswitch is automatically activated when the tray 52 is pushed into the docked position through the tray rail. Upon activation, the microswitch can switch on a reader to read the identity of the microfluidic cartridge.

In some example embodiments, the optical inspection unit 32 further includes a reader 58 to read the the identity of the microfluidic cartridge. In one example embodiment, the reader 58 is a barcode reader. The barcode reader can read the 2D barcode attached or fixed on the microfluidic cartridge.

In one example embodiment, the user manually select a suitable program to run the diagnostic chip or microfluidic cartridge. In some embodiments, the program uses a particular pre-determined electric pulse sequences to drive the appropriate reactions, analyse, and calculate the results using the appropriate size of the microarray.

In some example embodiments, an internal barcode reader is incorporated into the system, where the barcode reader is located above where the microfluidic cartridge will be placed.

In one example embodiment, a barcode is attached or fixed onto the microfluidic cartridge (FIG. 5). In a further example embodiment, a two-dimension (2D) code is attached or fixed onto the microfluidic cartridge. A 2D code that incorporates the identity of the microfluidic cartridge, the analytes or diseases to be tested, expiry date of the chip is placed on the microfluidic cartridge during manufacturing process. Upon the insertion of the microfluidic cartridge into the tray, the barcode reader is activated and a 2D code is scanned automatically by the barcode reader. The software can automatically choose the correct program to use based on the 2D code. This feature eliminates the need to manually select the pulse program thus makes the design more user-friendly and less prone to human error In one example embodiment, the software can also identify a microfluidic cartridge which has been previously used or which is defective. The software shows a warning message on the screen and will not proceed with the program.

The components in the optical inspection unit 32 are arranged such that a compact integrated optical inspection unit is formed. This compact design leads to a smaller, and lighter diagnostic system. The diagnostic system should be small and light enough to be moved from clinic to clinic if needed. In one example embodiment, the present invention is small and light enough to be hand-carried onto a domestic aircraft. In one example embodiment, the dimension of the apparatus is approximately 30×30×30 cm³ and the weight is approximately 5-6 kg.
The events associated with the optical inspection unit 32 are described below:
   S1. The user puts the microfluidic cartridge into the tray or the microfluidic chamber.
   S2. The user pushes the tray into the apparatus as guided by the tray rail.
   S3. Switch is activated as the tray is pushed in.
   S4. The switch switches on the barcode reader.
   S5. The reader reads a code that is printed onto the microfluidic cartridge.
   S6. The code, which contains the identity of the microfluidic cartridge, it prompts the software of the control unit to automatically selects the program that is associated with this microfluidic cartridge.
   S7. Once the software has selected the correct program, a specific sequence of electrical pulses is generated and the electrical pulse sequence passes through the microfluidic cartridge via the electrical connectors that are located at the bottom of the tray. This sequence of electrical pulses will drive reagents in the microfluidic chip out of their reservoirs and push them into the reaction chamber in a pre-determined sequence. The electrical pulses can also drive the fluid control component 45 to facilitate the fluid movement within the microfluidic cartridge.
   S8. Once the reaction is completed in the reaction chamber, the illumination system is activated and the bioassay is excited by a light beam.
   S9. The optical sensor captures an optical image and the image is analyzed by the software.
   S10. The result is shown on the screen for the user to see. No human interpretation is required.

### 1.3 Control Unit

The control unit 28 generally includes a microprocessor (CPU), memory, and input/output (I/O) interfaces. The control unit 28 controls the quantitative and qualitative analysis, interfacing, and storage of signal obtained from the optical inspection unit 32, and controls and monitors all the operations of the diagnostic apparatus 20.

The control unit 28 further includes a non-transitory computer readable medium to store computer readable codes such that when the code is executed by the microprocessor, it instructs all the parts of the diagnostic apparatus 20 to perform and operate the steps as described above and herein. The non-transitory computer readable medium may comprise any known type of data storage and/or transmission media, including magnetic media, optical media, random access memory (RAM), read-only memory (ROM), a data cache, a data object, etc. Moreover, memory may reside at a single physical location, comprising one or more types of data storage, or be distributed across a plurality of physical systems in various forms.

In one embodiment, the control unit 28 comprises of software modules which might be needed for system operation. The modules include an operating system, an application module, an image processing module, a microfluidic cartridge driver software module for controlling the flow of fluids in the microfluidic chip 24 as aforesaid and a user interface software module. The operating system manages computer hardware resources and provides common services for all the computer software modules. The operating system can be Apple iOS, Android, Microsoft windows or Linux. The operating system is also integrated various communication protocols, be it wired or wireless, such local area network (LAN), USB, Wi-fi, Bluetooth, etc. The application module is a set of programs designed to carry out operations for the apparatus. It manages the data of the apparatus as well as job data, program data, client data, microfluidic cartridge data, pump setting, optical sensor setting, and the data collected from the optical inspection unit 32. The image processing module collects the data from the optical inspection unit 32. The image processing module selects areas of interest of the diagnostic chip 26 and controls the acquiring of images therefrom. The image processing module also corrects the brightness and contrast of the images acquired. Upon receiving these images from the image processing module, the control unit 28 measures and compares the images of the diagnostic chip 26 according to the setting of the optical sensor 48. The image processing module then counts and calculates according to the set limits and sends the analyzed results to the user interface software module. The microfluidic cartridge driver software module is designed to instruct the microfluidic cartridge driver unit 30 to control the electrical current and the time of delivering such electrical current to the microfluidic pump at the microfluidic chip 24. The higher the electrical current and/or the longer the time for delivering such electrical current, the more fluids can then be pumped from the reservoirs 80. The user interface software module is the interface that allows users to interact with the apparatus through graphical icons, visual indicators such as notations and commands. The user interface software module makes the apparatus very user-friendly to non-skilled persons by allowing the user to obtain, understand, add, edit and delete information easily without any special skills. It also allows user to feel that they have close connections with the optical inspection unit 32, with the help of interactivity of graphic, sound, as well as the delivery of notifications and commands given by the user interface software module.

### 1.4 Power Supply Unit

A power supply unit 65 is provided in the apparatus. The power supply unit 65 includes at least one rechargeable battery pack, battery charger port, power switch, and power management electronic circuit. The conventional rechargeable battery pack can be made of lithium ion, lithium polymer or other high capacity battery. The rechargeable battery pack in the power supply unit 65 can support a few hours of operation of the apparatus without public electrical supply, say in remote locations. The power supply unit 65 is equipped with a battery protection circuitry which can protect the rechargeable battery pack against over charge, over current and over temperature so as to guarantee the safety of the apparatus and user. The power supply unit 65 is also equipped with a battery connector to let the user replaces the fully discharged battery by a spare fully charged battery when there is an extended hours of use. The power management electronic circuit is used for converting the rechargeable battery pack voltage to different voltage as required by different system units. The power management electronic circuit is connected to the control unit 28, the rechargeable battery pack, the microfluidic cartridge driver unit 30 and the optical inspection module. The power management electronic circuit allows the initiation, termination and alteration of the voltage whenever it is needed to save the power consumption of the apparatus. These command signals are given by the control unit 28. Moreover, the battery charger provides Direct current (DC) to charge up the rechargeable battery pack in the system via the battery charger port at the back panel of the apparatus. The apparatus can operate even when the rechargeable battery pack is empty but when public or external electric supply is presented. The battery charger port can be detached when the rechargeable battery pack is charged. In one example embodiment, the diagnostic platform can operate with either plug-in power supply or solely on battery. The use of a rechargeable battery allows the apparatus to be taken outdoor and be used in rural areas where electricity supply may be scarce.

In one example embodiment, the specification of the battery is shown as below:

Type: RRC2024

Voltage: 14.40V

Capacity: 6.60 Ah

Max. charge current: 4.62 A

Max. charge voltage: 16.80V

Max. discharge current: 10.00 A

Dimensions: (L×W×H) 167.7 mm×107.6 mm×21.8 mm (max.)

Weight: 590 g

In one example embodiment, the battery can be hand-carried onto a domestic flight and can be shipped internationally when installed on the diagnostic platform.

In one example embodiment, the fully-charged battery supports at least around 5 hours of operation of the apparatus.

In one example embodiment, the battery is rechargeable and it is replaceable by users. The battery allows the apparatus to operate in areas without electricity or temperature control.

### EXAMPLE 2

### 2. Microfludic Cartridge

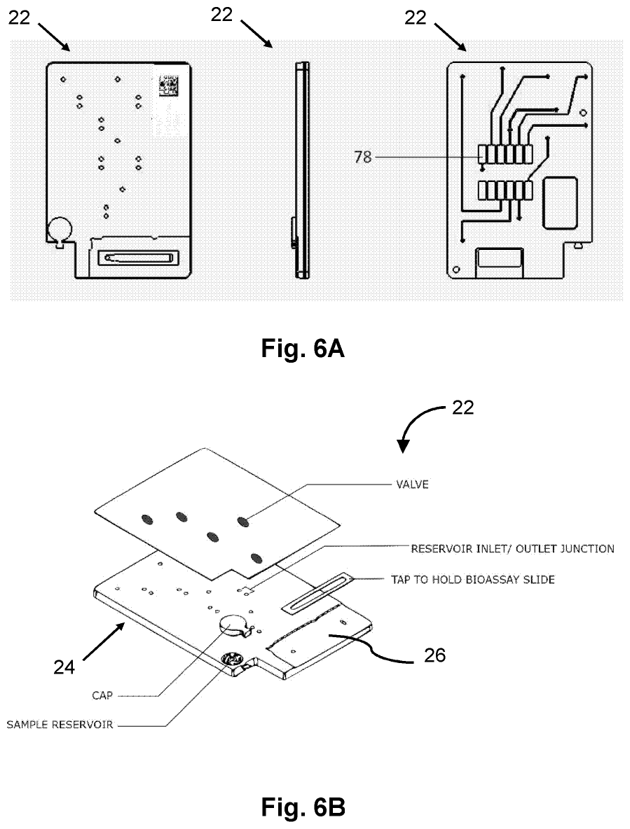

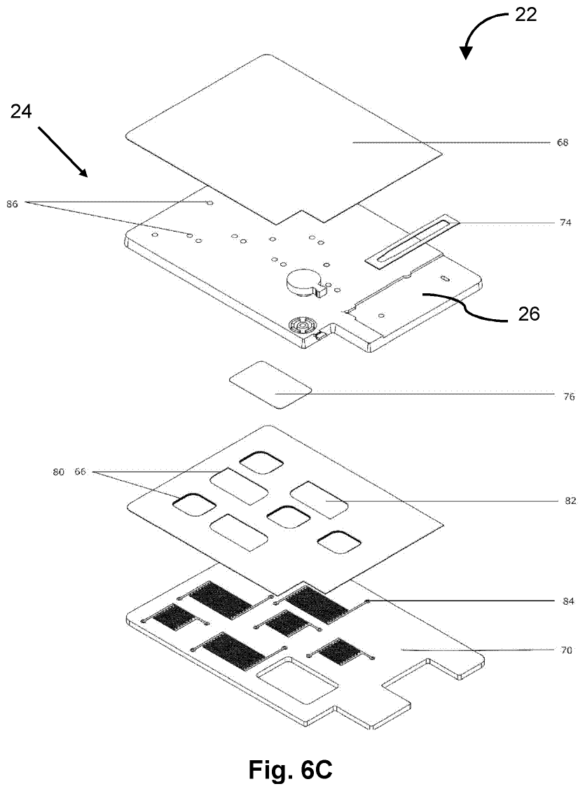

The microfluidic cartridge 22 as shown in FIG. 6A, FIG. 6B and FIG. 6C includes the diagnostic chip 26 fixed to the microfluidic chip 24. In the implementation shown, it has a dimension smaller than a credit card with a thickness of 1-10 mm. The microfluidic chip 24 includes an electrical connecting interface 78 for receiving control signals and power provided through electrical connectors 44 of the cartridge chamber 42, a top part 68 and a bottom part 70 attached to the top part 68. In this example, the top part 68 and bottom part 70 are assembled together with adhesive materials or by welding process. The bottom part 70 may be made of electrical insulated material such as plastic and resin material. As shown in FIG. 6A, the top part 68 has a plurality of micro grooves 66, a channel opening having a fluid connection with the microfluidic chip 24 and an adhesive 74 for attaching the microfluidic chip 24 at the channel opening. The bottom part 70 of the microfluidic cartridge has a groove for a microporous membrane 76 to be placed therein as shown in FIG. 6C. In one example embodiment, the top part 68 includes a plastic film that seals the microfluidic cartridge. The plastic film receives light control signals provided by the fluid control component 45 of the microfluidic cartridge driver unit. When sufficient light control signals are received at a particular valve location on the microfluidic chip 24, the plastic film covering said valve changes viscosity. The change in viscosity causes the valve to change its shape from a flat shape into a dome shape, thus allowing the valve to open up. Light control signals can be shone at different valve locations at different times in order to control the sequence of reagent release (see FIG. 4).

Now refers to FIG. 6A and FIG. 6C, the top part 68 can be made of acrylic, polycarbonate or similar kind of plastic materials. It may be transparent as to allow the user to observe the status of fluid inside the microfluidic chip 24. The plastic parts can be manufactured by plastic injection process associated with other process such as hot embossing and micro machining method. The top part 68 includes a plurality of micro grooves 66 in a corresponding plurality of reservoirs 80, wherein at least one reservoir is configured to receive the sample from the top and at least one reservoir is configured to hold at least one reagent for facilitating the reaction or interaction between the analyte interacting molecules and analyte. As such, the detection of analyte can be facilitated. The reagent held in the at least one reservoir is selected from the group consisting of washing buffer and blocking buffer. In one embodiment, the washing buffer is Phosphate-buffered saline (PBS) and the blocking buffer is PBS and Bovine serum albumin (BSA). The sample is driven from the microfluidic chip 24 to the diagnostic chip 26 for analyte reaction/interaction on the diagnostic chip 26. In each of the reservoir 80, at least one micro fluidic channel 86 is located beneath the micro grooves 66 at the interface between the top part 68 and bottom part 70 as shown in FIG. 6A and FIG. 6C. The reagent and the sample are driven from the microfluidic chip 24 to the diagnostic chip 26 through the micro fluidic channel 86 and then to channel opening.

Each of the reservoir 80 is integrated with a micro-pump which is constructed with small amount of hydro gel 82 placed therein (See FIG. 6C). The hydro gels 82 are in contact with electrical conductive circuit traces 84 incorporated onto the built material of the bottom part 70. These micro-pumps are operated by electrical current, which are supplied through electrical conductive circuit traces 84. These micro-pumps push the sample and reagent through the micro fluidic channels 86 whereby the sample and the reagent are transported to the channel opening by expanding and contracting the hydro gels 82. The expending and contracting of the hydro gels 82 are controlled by the microfluidic cartridge operating unit 41 of the microfluidic cartridge driver unit 30 of the diagnostic apparatus 20 by sending signals and power through the connection between electric connector 44 and the electric connecting interface, which is also in electrical connection with the electrical conductive circuit traces 84. The pumps are encapsulated so that it can avoid contamination and cross-contamination issues. In one example embodiment, the volume of each reservoir 80 is in a range of 20-150 μl. In one example embodiment, the number of reservoirs in one microfluidic cartridge is 5-12. For the sake of clarity, “mixed sample and reagent” is synonymous with “mixture of sample and reagent”, and refers to the mixture of sample and reagent formed by the steps described above.

In one example embodiment, a removable cap is provided at the opening for sample introduction to prevent leakage or evaporation of the samples (see FIG. 6B).

In one example embodiment, the expending and contracting of the hydro gels 82 are further controlled by the fluid control component 45 which receives signals from the control unit and power from the power source (see FIG. 4). The fluid control component 45 irradiates light onto a specific area of the plastic film on the microfluidic chip upon receiving signals. The irradiated light can change the viscosity of the hydro gels and thereby help pushing the sample and reagent through the micro fluidic channels by the micro-pumps.

Each electrical connector 44 on the bottom of the microfluidic cartridge 22 is associated with a specific reservoir. When connected, electric pulses pass through the electrical connector 44 and electrolyze the hydro gel in that specific reservoir. Oxygen and hydrogen are produced from the electrolysis process and these gases expand to push the fluid inside the reservoir out of the reservoir. The valve of the reservoir outlet is sealed by the plastic film but upon irradiation, the valve opens up and allow the reagent in the reservoir to be pushed through to the channel/the next reservoir (depending on where the outlet is connected to). The flow rate of the reagent is controlled by the electric pulse sequence that is passed to the electrical connectors 44 at the bottom of the chip.

The diagnostic chip 26 can be made of glass, silicon or plastic and is fixed to the microfluidic chip 24. The bottom surface (i.e. the surface facing the channel opening) of the diagnostic chip 26, which is pre-coated with an array of detection spots that can react/interact with the analyte present in a sample to generate at least one signal under certain condition (e.g. generating fluorescent signal(s) when radiated by a laser light at certain wavelength), is disposed toward and in fluid communication with the channel opening. In one embodiment, the detection spots each include at least one analyte interacting molecule that reacts/interacts with at least one analyte. In one specific embodiment, the analyte interacting molecule is a particular protein or peptide that binds with at least one particular virus/bacteria that is in its intact state or in portion suitable for being detected (e.g. an antigen). The array of detection spots is located within 1-15 millimeter (mm) around the channel opening such that the mixed sample and reagent can spread through the array when it is pumped out of the channel opening. The bottom surface of the diagnostic chip 26 facing towards the microfluidic chip 24 is first coated with a first coating for immobilizing the later coated detection spots without modifying the configuration of the detection spots (e.g. keeping the binding sites of the analyte interacting molecule included in the detection spots to be analyte(s) accessible). The first coating should also create a hydrophilic environment for the reaction/interaction of analyte to take place. It is optimized to minimize nonspecific reaction/interaction thus reduce background noise signal in the instant apparatus. Once the first coating is done, detection spots are deposited on the bottom surface of the diagnostic chip 26 in a pre-defined pattern (e.g. an array). A drop-on-demand method is chosen to disperse them onto the diagnostic chip 26. In one embodiment, the drop-on-demand method can be performed by a microarray printer. The diagnostic chip 26 with the mixed reagent and sample (which may include the analyte) reacted/interacted thereon can be detached from the microfluidic chip 24 and be placed to the diagnostic chip holder 58 for further analysis by the optical inspection unit 32. The mixed sample and reagent on the diagnostic chip 26 may be dried before or after the diagnostic chip 26 being detached from the microfluidic chip 24.

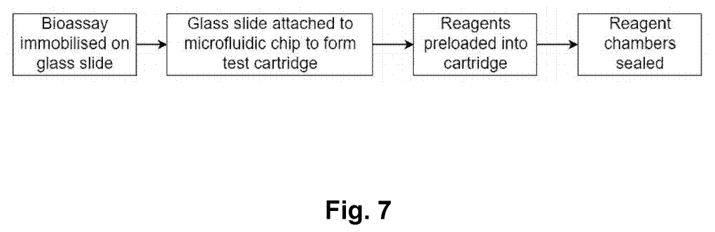

In one example embodiment, the microfluidic cartridge 22 (test cartridge) consists of (1) a bioassay printed on a diagnostic chip 26 and (2) a microfluidic chip 24 preloaded with reagents required for the microfluidic cartridge 22 to function properly. The production sequence for the microfluidic cartridge 22 is shown in FIG. 7. First, the bioassay is immobilized onto the diagnostic chip 26. Second, the diagnostic chip 26 is attached or fixed to the microfluidic chip 24 to form the microfluidic cartridge 22. Then, one or more reagents are pre-loaded into the one or more reservoirs of the microfluidic cartridge 22. Finally, the reagent chambers are sealed, for example, with a plastic film.

In one example embodiment, the bioassay can be based on immunoassay or any other type of bio-detection system. The bioassay consists of one or more positive control and negative control. Each bioassay can detect one disease at a time or can detect a plurality of diseases simultaneously. Unlike most near-patient tests, no external positive or negative control run is required before running the microfluidic cartridge 22.

In one example embodiment, the diagnostic chip 26 and the microfluidic chip 24 are pre-fixed during the manufacturing process and do not separate during the reaction and detection steps. The diagnostic chip 26 is pre-attached onto the microfluidic chip 24. This example embodiment eliminates errors cause when attaching the diagnosing chip 26 to the microfluidic chip 24 by the user or any other induced inaccuracies. This example embodiment eliminates the detachment step, eliminating or at least significantly reducing the risk of (1) reagent leakage leading to contamination or (2) the diagnostic chip snapping and cutting the user.

In one example embodiment, all reagents are preloaded into the microfluidic chip 24 and sealed during manufacturing process. Consider that near-patient tests are often performed by trained layman, preloading reagents eliminates chances of human error such as loading reagents into the wrong slot, incorrect use of pipette, adding wrong volume of reagents, and spillage of reagents during loading process. Preloading reagents also minimizes user contact with chemicals. The user-friendly microfluidic cartridge eliminates all preparation processes related to reagent loading thus minimizes chances of human error and cut preparation time by five minutes.

In one example embodiment, the shape of the microfluidic cartridge is configured such that there is only one possible way to fit or insert the microfluidic cartridge 22 and thus the diagnostic chip 26 into the microfluidic chamber 42 of the tray 52. The microfluidic cartridge 22 is secured in the desired position and orientation. In one exemplary embodiment, the steps of coating process and the deposition of detection spots containing e.g. antigen of the H7N9 influenza virus on the surface of the diagnostic chip 26 are shown in FIG. 8 and are detailed below: For the cleaning step 88: The diagnostic chip 26 of glass material is partly immersed in a 250 milliliter (ml) beaker containing acetone. Ultra-sonication is then performed for 5 minutes (mins) so as to clean the immersed part of the diagnostic chip 26. Diagnostic chip 26 is then transferred with forceps to another 250 ml beaker containing ethanol. Ultra-sonication is then performed again for 5 mins.

For the hydroxylation step 90: Seventy-five (75) ml of 95% sulfuric acid is transferred into a 250 ml beaker. Twenty-five (25) ml of 34.5% volume to volume (v/v) hydrogen peroxide is then pipetted to the same beaker, so that the final concentration of hydrogen peroxide is 8.63%, and that the resultant ratio between the volume of the concentrated sulfuric acid and the 34.5% hydrogen peroxide (piranha solution) is 1:3 v/v. Consequently, the diagnostic chip 26 from the cleaning step 88 is then partly immersed in above solution at room temperature for 2 hours (hrs). The treated diagnostic chip 26 is then picked up from the Piranha solution with forceps and is rinsed with ultrapure water using a wash bottle for 5 mins. The piranha solution is discarded into a waste bottle. Next, the treated diagnostic chip 26 is transferred with forceps to a 250 ml beaker containing 95% absolute ethanol. Ultra-sonication is then performed for 5 mins. Such step for the treated diagnostic chip 26 is then repeated in another 250 ml beaker containing purified water for one more time. For the acidification step 92: Twenty-five (25) ml of hydrochloric acid is transferred to a 50 ml reaction tube. Twenty-five (25) ml of ethanol is then added to the same tube. The diagnostic chip 26 from the hydroxylation step 90 is then transferred with forceps to the above solution and is reacted at 37 degree Celsius (° C.) for 3 hrs. The treated diagnostic chip 26 is then picked up from the solution with forceps and is rinsed with ultrapure water using a wash bottle for 5 mins. The solution is discarded into a waste bottle. Consequently, the washed diagnostic chip 26 is then transferred with forceps to a 250 ml beaker containing 95% absolute ethanol. Ultra-sonication is then performed for 5 mins.

The diagnostic chip 26 is then transferred with forceps to another 250 ml beaker containing purified water. Ultra-sonication is then performed again for 5 mins. After that, the treated diagnostic chip 26 is then transferred with forceps to a 250 ml beaker and is incubated in an oven for drying at 60° C. for 30 mins, before proceeding to the amination step 94 as described below.

For the amination step 94: Six point six hundred and forty one (6.641) gram (g) of (3-Aminopropyl) triethoxysilane (APTES) (moisture sensitive) at room temperature is pipetted to a 50 ml reaction tube (first use). Forty-three (43) ml of ethanol is then pipetted to the same tube. Next, 0.1 ml of acetic acid is then added to the same tube. The treated diagnostic chip 26 from acidification step 92 is then transferred with forceps to the above solution is reacted at 50° C. for 24 hrs. Consequently, the diagnostic chip 26 is then transferred with forceps to a 250 ml beaker containing 95% absolute ethanol. Ultra-sonication is then performed for 5 mins. The diagnostic chip 26 is then transferred with forceps to another 250 ml beaker containing purified water, and ultrasonication is then performed again for 5 mins. After that, the treated diagnostic chip 26 is then transferred with forceps to a 250 ml beaker and is incubated in an oven for drying at 120° C. for 30 mins.

For the addition step 96—adding aldehyde group: twenty-five percent glutaraldehyde is prepared each in 50 ml reaction tube. The treated diagnostic chip 26 from the amination step 94 is then transferred with forceps to the above solution and is reacted at room temperature for 24 hrs. Consequently, the diagnostic chip 26 is then transferred with forceps to a 250 ml beaker containing 95% absolute ethanol. Ultra-sonication is then performed for 5 mins. The diagnostic chip 26 is then transferred with forceps to another 250 ml beaker containing purified water and ultra-sonication is then performed again for 5 mins. Such step is then repeated in another 250 ml beaker containing purified water for one more time. Next, the treated diagnostic chip 26 is then transferred with forceps to a 250 ml beaker and is incubated in an oven for drying at 60° C. for 30 mins.

In an alternative embodiment, the diagnostic chip 26 is rinsed with deionized water, and is then ultra-sonicated for 5 mins in a 1:3 volume to volume (v/v) cleaning detergent: deionized water mixture. The cleaned diagnostic chip 26 is subsequently immersed for 5 mins in deionized water (after decantation), and is finally immersed for 5 mins in acetone. The cleaned diagnostic chip 26 is then dried with compressed air. Next, 3-glycidoxypropyltrimethoxysilane is then dissolved in acetone and is mixed with collodion solution (10%, obtained from Wako) with a pipette. The diagnostic chip 26 is dipped into this mixture and is withdrawn from the mixture slowly. The diagnostic chip 26 is then dried in air and turned to a white film. The coated diagnostic chip 26 is further incubated at 80° C. for 1 hour. The diagnostic chip 26 will then be submerged in 20 ml of ethanol for 5 mins after the equilibration at room temperature, The diagnostic chip 26 is then rinsed thoroughly with water, and is subsequently rinsed with acetone and water. The diagnostic chip 26 turned transparent and could be stored at room temperature before use for e.g. the printing step 98 as described below.

For the printing step 98—printing of PBS buffer, H7N9 antigen or BSA on the diagnostic chip 26 coated with aldehyde group as described in step 96, or on the transparent diagnostic chip 26 obtained from the acidification step 92: For printing with PBS buffer, prepare ink preparation of 40% glycerol in 4 ml PBS and fill the same in a printer cartridge. For printing H7N9 antigen, prepare ink preparation of 0.1 ml H7N9 antigen (from SinoBiological, at 1 milligram (mg)/ml) and 40% glycerol in 1.5 ml PBS, and fill the mixture in a printer cartridge. For printing with BSA, prepare ink preparation with 1 ml of 1000 microgram (μg)/ml of BSA (from Thermo, Product number 23208) solution with 40% glycerol in 4 ml PBS, and fill the mixture in a printer cartridge. Next, the FUJIFILM Dimatrix Materials Printer (model number DMP-2831) is set up. The prepared cartridge is then fixed onto the print head (precaution: ensure that no air bubbles are observed in the solution especially those being trapped in the inlet flow channel. If not, finger-tap on the cartridge until the bubbles are removed from the channel). The solution drop dripping stability from the 16 nozzles is then verified. At least one nozzle with good conditions is also chosen for the dot printing onto the treated diagnostic chip 26 from the addition step 96. The H7N9 antigen or BSA dot in 200 μm is then printed on the treated diagnostic chip 26. The printed diagnostic chip 26 is then transferred in a petri dish with cover, and is then incubated in drying oven at 37° C. for 2 hrs in the drying step 100.

The printed side of the processed diagnostic chip 26 in glass material from the drying step 100 is then attached using adhesive 74 to the top part 68 of the microfluidic chip 24 loaded with sample for detection as aforesaid, and the microfluidic cartridge 22 will proceed with optical inspection by the optical inspection unit 32 after the reaction/interaction with sample containing analyte.

### EXAMPLE 3

### Testing Method

Another example embodiment provides an on-site diagnostic method and an operation of the diagnostic system. Reagent purposed for facilitating analyte detection is/are pre-loaded to separate reservoirs 80 and sealed during the manufacturing process. The reagent held in the at least one reservoir is selected from the group consisting of washing buffer such as PBS, blocking buffer such as bovine serum albumin (BSA), lysing buffer such as PBS, antigens, antibodies and fluorophores such as Fluorescein in PBS. In one embodiment, the washing buffer is PBS and the blocking buffer is PBS and BSA. In one example embodiment, the microfluidic cartridge includes 5-12 reservoirs for retaining the reagents or samples. In some embodiments, the reagent volume is between 20-200 ul. In some embodiments, the reagent volume is 50 uL. Reagents may be held in one or more reservoirs. For example, if one reservoir is not large enough to hold all the required volume of a particular reagent, additional reservoirs may be used for the same reagent.

The diagnostic chip 26 is pre-fixed to the to the microfluidic chip 24 during the manufacturing process. The microfluidic chip 24 is first loaded with an appropriate amount of sample (e.g. a blood serum sample, a nasal swap or a nasopharyngeal swab) that may contain analyte by dispensing the sample into the reservoirs 80 in a loading step. In one example embodiment, the sample volume is between 20-200 ul. The diagnostic chip 26 surface having the array of the detection spots and the first coating faces toward the channel opening and the microfluidic chip 24. The array of the detection spots and the first coating will be located within the 1-15 mm vicinity of the channel opening. The microfluidic cartridge 22 is then docked to the cartridge chamber 42 of the microfluidic cartridge driver unit 30 in docking step by putting an electric connecting interface through the microfluidic cartridge receiving hole, such that the electric connecting interface will be in contact with electrical connectors 44. In one example embodiment, the microfluidic chip of the microfluidic cartridge will be directly underneath the fluid control component 45. Then the identity of the microfluidic cartridge is read by a reader upon receiving signal from the switch, wherein said signal is sent when the tray is inserted into the docked position along the tray rail. In one example embodiment, the microfluidic cartridge is identified by the 2D code and is read by the barcode reader. The microfluidic cartridge is identified by the control unit and the required predetermined sequence is automatically selected. The mixed sample and reagent are spread across the array of detection spots in the spreading analyte step. This is done by flowing the sample and reagent from the reservoirs 80 through the microfluidic channels 86 of the microfluidic chip 24 to the channel opening. Upon receiving an electrical current and signals by the electric connecting interface from the microfluidic cartridge chamber via electrical connectors 44, the micro-pumps drive the sample through the microfluidic channels 86 at the time, speed and sequences as instructed by the microprocessor of the control unit 28. The mixed sample and reagent (the sample is mixed with the reagent while flowing through the microfluidic channels 86 of the microfluidic chip 24 as aforesaid) that exits the channel opening spreads across the bottom surface of the diagnostic chip 26. Any gas bubbles in the reservoir 80 will be removed through the microporous membrane 76 located at the bottom part 70 of the microfluidic chip 24 as the sample passes through it from the microfluidic channels 86. The area where the mixed sample and reagent spread covers the place where the array of detection spots locates such that the analyte can react/interact with the analyte interacting molecule in the detection spots. In one embodiment, the spreading analyte step can further include the step of further driving the microfluidic chip 24 to spread a second auxiliary reagent, which is located at one of the reservoirs 80, by flowing through the microfluidic channels 86 of the microfluidic chip 24 to the diagnostic chip 26 for attaching a secondary molecule for facilitating the detection of reacted or interacted analyte after the mixed sample and reagent is spread on the array of detection spots. When pumping and the analyte reaction/interaction are stopped, the microfluidic cartridge 22 remains the same position in the cartridge chamber 42 of the microfluidic cartridge driver unit 30. In one example embodiment, the spreading analyte step can further include the step of further driving the fluid control component 45 to change the viscosity of at least one specific part of the microfluidic cartridge by controlling irradiating light on specific parts of the microfluidic chip and opening a valve which facilitate the fluid movements. The mixed sample and reagent on the diagnostic chip 26 may be dried. After that, an analyzing step can begin. The diagnostic chip 26 of the microfluidic cartridge is located underneath the optical sensor 48 and does not separate from the microfluidic cartridge after the spreading analyte step. Upon the receiving of the starting signal from the microprocessor, light beam from the illumination system 50 (e.g. a laser beam) is then directed onto the diagnostic chip 26 to generate at least one signal (if the mixed sample and reagent contains the analyte) detectable by the optical sensor 48. In one embodiment, the at least one signal includes fluorescence signal is generated when the diagnostic chip 26 radiated by the suitable light at suitable wavelength (e.g. 488 nm). The signal collected will be converted into digital data which will then be transferred to and analyzed in by the microprocessor of the control unit 28 to determine the presence of the analyte quantitatively or qualitatively. The result will be shown on the display unit 34 of the apparatus in relatively short period of time (fast) (e.g. in a range of 10-25 mins).

In one example embodiment, there are a number of steps in which manual assembly and disassembly of the microfluidic cartridge is required, alongside the manual selection of test program required.

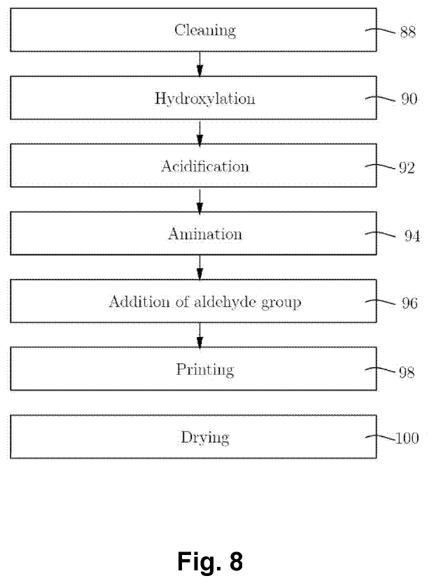

In one example embodiment, the present invention has been designed to have minimal human involvement which minimizes the chances for human error. With all reagents preloaded into the microfluidic cartridge and sealed, only the sample chamber inlet is exposed and is the only obvious inlet for where the sample should be loaded. This design minimizes the chance for the user to load the sample into a wrong chamber. As the microfluidic cartridge is inserted into the apparatus, the barcode reader scans the data matrix on the microfluidic cartridge and either rejects the cartridge if it has already been used, or accepts the microfluidic cartridge and automatically selects the correct test program. This feature prevents any used microfluidic cartridge to be accidentally re-used and prevents the user from making mistakes when selecting the test program on the diagnostic platform. The software embedded in the diagnostic platforms analyzes and shows the test results on the screen which eliminate any chances of human misinterpretation when reading the results. FIG. 9 shows an example report detail for displaying the test results on the screen. The report detail shows a direct results interpretation that both positive controls and negative controls are valid and the target FluA is detected.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the apparatus can further include at least one USB port or any other data communication means to allow the operation of common communication protocols of data transfer. The display unit is equipped in the apparatus for human interface. The display unit 34 is a high resolution color display that can be either a liquid-crystal display (LCD), Organic Light-Emitting Diode (OLED) or other kind of display. The display unit can be incorporated with a touch screen panel; therefore, it can receive command from the touch of human fingers. The display unit is connected with the control unit 28. However, the way it displays, the content being displayed is made by the graphic user interface.

An exemplary microfluidic chip that can be used can be the microfluidic chip disclosed in German patent application numbers DE102010061910.8, DE102010061909.4 and DE502007004366.4.

In yet another alternative embodiment, instead of using the at least one laser beam, at least one light beam can be used generate at least one signal for the analysis. The illumination system 50 in this alternative embodiment emits at least one light beam with at least one predetermined wavelength on the diagnostic chip 26. The illumination system 50 comprises a light-emitting diode (LED), at least one filter and at least one dichroic mirror.

In another embodiment, the illumination system 50 can have more than one diode laser or more than one LED.

In yet another embodiment, the camera 62 of the inspection unit 32 can be a digital high resolution camera 62, in which the sensor is selected from a group of Complementary metal-oxide-semiconductor (CMOS) sensor and Charge-coupled device (CCD) sensor. The megapixels of the image sensor of the digital high resolution camera 62 is in a range of 1.0 Megapixels to 30 Megapixels.

In yet other embodiment, the diagnostic apparatus 20 can include multiple microfluidic cartridge driver units 30 and multiple optical inspection units 32 so that the multiple analyses/diagnoses can be run at the same time. While we have described a number of embodiments of this invention, it should be understood that these examples may be altered to provide other embodiments of the invention. Therefore, the scope of this invention is to be defined by the following claims rather than by the specific embodiments provided herein.

### NUMBERED EMBODIMENTS

The invention is further described with reference to the following numbered embodiments.
1. An apparatus for detecting at least one analyte from a sample comprising:
a microfluidic cartridge driver unit comprising:
a tray comprising a cartridge chamber configured to receive a microfluidic cartridge configured for a reaction, wherein the reaction comprises interacting or reacting with said analyte; and
   a microfluidic cartridge operating unit comprising at least one electrical connector configured to connect with said microfluidic cartridge for electrical connection therewith;
   an optical inspection unit configured for analyte detection, wherein the analyte detection comprises detecting at least one signal generated from said microfluidic cartridge due to the presence of said analyte at a predetermined condition, said optical inspection unit comprising:
      an illumination system configured to deliver light to said microfluidic cartridge, thereby providing said predetermined condition; an optical sensor configured to detect said at least one signal;
      at least one filter configured to filter out any undesired wavelength or noise from light produced from the illumination system; and
a control unit configured to control the quantitative and qualitative analysis, interfacing, and storage of said at least one signal obtained from said optical inspection unit, and to control and monitor the operation of said apparatus;
wherein the tray is configured such that the reaction and the analyte detection are performed on the same microfluidic cartridge in the same location or position.
2. The apparatus of embodiment 1, wherein the tray is configured such that the tray is slidably removable for receiving said microfluidic cartridge in a docked position.

3. The apparatus of embodiment 2, wherein the tray further comprises an anchoring system to secure said microfluidic cartridge.

4. The apparatus of embodiment 3, wherein the anchoring system comprises two anchoring clips positioned orthogonal to each other.

5. The apparatus of embodiment 3 or embodiment 4, wherein the anchoring system is configured such that the tolerance of the position of the microfluidic cartridge is below 0.1 mm.

6. The apparatus of any one of embodiments 1-5, wherein the optical inspection unit further comprises a reader to identify the identity of the microfluidic cartridge and the required predetermined sequence.

7. The apparatus of embodiment 6, further comprising a switch.

8. The apparatus of any one of claims 1-7, wherein the optical inspection unit further comprises at least one lens for focusing an image.

9. The apparatus of any one of embodiments 1-8, wherein said microfluidic cartridge operating unit further comprises at least one fluid control component configured to facilitate at least one fluid movement within the microfluidic cartridge.

10. The apparatus of any one of embodiments 1-9, wherein said optical sensor comprises a camera and at least one objective lens.

11.The apparatus of embodiment 9, wherein said optical sensor is selected from the group consisting of Complementary metal-oxide-semiconductor (CMOS) sensor and Charge-coupled device (CCD) sensor.

12. The apparatus of any one of embodiments 1-11, wherein said illumination system comprises at least one light source and optionally at least one light focusing lens.

13. The apparatus of any one of embodiments 12, wherein the light source comprises at least one light tube.

14. The apparatus of any one of embodiments 1-13, wherein said analyte is influenza virus antigen and said wavelength of said diode laser is 488 nm.

15. The apparatus of any one of embodiments 1-14, wherein the control unit is capable of controlling said microfluidic cartridge driver unit.

16. The apparatus of any one of embodiments 1-15, wherein said apparatus comprises a power supply comprising a built-in rechargeable battery.

### EXAMPLE 4

For the following examples, the term “microfluidic portion” refers to “microfluidic chip” as used in the previous examples; the term “microvalve” refers to “valve” as used in the previous examples; the term “diagnostic portion” refers to “diagnostic chip” as used in the previous examples; the term “rail component” refers to “rail system” as used in the previous examples; the term “optical unit” refers to “optical inspection unit” as used in the previous examples; the term “illumination component” refers to “illumination system” as used in the previous examples; the term “sensor component” refers to “optical sensor” as used in the previous examples; the term “cartridge driver unit” refers to “fluid control component” as used in the previous examples.; the term “tray cover” refers to “tray board” as used in the previous examples; the term “microchannels” refers to “microfluidic channels” as used in the previous examples.

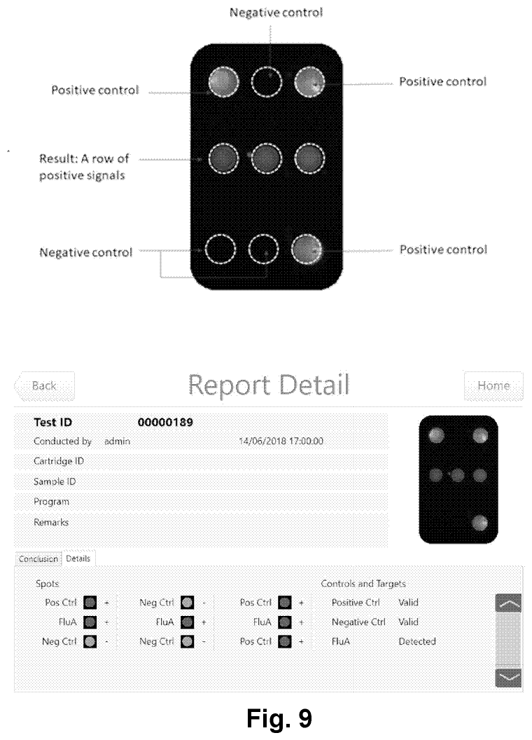

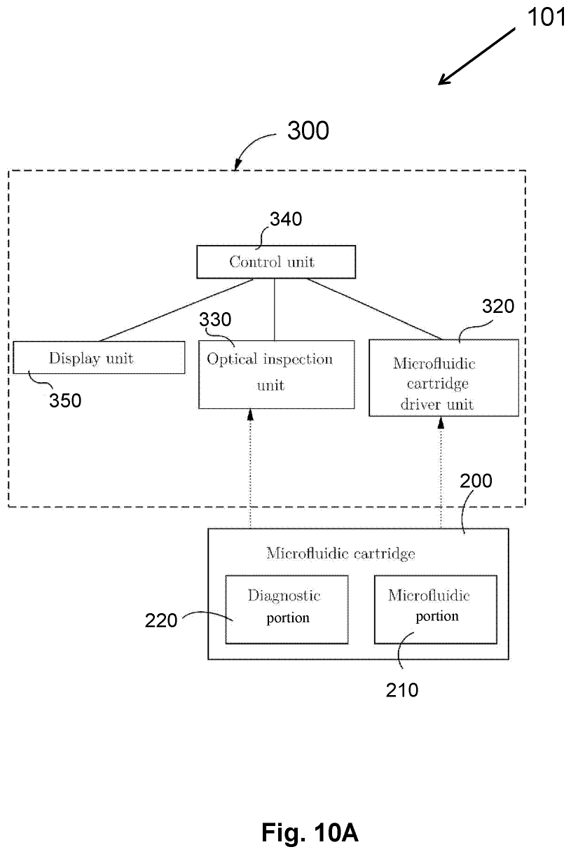

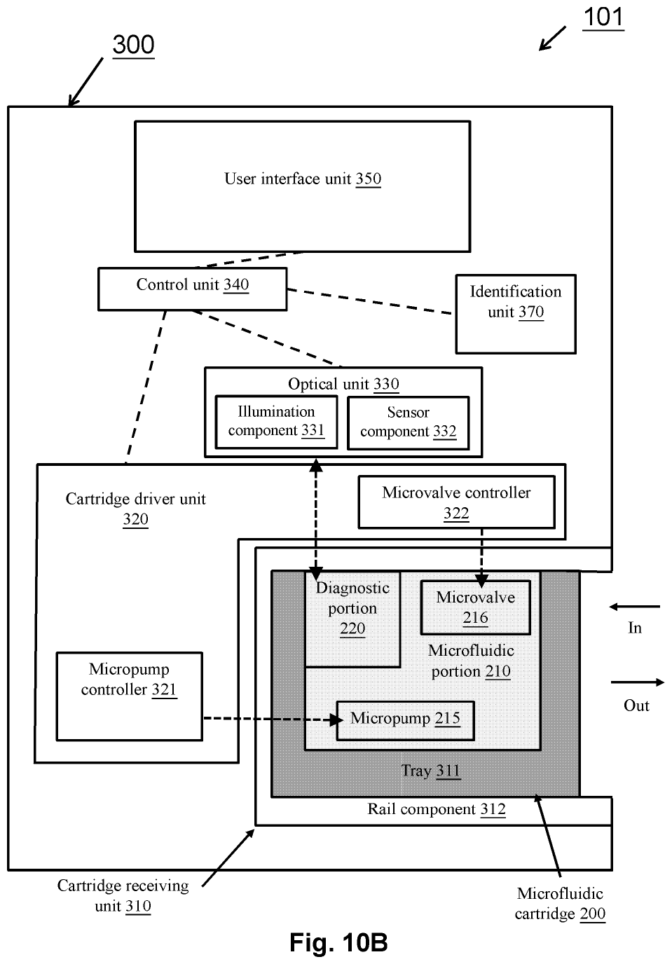

Referring now to FIG. 10A, a diagnostic system 101 comprising a portable diagnostic apparatus 300 and a microfluidic cartridge 200 which operates with the portable diagnostic apparatus 300 is shown. The microfluidic cartridge 200 which includes a microfluidic portion 210 and a diagnostic portion 220 is configured to collect and manipulate at least one sample, which may include at least one analyte. The microfluidic cartridge 200 also receives and/or holds at least one reagent. The portable diagnostic apparatus 300, which is a portable, hand carriable and compact device, includes, a microfluidic cartridge driver unit 320, an optical unit 330 and optionally a control unit 340. In this example, the optical unit 330 is an optical inspection unit. In some embodiments, the portable diagnostic apparatus 300 optionally comprises a user interface unit 350 for interfacing with users. In this example, the user interface unit 350 is a display unit. The control unit 340 controls and is connected to the microfluidic cartridge driver unit 320, the optical unit 330 and the user interface unit 350. The microfluidic cartridge driver unit 320 is configured to receive and drive the microfluidic cartridge 200 such that the sample collected and the reagent run through the microfluidic portion 210 and the diagnostic portion 220 in a predetermined sequence. After the reaction is done in the predetermined sequence, the microfluidic cartridge driver unit 320 also allows inspection or analysis of the diagnostic portion 220 to occur on the same microfluidic cartridge in the same location or position. The optical unit 330 is configured to inspect the diagnostic portion 220 on the same microfluidic cartridge 200 in the same location where the reaction is done for analyzing the presence of analyte. The user interface unit 350 is configured to display relevant information including analyzed/diagnostic results to users.

### EXAMPLE 5

Referring now to FIG. 10B, which shows another example embodiment of a diagnostic system 101 comprising a (1) portable diagnostic apparatus 300 and (2) a microfluidic cartridge 200, which operates with the portable diagnostic apparatus 300. In this example, the microfluidic cartridge 200 includes a microfluidic portion 210 and a diagnostic portion 220. The microfluidic portion 210 further comprises at least one microvalve 216 and at least one micropump 215. The portable diagnostic apparatus 300, which is a portable, hand carriable and compact device, includes a control unit 340, a microfluidic cartridge driver unit 320, an optical unit 330 and a cartridge receiving unit 310. In this example, the portable diagnostic apparatus 300 further comprises an identification unit 370 to recognize the identity of the microfluidic cartridge 200. FIG. 10B shows that the control unit 340, microfluidic cartridge driver unit 320, optical unit 330, user interface unit 350, a cartridge receiving unit 310 and power supply unit 360 (not shown) are enclosed in a self-contained diagnostic apparatus. In this example, the control unit 340 is electrically connected with the cartridge receiving unit 310, the microfluidic cartridge driver unit 320, optical unit 330 and user interface unit 350 to ensure the microfluidic cartridge 200 is in a desired designated area for analysis, to control the flow of fluid within the microfluidic cartridge 200, control the quantitative and qualitative analysis, interfacing, and storage of said at least one signal obtained from said optical inspection unit, and to control and monitor the operation of said apparatus. In some example embodiments, the cartridge receiving unit 310 comprises a tray 311 and a rail component 312. In this example, the tray 311 is slidable from the cartridge receiving unit 310 and comprises a cartridge chamber for receiving the microfluidic cartridge 200 so that a user may pull out at least part of the tray 311 from the portable diagnostic apparatus 300, install a microfluidic cartridge 200 onto the cartridge chamber of the tray 311 and insert the tray 311 into the rail component 312, to ensure that the microfluidic cartridge is being positioned at the desired, designated area of the portable diagnostic apparatus 300. In another example embodiment, the tray 311 is slidably removeable from the cartridge receiving unit 310 so that a user may completely take out the tray 311 from the apparatus. The rail component 312 is fixedly attached to the portable diagnostic apparatus 300 and configured to receive the tray 311 with a microfluidic cartridge 200 inserted therein. In one example embodiment, the portable diagnostic apparatus 300 includes a microfluidic cartridge receiving cavity (not shown) on its front panel for receiving the microfluidic cartridge 200. The portable diagnostic apparatus 300 may also be enclosed in a self-contained housing.

### EXAMPLE 6

### Microfluidic Cartridge

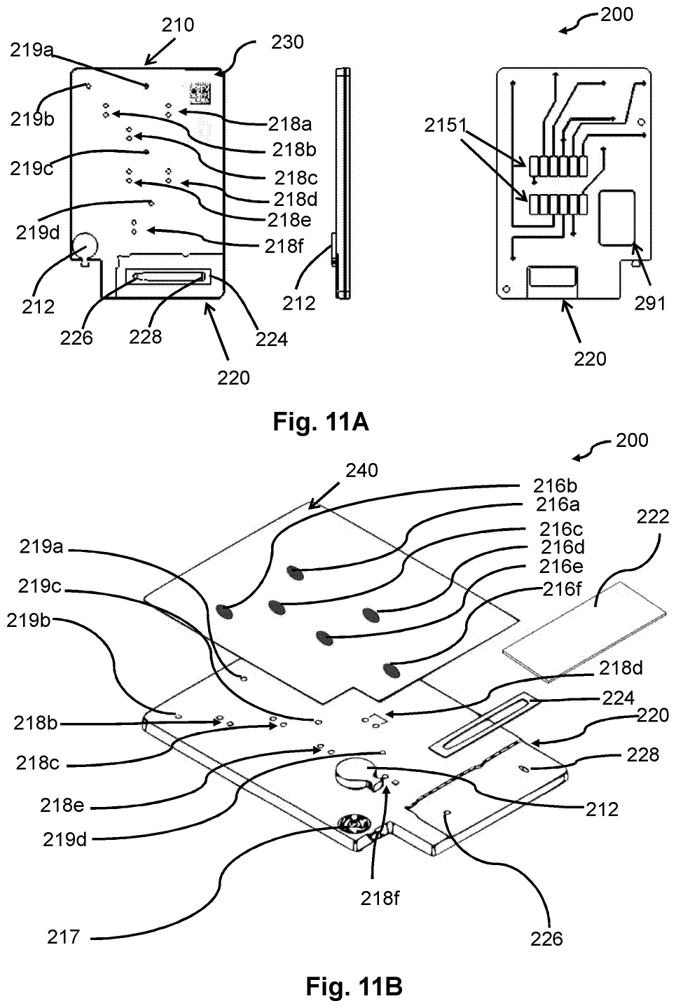

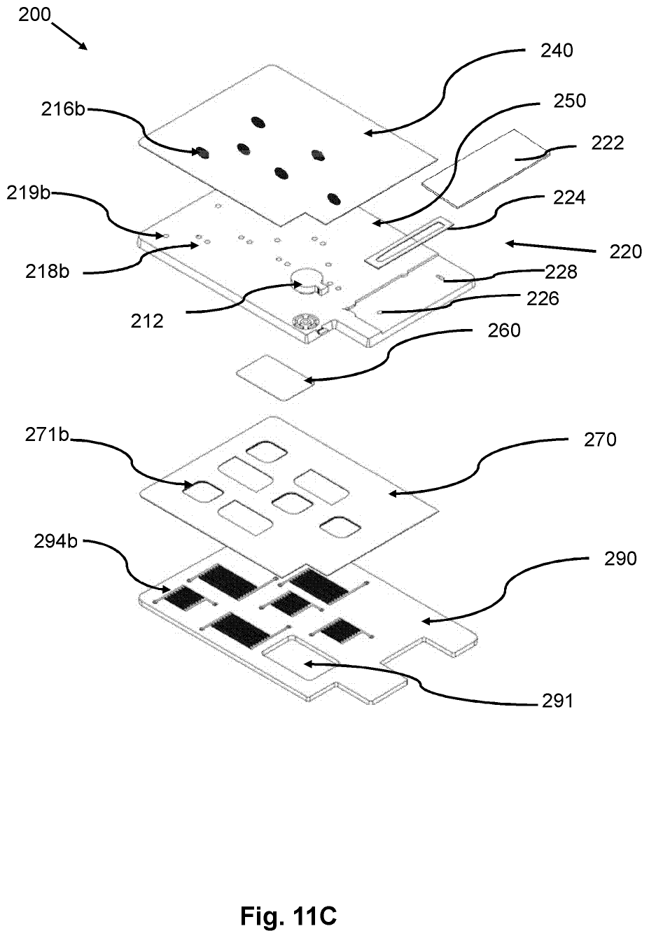

Referring now to FIG. 11A, which shows an example embodiment of the front view (LEFT), side view (MIDDLE) and the back view (RIGHT) of a microfluidic cartridge 200. The microfluidic cartridge 200 which includes a microfluidic portion 210 and a diagnostic portion 220 is configured to collect and manipulate at least one sample, which may include at least one analyte. In this example, the diagnostic portion 220 includes a diagnostic chip (not shown) and an adhesive tape 224. The microfluidic cartridge 200 may also include at least one reactant disposed on the diagnostic chip (not shown). In this example, the adhesive tape 224 is fixedly attached the diagnostic chip onto the diagnostic portion 220. Adhesive tape 224 has a thickness and is made of plastic materials such as polycarbonate coated by an adhesive material on both opposing sides. The adhesive tape 224 has a confined area therein, forming a diagnostic chamber for the pre-loaded reactant and a reaction chamber for fluid communication between the diagnostic portion 220 and the microfluidic portion 210. In this example, an inlet 226 for the fluids such as sample and reagents from the microfluidic portion and an outlet 228 to a reservoir for waste are disposed within the confined area of the adhesive tape 224. In some embodiments, the at least one reactant is pre-supplied on the diagnostic chip 220, that is, the reactant is pre-loaded during the manufacturing process, saving the efforts, resources and time for a user to prepare the diagnostic chip. The microfluidic cartridge may include a combination of microvalves, microchannels, reservoirs, inlets and outlets etc. positioned in various configurations to allow various geometries of the fluid delivery. In some embodiments, the microfluidic cartridge may comprise at least one sample reservoir and at least one reagent reservoir. Additionally, the microfluidic cartridge may be made with a built-in waste reservoir to handle fluids after analysis such as bio-hazard materials. In the implementation shown, it has a dimension smaller than a credit card with a thickness of 1-10 mm and the dimension is around 30-60 mm×50-80 mm. In another example embodiment, the thickness of the microfluidic cartridge can be around 5 mm and the dimension can be around 40 mm×60 mm. The inlet of the sample reservoir is capped by a sample cap 212, allowing user to open the sample cap 212 upon applying a sample and keeping the sample reservoir in a closed state to avoid potential contamination. The cover layer, microvalve membrane 240, has been removed to show the inlets and outlets 218a, 218b, 218c, 218d, 218e and 218f. In this example, 219a, 219b, 219c and 219d are inlets for reagents respectively, allowing different reagents being introduced into each of the corresponding reagent reservoirs 213 (not shown) during manufacturing process, saving the efforts, resources and time for a user to apply the appropriate reagents. 218a, 218b, 218c, 218d, 218e and 218f are the inlet/outlet junction pairs for the respective reagent reservoirs (not shown). The microfluidic cartridge 200 may also comprise an indicator showing the cartridge identity 230 of the microfluidic cartridge 200. In one example embodiment, the cartridge identity 230 may be fixedly attached to the top part. In another example embodiment, the cartridge identity 230 may be fixedly attached to the microvalve membrane 240. In this example, the cartridge identity 230 is shown as a unique serial number and a two-dimensional (2-D) bar code, which can be identified by an identification unit 370 (not shown). The microfluidic cartridge 200 includes an electrical connecting interface 2151 for receiving control signals and power provided through electrical connectors disposed on the cartridge chamber of the cartridge receiving unit (not shown).

FIG. 11A (RIGHT) shows the opposite (bottom) side of the microfluidic cartridge 200. The microfluidic chip 210 includes an electrical connecting interface 2151 for receiving control signals from the apparatus to provide power or electrical current to the microfluidic cartridge 200. FIG. 11A also shows the diagnostic portion 220 is at least partially transparent for detection of sample. The microfluidic cartridge 200 also includes a cavity 291 for the microporous membrane.

Referring now to FIG. 11B, the microvalve membrane 240, sample cap 212, diagnostic chip 222 and an adhesive tape 224 are blown out from the microfluidic cartridge 200 for better representation. In this example, the microvalve membrane 240 is a film having microvalves 216a, 216b, 216c, 216d, 216e and 216f. Microvalve membrane 240 may be attached to a top part of the microfluidic cartridge 200 by adhesive means known in the art. Microvalve membrane 240 covers and seals the microfluidic portion 210 of the microfluidic cartridge 200. In some embodiments, the microvalve membrane 240 (and the microvalves 216) are made of a material that expands and changes its shape in response to stimuli such as temperature. In some embodiments, only the microvalve 216 portions are made of a material that expands and changes its shape in response to stimuli such as temperature and the rest of the portion of the microvalve membrane 240 uses other suitable materials. In this example, the entire microvalve membrane 240 is made of parafilm. In yet some other embodiments, the microvalve membrane may be made of polyurethane and/or Nylon. Microvalves 216a, 216b, 216c, 216d, 216e and 216f are flat and seal the inlet/outlet junctions 218a (not shown), 218b, 218c, 218d, 218e and 218f, respectively, for the corresponding reagent reservoirs (not shown) in a normally closed state. When the microvalves are exposed to heat, the materials of the microvalves expand and change into a dome shape (i.e. an open state), allowing fluid communication between the inlet/outlet junctions 218a (not shown), 218b, 218c, 218d, 218e and 218f, respectively. In some example embodiments, the opening of the microvalves is irreversible, that is, the microvalves are single-use and cannot be closed again after opening. In yet another example embodiment, the opening of the microvalves is reversible. In some example embodiments, the microvalve membrane can be transparent or semi-transparent, allowing users to observe the flow of fluids in the microfluidic cartridge 200. In a further example embodiment, the microvalves area can be in dark colour such as black for better absorption of light (and thus heat) energy. In this example, the microvalves are represented as black spots on the microvalve membrane. In this example, a removable sample cap 212 is shaped to match the diameter of the inlet of the sample reservoir 211. The diagnostic portion 220 is in fluid communication with the microfluidic portion 210 through openings, inlet 226 and outlet 228, respectively. In this example, the reactant may be disposed on the diagnostic chip 222 within the confined area of the diagnostic chamber.

In some embodiments, the microfluidic cartridge 200 may comprise a microvalve membrane, a top part, at least one adhesive layer, a plurality of micropumps, a microporous membrane and a bottom part. Referring now to FIG. 11C, which shows an exploded view of the same example embodiment of FIG. 11A. For clarity, some of the same or similar elements in this microfluidic cartridge, only one of them will be annotated as an example. In this example, microfluidic cartridge 200 comprises a microvalve membrane 240 having a number of microvalves (such as 216b), a top part 250, an adhesive layer 270, a number of micropumps (not shown), a microporous membrane 260 and a bottom part 290, which assembled together as a single unit with adhesive materials or by welding process. Microvalve membrane 240 can be a thin film made of parafilm. In yet some other embodiments, the microvalve membrane 240 may be made of polyurethane and/or Nylon. Microvalve membrane 240 receives heat energy as control signals provided by the microvalve controller of the microfluidic cartridge driver unit (not shown; will be described later) to control the opening of the microvalves when in use. When sufficient light control signals are received at a particular microvalve location on the microvalve membrane 240, the microvalve changes viscosity. The change in viscosity causes the microvalve to change its shape from a flat shape into a dome shape, thus allowing the microvalve to open up. Light control signals can be shone at different microvalve locations at different times in order to control the sequence of reagent release. Top part 250 comprises one sample inlet 212, a number of reagent inlets (such as 219b), a number of pairs of inlet/outlet junctions (for example, 218b) and an adhesive tape 224. Adhesive tape 224 may be made of plastic such as acrylic, polycarbonate and it defines a diagnostic chamber within the diagnostic portion 220. Reagent inlets 219b forms a fluid introduction port in fluid communication with a reservoir (not shown), which is disposed on the opposite side of the top part 250. Adhesive layer 270 is a plastic film having a thickness and made of plastic materials such as polycarbonate coated by an adhesive material on both opposing sides, providing an adhesive force to join the top part 250 and bottom part 290 together. Adhesive layer 270 has a thickness and includes a number of grooves (such as 271b), which are cavities for accommodating the spaces of the reservoirs for reagents and sample. Each groove (such as 271b) has a position that corresponds to the respective position of the reservoir, allowing the reservoir to be in direct contact with the next layer (i.e., the bottom part 290). The substrate of bottom part 290 may be made of electrical insulated material such as plastic and resin materials. Bottom part 290 has a groove 291 for a microporous membrane 260 to be placed therein. Bottom part 290 also includes a number of electrical connecting interface (not shown) which is in electrical connection to the apparatus, and a number of electrical conductive traces (such as 294b). A space formed between each electrical conductive trace (such as 294b) and each corresponding groove (such as 271b) allows hydrogel being disposed within. Electrical connecting interface is electrically connected to the electrical conductive traces. Electrical conductive traces (such as 294b) may directly or indirect in contact with the hydrogel (not shown).

Referring still to FIG. 11C, top part 240 can be made of acrylic, polycarbonate or similar kind of plastic materials. It may be transparent or partially transparent as to allow the user to observe the status of fluid inside the microfluidic portion 210. The plastic parts can be manufactured by plastic injection process associated with other process such as hot embossing and micro machining method. The top part 250 includes a plurality of cavities such as 271b corresponding to plurality of reservoirs (not shown), wherein at least one reservoir is configured to receive the sample from the top part and at least one reservoir is configured to hold at least one reagent for facilitating the reaction or interaction between the analyte interacting molecules and analyte. As such, the detection of analyte can be facilitated. The reagent held in the at least one reservoir may be washing buffer or blocking buffer. The sample is driven from the microfluidic portion 210 to the diagnostic portion 220 for analyte reaction/interaction on the diagnostic chip 222. The reagent and the sample are driven from the microfluidic portion 210 to the diagnostic portion 220 through the micro fluidic channel (not shown) and then to inlet 226.

Each of the reservoir (not shown) is integrated with a micropump which is constructed with small amount of hydrogel placed therein (not shown). The hydrogels are in contact with electrical conductive traces (such as 294b) incorporated onto the built material of the bottom part 290. These micropumps are operated by electrical current, which are supplied through electrical conductive traces (such as 294b). These micropumps push the sample and reagent through the micro fluidic channels by expanding and contracting the hydrogels whereby the sample and the reagent are driven to the channel opening. The expanding and contracting of the hydrogels are controlled by the microfluidic cartridge driver unit 320 of the diagnostic apparatus by sending signals and power through the connection between electrical connecting interfaces (not shown, on the bottom opposing side of the bottom part) and the electrical conductive traces (such as 294b). In some embodiments, the hydrogels of the micropumps are encapsulated so that contamination and cross-contamination issues can be avoided. In yet some other embodiments, the hydrogels of the micropumps may be in direct contact with the fluids such as reagents or samples within the reservoirs. In some embodiments, the microfluidic cartridge further comprises a micropump membrane for sealing the hydrogel. Microvalve membrane also covers all reservoirs to prevent fluid leakage. The micropump membrane also helps pushing the fluid out of the reservoir by the action of the micropump of the microfluidic cartridge. The micropump membrane may include a groove for a microporous membrane, allowing the microporous membrane to be in direct contact with the ambient. In one example embodiment, the volume of each reservoir (not shown) is in a range of 20-150 μl. In another example embodiment, the volume of each reservoir (not shown) is in a range of 20-200 μl. In one example embodiment, the number of reservoirs in one microfluidic cartridge is 5-12. In one example embodiment, a removable sample cap 212 is provided at the opening for sample introduction to prevent leakage or evaporation of the samples (see FIG. 11C).

Each electrical conductive trace (such as 294b) of the microfluidic cartridge 200 is associated with a specific reservoir. When connected, electric pulses from the electrical connecting interface pass through the electrical conductive trace and electrolyze the hydrogel in that specific reservoir. Oxygen and hydrogen are produced from the electrolysis process and these gases expand to push the fluid inside the reservoir out of the reservoir. The valve of the reservoir outlet is sealed by the plastic film but upon irradiation, the valve opens up and allow the reagent in the reservoir to be pushed through to the microchannel/the next reservoir (depending on where the outlet is connected to). The flow rate of the reagent is controlled by the electric pulse sequence that is passed to the electrical conductive trace of the bottom part 290 of the microfluidic cartridge 200.

The diagnostic chip 222 can be made of glass, silicon or plastic and is fixed to the diagnostic portion. The bottom surface (i.e. the surface facing the channel opening) of the diagnostic chip 222, which is pre-coated with an array of detection spots that can react/interact with the analyte present in a sample to generate at least one signal under certain condition (e.g. generating fluorescent signal(s) when radiated by a laser light at certain wavelength), is disposed toward and in fluid communication with the channel opening. In one embodiment, the detection spots each include at least one analyte interacting molecule that reacts/interacts with at least one analyte. In one specific embodiment, the analyte interacting molecule is a particular protein or peptide that binds with at least one particular virus/bacteria that is in its intact state or in portion suitable for being detected (e.g. an antigen). The array of detection spots is located at the diagnostic chip 222 of the diagnostic portion such that the sample and reagent can spread through the array when they are pumped out of the inlet 226 (FIG. 11B). The bottom surface of the diagnostic chip 222 is first coated with a first coating for immobilizing the later coated detection spots without modifying the configuration of the detection spots (e.g. keeping the binding sites of the analyte interacting molecule included in the detection spots to be analyte(s) accessible). The first coating should also create a hydrophilic environment for the reaction/interaction of analyte to take place. It is optimized to minimize nonspecific reaction/interaction thus reduce background noise signal in the instant apparatus. Once the first coating is done, detection spots are deposited on the bottom surface of the diagnostic chip 222 in a pre-defined pattern (e.g. an array). A drop-on-demand method is chosen to disperse them onto the diagnostic chip 222. In one embodiment, the drop-on-demand method can be performed by a microarray printer. In some embodiments, the microfluidic cartridge is pre-supplied with at least one reagent and/or at least on reactant and it is disposable. In some embodiments, the microfluidic cartridge comprises a number of reservoirs for storing reagents, samples and waste. The reservoirs may be in fluid communication with the microchannels.

### EXAMPLE 7

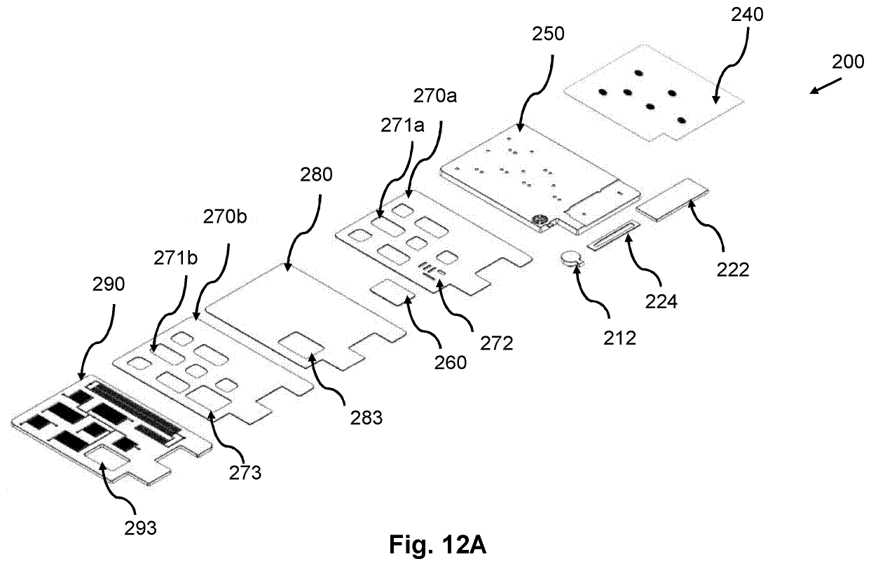

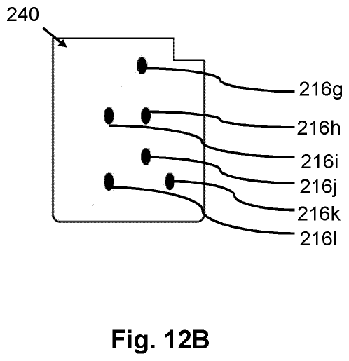

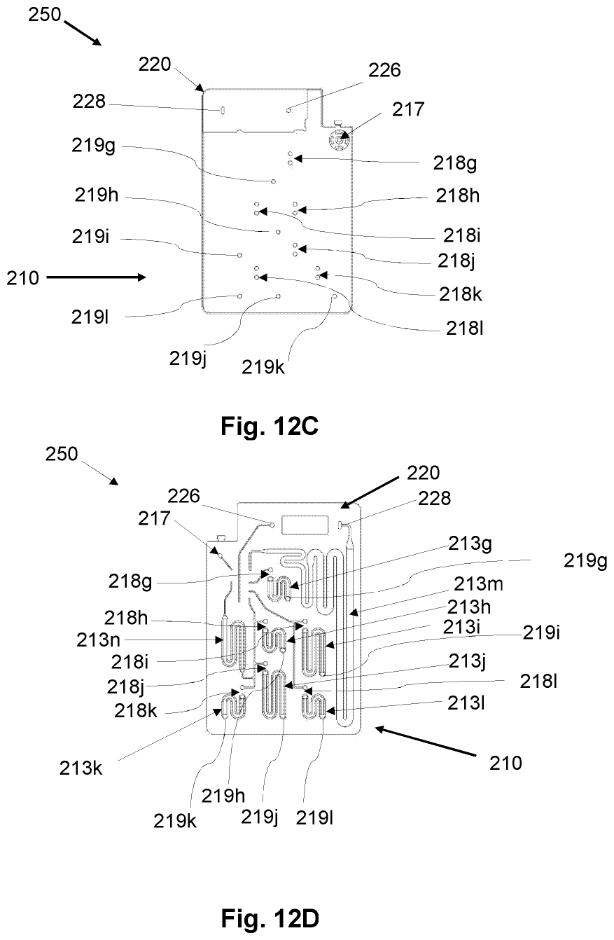

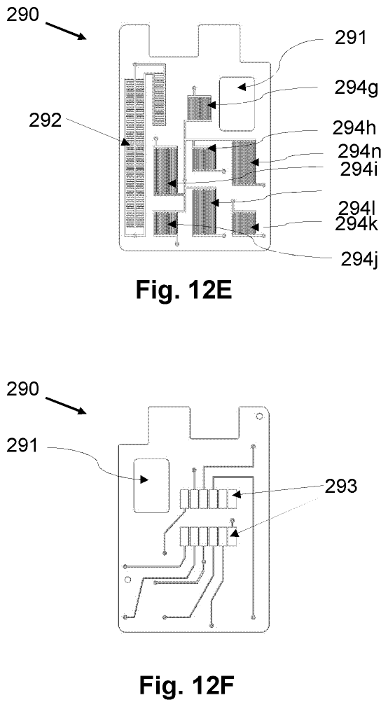

Referring now to FIG. 12A-12F, which show another example embodiment of the microfluidic cartridge 200. FIG. 12A shows an exploded diagram of the example embodiment of the microfluidic cartridge 200. The diagram shows how the microfluidic cartridge can be assembled using different layers of material. In this example embodiment, the microfluidic cartridge 200 is assembled with a microvalve membrane 240 on top, followed sequentially with a top part 250, a first adhesive layer 270a, a microporous membrane 260, micropump membrane 280, a second adhesive layer 270b, and a bottom layer 290. The top part 250 further comprises a microfluidic portion having a sample cap 212 and a diagnostic portion having an adhesive tape 224 and a diagnostic chip 222. The adhesive tape 224 is configured to form a diagnostic chamber (not shown) in the diagnostic portion 220 with the diagnostic chip 222. In some example embodiments, the microfluidic cartridge 200 includes a microfluidic portion 210 and a diagnostic portion 220, wherein the microfluidic portion 210 includes a number of reservoirs (not shown) capable of holding fluid therein, a number of microchannels (not shown) for fluid connection from the reservoirs to the diagnostic portion 220, a number of microvalves 216 operable between a closed state and an open state for sealing and opening the microchannel connections respectively; and at least one micropump coupled to at least one reservoir; wherein the microvalves 216 in the closed state allow fluid to be stored and sealed within the reservoirs and the microvalves 216 in the open state allow fluid to flow between the reservoir and the diagnostic portion 220; and wherein the micropump may be actuated to cause fluid movement from the reservoir to the diagnostic portion such that a plurality of reagents can be preloaded and stored in a sealed manner within the microfluidic cartridge 200 until use. A notch is provided in each of the layers in one corner.

FIG. 12B shows the detailed drawing of the microvalve membrane of the same example embodiment of FIG. 12A. In this example, the microvalve membrane 240 is a film having six microvalves 216g, 216h, 216i, 216j, 216k and 216l, Microvalve membrane 240 may be at least partially made of parafilm and attached to a top part 250 (as shown in FIG. 12C) of the microfluidic cartridge 200 by any adhesive means known in the art. In yet some other embodiments, the microvalve membrane may be made of polyurethane and/or Nylon. Microvalve membrane 240 may be fixedly attached to the top part after the reagents are loaded or supplied through the inlets (not shown) to seal the reagents for storage until use, during the manufacturing process. The film covers and seals the microfluidic portion 210 of the microfluidic cartridge 200. In some embodiments, the microvalve membrane 240 (and the microvalves 216) are made of materials that expands and changes its shape in response to stimuli such as temperature as described in the previous examples. Microvalves 216g, 216h, 216i, 216j, 216k and 216l are flat and seal the corresponding inlet/outlet junctions 218g, 218h, 218i, 218j, 218k and 218l, respectively, for the corresponding reagent reservoirs (as shown in FIG. 12D) in a normally closed state. In this example embodiment, when the microvalves 216g, 216h, 216i, 216j, 216k and 216l are exposed to heat, the materials of the microvalves 216g, 216h, 216i, 216j, 216k and 216l expand and change into a dome shape (i.e. an open state), allowing fluid communication between the inlet/outlet junctions 218. In some example embodiments, the opening of the microvalves is irreversible, that is, the microvalves are single-use and cannot be closed again after opening, avoiding re-using the microfluidic cartridge by a user. In yet another example embodiment, the opening of the microvalves is reversible. In some example embodiments, the microvalve membrane can be transparent or semi-transparent, allowing users to observe the flow of fluids in the microfluidic cartridge 200. In a further example embodiment, the microvalves area can be in dark color such as black for better absorption of light (and thus heat) energy. In this example, the microvalves 216g, 216h, 216i, 216j, 216k and 216l are represented as black spots on the microvalve membrane.

FIG. 12C and FIG. 12D show the detailed structure of the two opposite sides of the top part 250 of the sample example embodiment of FIG. 12A, respectively. In the same example embodiment, the top part 250 includes the microfluidic portion 210 and the diagnostic portion 220 in one corner next to the notch. In some example embodiments, at least one reservoir is filled with at least one fluid, wherein the fluid is a reagent and is sealed with a microvalve. In some example embodiment, at least one reservoir for holding at least one sample further comprises a sample inlet 217 having a removable cap. In some example embodiments, the microfluidic cartridge further comprises a plurality of reagents pre-loaded, sealed and stored separately in a number reservoirs; and at least one reactant pre-supplied at the diagnostic portion. Each reservoir has an inlet and an outlet, which may be connected by the fluid channels. The microfluidic portion 210 includes a sample inlet 217 which is sealable by a sample cap 212 (as shown in FIG. 12A) and a microchannel extended from sample inlet 217 (as shown in FIG. 12D). In this example, microfluidic portion 210 also includes a reservoir 213n for holding introduced sample (or a sample reservoir), six reservoirs 213g, 213h, 213i, 213j, 213k and 213l for storing reagents (or reagent reservoirs), and a reservoir 213m for holding the waste (or waste reservoir). The reservoirs may be in tubular form in any desirable shape, for example, S shape as shown in FIG. 12D. At one end of the sample reservoir 213n, there is a microchannel in fluid communication with the sample inlet 217 (after passing through the microporous membrane) and at the opposite end of the sample reservoir 213n, there is another microchannel connected to other microchannels from other (reagent) reservoirs. Reagent reservoirs 213g, 213h, 213i, 213j, 213k and 213l include inlets 219g, 219h, 219i, 219j, 219k and 219l) at one end respectively, which are capable for introducing at least one reagent to the corresponding reservoirs. At the opposite end of the reagent reservoirs, there are inlet/out junctions 218g, 218h, 218i, 218j, 218k, 218l, which are sealed by the corresponding microvalves 216g, 216h, 216i, 216j, 216k and 216l (as shown in FIG. 12B) correspondingly. Each pair of inlet/outlet junction 218 comprises an outlet from a reagent reservoir and an inlet to a microchannel connecting to the other parts of the microfluidic portion. The microchannels connect the sample reservoir and/or the reagent reservoirs to the diagnostic portion. The reservoirs may be interconnected by the microchannels. In this example, the microfluidic portion 210 also includes a sample reservoir 213n and a removable sample cap 212 is shaped to match the diameter of the inlet of the sample reservoir 213n. The diagnostic portion 220 is in fluid communication with the microfluidic portion 210 through the inlet 226 and outlet 228. In some embodiments, the microfluidic cartridge further comprises at least one reservoir for holding waste. In some embodiments, the microfluidic portion further comprises a waste reservoir 213m, wherein the waste reservoir 213m is connected to the diagnostic portion via an outlet for receiving waste fluid discharged from the diagnostic chamber. FIG. 12D shows the opposite side of the top part of the same example embodiment of FIG. 12C. In this example, the fluid channels are microchannels. In some embodiments, the reservoirs may be configured to form a chamber for holding fluids and can be in any desired shapes and formats. In this example, the reservoirs are designed as S shape to save space such that the microfluidic cartridge could be compact and small in size. In some embodiments, the diagnostic portion comprises a diagnostic chamber for receiving at least one fluid from the microfluidic portion. In some embodiments, the diagnostic portion is at least partially transparent for optical detection.

Now referring back to FIG. 12A of the same example embodiment of the microfluidic cartridge 200, which shows a first adhesive layer 270a. In this example, the first adhesive layer 270a attaches the bottom (opposing) side of the top part 250 to the microporous membrane 260 and the top side of the micropump membrane 280. Any suitable plastic materials such as polycarbonate with a coating of adhesive material on two opposing sides may be used for making the first adhesive layer 270a. In this example embodiment, the first adhesive layer 270a has a thickness and includes seven grooves 271a, which are cavities for accommodating the spaces of the reservoirs for reagents and sample. The thickness of the adhesive layer 270a may correspond to the thickness of the reservoirs in the top part. The position of the grooves 271a corresponds to the respective position of the reservoirs, allowing the reservoirs in direct contact with the next layer (i.e., the micropump membrane 280). In some embodiments, the microfluidic cartridge 200 further comprises a microporous membrane configured to remove gas in the fluids such as a sample and/or one or more reagents. The first adhesive layer 270a also includes a number of microchannel openings 272 (five microchannel openings as shown in FIG. 12A). Microchannel openings 272 serve as the connecting channel for the transportation of fluids between the discontinued microchannels in the top part 250. Microchannel openings 272 forms the fluid channels to connect between the microchannels in top part 250 and the microporous membrane 260, allowing the fluids such as sample, reagents and waste passing the microporous membrane 260, thereby removing any gas bubbles in the fluids. The microporous membrane 260 may be made of any hydrophobic materials which is permeable to gas but impermeable to liquids, for example, PTFE.

FIG. 12A also shows a micropump membrane 280, which is disposed between the first adhesive layer 270a and the second adhesive layer 270b, allowing direct attachment between the top part and the bottom part. The micropump membrane 280 acts as a divider between the reservoirs and the micropumps, avoiding direct contact of the hydrogels in the micropump with the fluids such as sample and reagents. The micropump membrane 280 also includes a groove 283, which is a cavity for accommodating the space of the microporous membrane 260, allowing direct contact of the microporous membrane 260 with the ambient. The micropump membrane 280 can be made of parafilm or plastics such as polyurethane and/or Nylon. Micropump membrane 280 covers all reservoirs to prevent fluid leakage. They work with the micropumps to push the fluids out of the reservoirs by the action of micropump of the cartridge.

FIG. 12A also shows the second adhesive layer 270b. In this example, the second adhesive layer 270b attaches the opposite side of the micropump membrane 280 to the bottom part 290. Any suitable adhesive materials may be used for making the second adhesive layer 270b. In this example embodiment, the second adhesive layer 270b has a thickness and includes seven grooves 271b, which are cavities for accommodating the spaces of the micropumps. The thickness of the adhesive layer 270b may correspond to the thickness of the micropump. In this example, hydrogels (not shown) act as the micropump and disposed within the grooves 271b of the second adhesive layer 270b. The position of the grooves 273 corresponds to the respective position of the reservoirs, allowing the reservoirs in direct contact with the next layer (i.e., the micropump membrane 280) so that the micropumps of the microfluidic cartridge 200 can act on the reservoirs to push the fluids such as sample and reagent out of the reservoirs. Referring now to FIGS. 12E and 12F, which show the two opposing sides of the bottom part 290 of the same example embodiment of the microfluidic cartridge of FIG. 12A. The bottom part 290 may be made of electrical insulated material such as plastic and resin material. The bottom part 290 of the microfluidic cartridge has a groove 291 for a microporous membrane 260 to be placed therein. The bottom part 290 also includes a number of electrical conductive circuit traces 294g, 294h, 294i, 294j, 294k and 294l (FIG. 12E) which are in electrical connection with the electric connecting interface 293 of the opposing side of the bottom part 290. Electric connecting interface 293 transmits electricity from the apparatus to the microfluidic cartridge 200 to activate the micropumps. A micropump (not shown) which is constructed with small amount of hydrogels juxtapose a corresponding reservoir (See FIG. 12D). The hydrogels of the micropumps 215 are in contact with their corresponding electrical conductive circuit traces 294g, 294h, 294i, 294j, 294k and 294l respectively that are incorporated onto the built material of the bottom part 290. These micropumps are operated by electrical current, which are supplied through electrical conductive circuit traces 294. When the electrical current is received, the electrical conductive traces 294g, 294h, 294i, 294j, 294k and 294l transmit electricity to electrolyze the hydrogels to produce gases which push the micropump membrane 280 upward so as to push the fluid out the reservoirs. These micropumps push the sample and reagents through the microchannels whereby the sample is mixed with the reagents to the microchannels by expanding and contracting the hydrogels. The expanding and contracting of the hydrogels are controlled by the cartridge driver unit of the portable diagnostic apparatus 300 by sending signals and power through the connection between the electrical connector of the cartridge receiving unit 310 and the electric connecting interface 293 of the bottom part 290, which is also in electrical connection with the electrical conductive circuit traces 294g, 294h, 294i, 294j, 294k and 294l of the bottom part 290. In some embodiments, the micropumps are encapsulated so that contamination and cross-contamination issues can be avoided. In some embodiments, the pumps are in direct contact with the fluids such as reagents or samples within the reservoirs. In some embodiments, the microfluidic cartridge further comprises a micropump membrane for sealing the hydrogel. In this example, the microfluidic cartridge 200 includes a micropump membrane 280 which covers entire area of the reservoirs to prevent any fluid leakage and direct contact of the hydrogels with the fluids within the reservoirs. The bottom part 290 also includes electricity sensing elements 292 (FIG. 12E) which is juxtapose the area of the waste reservoir for detecting the presence and amount of fluid flowed into the waste reservoir using capacity sensing techniques.

### EXAMPLE 8

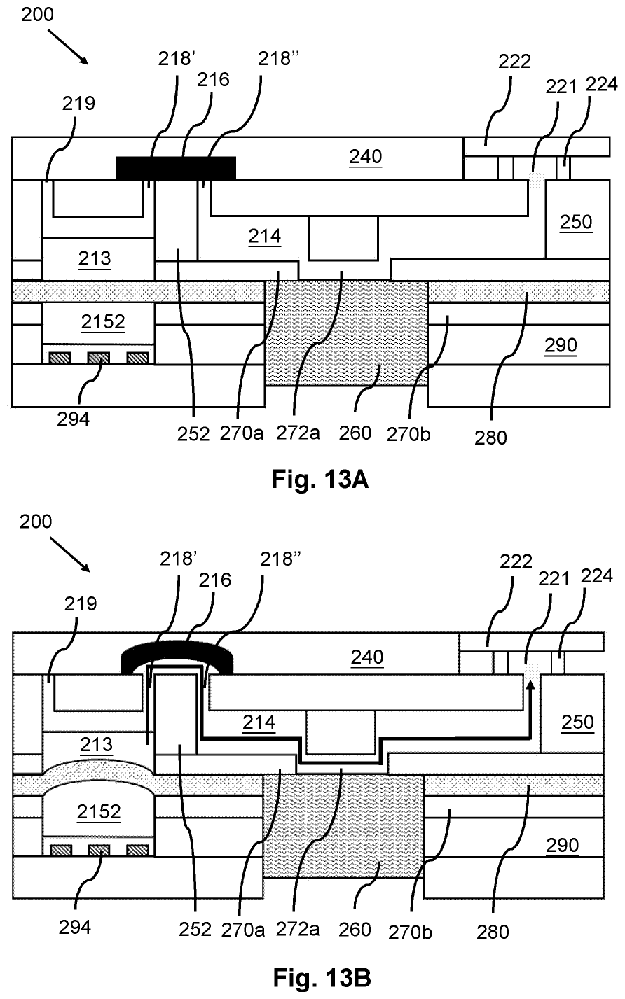

Referring now to FIGS. 13A and 13B, showing how an example microfluidic cartridge 200 operates during use. For clarity and simplicity, only one set of reagent reservoir, microvalve, hydrogel or micropump and inlet/outlet junction are illustrated. During manufacturing process, each reagent is first loaded or supplied to a reagent reservoir 213 through the reagent inlet 219. Then, the surface having reagent inlet 219 of the top part 250 is sealed by a microvalve membrane 240 to prevent fluid leakage from the reagent inlet 219 and outlet 218′ of the reservoir 213. A valve seat 252 may be configured to support the microvalve 216 from collapsing at a resting state. The valve seat 252 is positioned between the reservoir 213 and a fluid microchannel 214 to separate these two compartments such that when the microvalve 216 is in a closed state, the fluid within reservoir 213 is not able to flow through the fluid microchannel 214. Outlet 218′ and inlet 218″ forms the inlet/outlet junction, which is sealed by the microvalve 216. Microvalve 216 on this microvalve membrane 240 is arranged juxtapose each pair of fluid inlet and outlet junction (218′ and 218″) of the top part 250. The reactant such as an antibody may be pre-coated onto the diagnostic chip 222 within the confined area of diagnostic chamber 221 formed together with the adhesive tape 224. Different parts, i.e., microvalve membrane 240, top part 250, adhesive layers 270a, 270b, micropump membrane 280, microporous membrane 260 and bottom part 290 of the microfluidic cartridge 200 are assembled together by adhesive means or welding process. The microfluidic cartridge 200 may be sealed and packed for shipping.

When in use, a fluid containing sample is introduced to the microfluidic cartridge 200 by opening the sample cap and introducing the sample through sample inlet (not shown). Suitable sample preparation may be performed prior to sample introduction. The sample flows into the microchannel and reaches the microchannel opening. At microchannel opening, the sample contacts the microporous membrane for removing any gas bubbles in the fluid. Then, the sample enters the microchannel and flows into the diagnostic chamber 221.

At this point, the sample and reagent reservoirs are filled with all necessary sample and reagents correspondingly. Electrical current from the microvalve controller and the micropump controller is then applied to the microfluidic cartridge 200 to activate the microvalves 216 and the hydrogel 2152 of micropumps of the microfluidic cartridge 200. Referring now to FIG. 13B, a heating element of the microvalve controller (not shown) disposed on the microvalve 216 emits energy such as infrared to the microvalve 216 to open the microvalve 216. This event causes the fluid microchannel 214 to be in fluid communication with reservoir 213 and diagnostic chamber 221. A micropump contains hydrogel 2152, which is in contact directly or indirectly with an electrical conductive trace 294. Electrical conductive trace 294 receives electrical current from the apparatus through electrical connecting interface (not shown) to the hydrogel 2152 at a predetermined sequence. Electric pulses pass through the electrical conductive trace 294 and electrolyze the hydrogel 2152 in that specific reservoir 213. Oxygen and hydrogen are produced from the electrolysis process and these gases expand such that the chamber holding the hydrogel 2152 expands, causing a micropump membrane 280 actively pushes a fluid (sample and/or reagent) inside the reservoir 213 out of the reservoir 213 in a controlled, precise manner. The fluid is pushed out of the reservoir 213 and enters the microchannel 214 through the fluid outlet 218′ and inlet 218″ junction. Arrow in FIG. 13B shows the fluid flow direction. The fluid then reach another microchannel opening 272a. At microchannel opening 272a, the fluid contacts with the microporous membrane 260 to remove any gas bubbles. The fluid then enters to another microchannel and flow into the diagnostic chamber 221, which is a detection area.

At the detection area, the sample reacts with the pre-coated reactant on the diagnostic chip 222. After reaction is completed, the waste flows to the waste reservoir through the waste reservoir inlet (not shown).

The optical inspection is then ready to be performed to the diagnostic portion.

### EXAMPLE 9

### Portable Diagnostic Apparatus

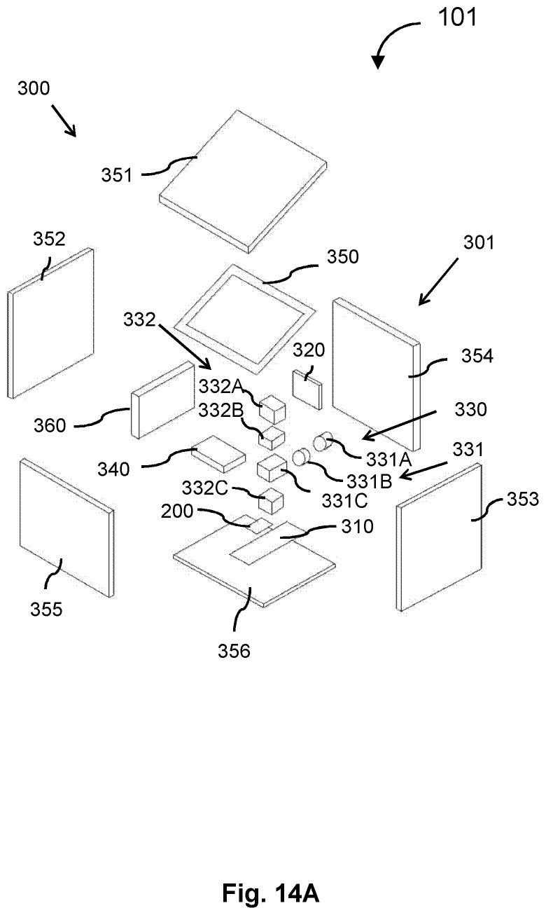

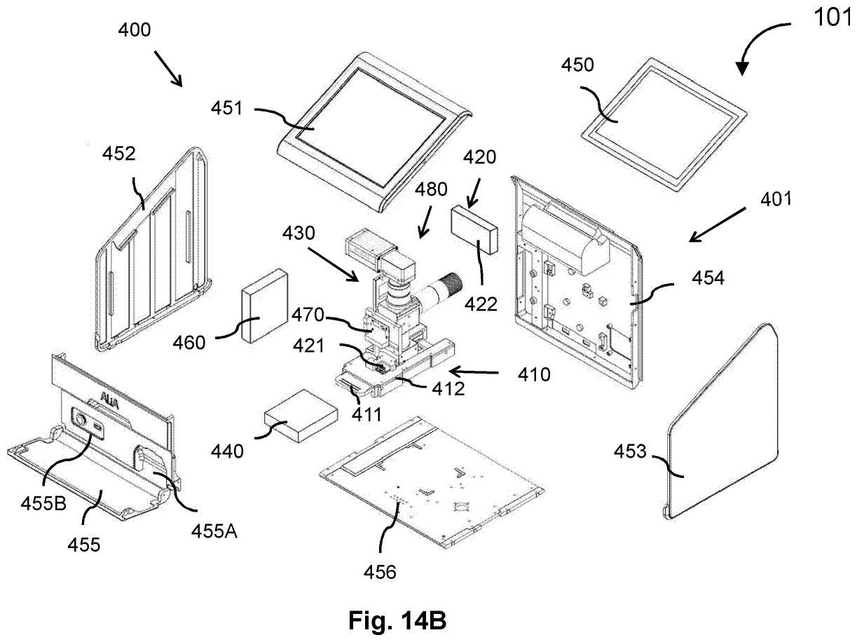

Referring now to FIG. 14A, an example embodiment of a diagnostic system 101 includes a portable diagnostic apparatus 300 and a microfluidic cartridge 200 which operates with the portable diagnostic apparatus 300. In this example, the diagnostic apparatus 300 includes a control unit 340, a microfluidic cartridge driver unit 320, an optical unit 330, a user interface unit 350, a cartridge receiving unit 310 and a power supply unit 360. The portable diagnostic apparatus 300 is enclosed in a housing 301, wherein the housing 301 includes a top cover 351, side covers 352 and 353, a back cover 354, a front panel 355, and a base 356. In some example embodiments, the optical unit 330 comprises an illumination component 331 and a sensor component 332. The illumination component includes a light source 331A, a light tube 331B and a filter 331C. The sensor component includes a camera 332A, camera lens 332B and objective lens 332C.

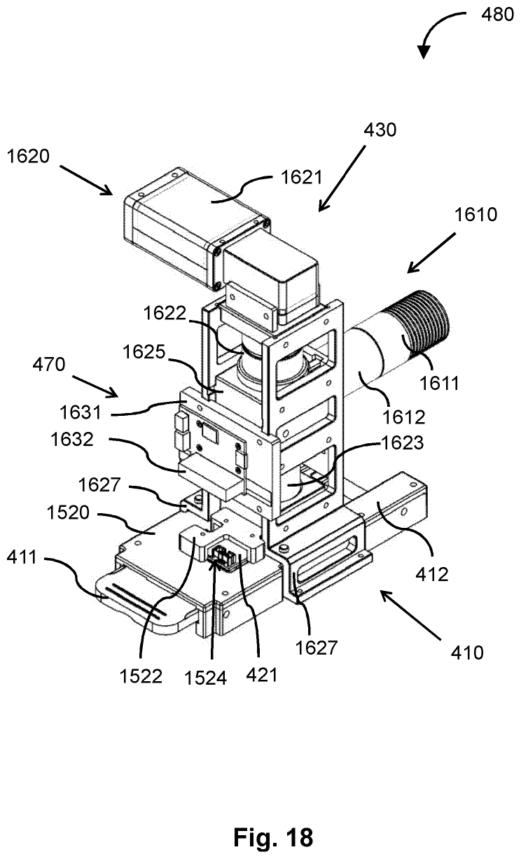

Referring now to FIG. 14B, another example embodiment of the diagnostic system 101 includes a portable diagnostic apparatus 400 and a microfluidic cartridge (not shown), which operates with the portable diagnostic apparatus 400. In some example embodiments, the portable diagnostic apparatus 400 may include all units in the portable diagnostic apparatus 300. In some example embodiments, the portable diagnostic apparatus 400 includes a cartridge driver unit 420, an optical unit 430, a display unit 450, a cartridge receiving unit 410, a power supply unit 460, and an identification unit 470. In one example embodiment, the portable diagnostic apparatus 400 optionally includes a control unit 440. The control unit 440 controls and is operationally connected to the cartridge driver unit 420, the optical unit 430 and the display unit 450. In some other example embodiments, each unit may have a control unit of their own and a single control unit is not present in the portable diagnostic apparatus. In one example embodiment, the microfluidic cartridge driver unit includes a microvalve controller 421 and a micropump controller 422. The microvalve controller 421 and the micropump controller 422 may cooperatively operate to actuate the flow of fluids from the reservoirs to the diagnostic portion in a predetermined sequence when the microfluidic cartridge is placed into the cartridge receiving unit 410. In one example embodiment, the cartridge receiving unit 410 comprises a tray 411 and a rail component 412. In this example, the tray 411 is slidably removable from the cartridge receiving unit 410. The rail component 412 is fixedly attached to the portable diagnostic apparatus 400 and configured to receive the tray 411. In this example, an assembly 480 of the cartridge receiving unit 410, the optical unit 430 and the identification unit 470 are shown in FIG. 14B to illustrate the configuration and spatial relationships between these units. A detailed description of the assembly 480 is disclosed in FIG. 18.

In one example embodiment, the portable diagnostic apparatus 400 is enclosed in a housing 401, wherein the housing 401 includes a top cover 451, side covers 452 and 453, a back cover 454, a front panel 455, and a base 456. In this example embodiment, the rail component 412 of the cartridge receiving unit 410, the microfluidic cartridge driver unit 420, the optical unit 430 and the identification unit 470 are mounted within the housing in a configuration such that there is a space (not shown) for receiving the microfluidic cartridge when the microfluidic cartridge is inserted into the apparatus. The space comprises one or more microvalve locations and one or more micropump locations. The space also includes a reaction location corresponding to the position of one or more microvalves, the one or more micropumps and the reaction site respectively when the microfluidic cartridge is inserted into the space. A more detail description of the space is provided in FIG. 18. In one example embodiment, the front panel 455 of the housing includes a microfluidic cartridge receiving cavity 455A. The microfluidic cartridge is inserted into the space by passing through the receiving cavity 455A.

In some example embodiment, the control unit 440 may have the same configuration as the control unit as described in the previous examples. The control unit 440 controls the quantitative and qualitative analysis, interfacing, and storage of signal obtained from the optical unit 430, and controls and monitors all the operations of the portable diagnostic apparatus 400.

In some example embodiments, the power supply unit 460 may have the same configuration as the power supply unit as described in the previous examples. In some example embodiments, the power supply unit 460 may include a built-in or removable re-chargeable battery.

In one example embodiment, the portable diagnostic apparatus 400 can further include at least one USB port or any other data communication means in a data communication port 455B to allow the operation of common communication protocols of data transfer. In yet another example embodiment, the display unit 450 is equipped in the portable diagnostic apparatus 400 for human interface. The display unit 450 is a high resolution color display that can be either a liquid-crystal display (LCD), Organic Light-Emitting Diode (OLED) or other kind of display. The display unit 450 can be incorporated with a touch screen panel; therefore, it can receive command from the touch of human fingers. The display unit 450 is connected with the control unit 440. However, the way it displays, the content being displayed is made by the graphic user interface.

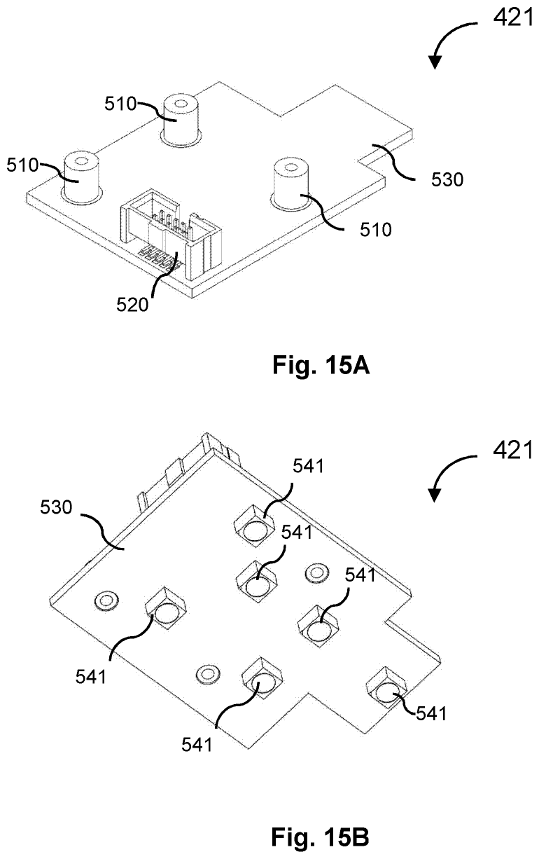

Referring now to FIG. 15A, the microvalve controller 421 of the cartridge driver unit 420 as illustrated in FIG. 14B includes a substrate 530 with a flat surface cut to a shape to fit over a cartridge chamber (not shown in this figure), with a plurality of screw caps 510 and a socket 520 disposed thereon. In this example embodiments, screws (not shown) within the screw caps 510 are used to securely mounted microvalve controller 421 onto the rail component 412 of the cartridge receiving unit 410 in FIG. 14B. The socket 520 is configured to provide electrical connection between the microfluidic cartridge driver unit 420 and the control unit (not shown in this figure) and provide power or electrical current to the microvalve controller 421.

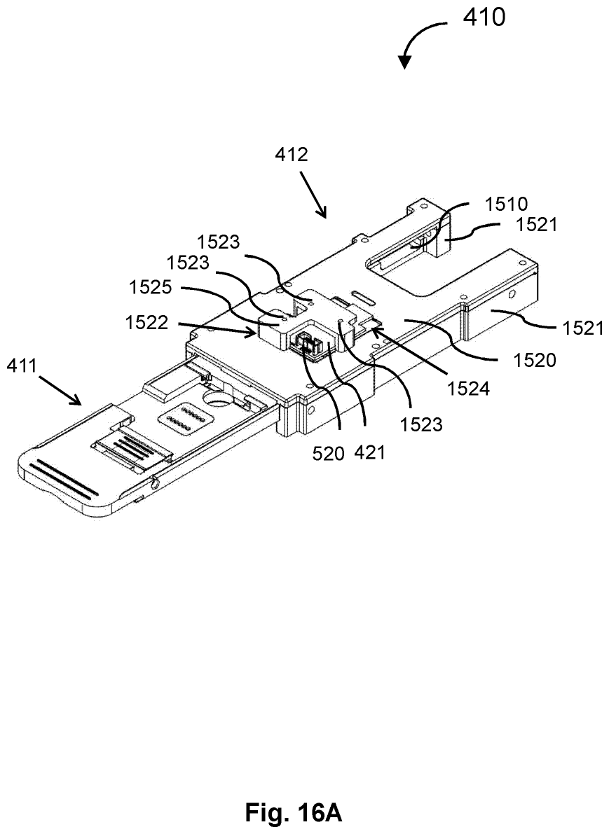

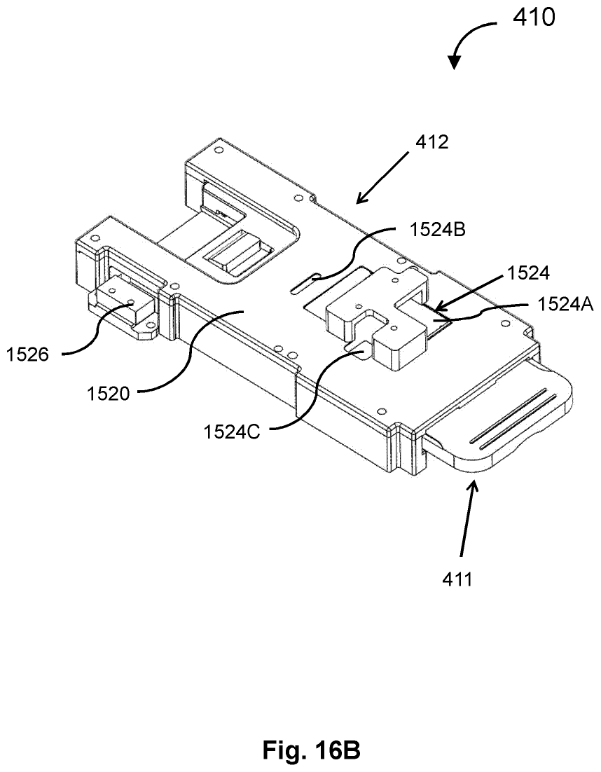

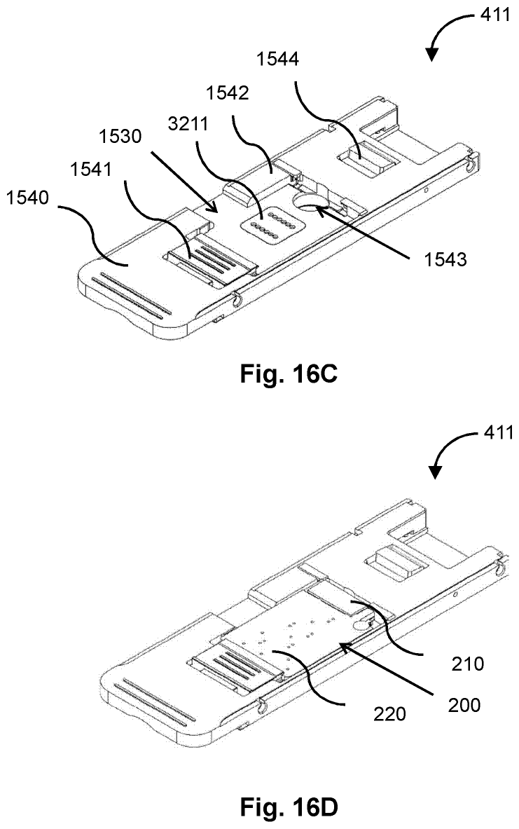

Referring now to FIG. 15B, the microvalve controller 421 includes a number of heating elements 541 (6 in this example) disposed on and extending outwards from the bottom surface of the substrate 530. Heating elements 541 are positioned on the substrate to coincide with the microvalves of a cartridge (not shown in this figure) that the apparatus is designed to work on. When substrate 530 is fixed into position, it is located directly above cartridge chamber (not shown in this figure) and each heating element 541 would be directly positioned above each microvalve when the cartridge is properly inserted into the apparatus and positioned at the designated area. The heating elements 541 are configured to apply heat energy to a heat-deformable surface of the microvalves to cause the microvalves to open. In some embodiments, the heating elements 541 are electromagnetic radiation emitters configured to emit electromagnetic radiation as a source of energy. In some further example embodiments, the heating elements 541 are infra-red (IR) emitters configured to emit IR light as a source of heat energy. Referring now to FIG. 16A, the cartridge receiving unit 410 comprises a tray 411 and a rail component 412. In the example embodiment shown, the rail component 412 includes a flat tray cover 1520 (also referred to as tray board in some embodiments) and 2 sides 1521 defining a partially enclosed compartment. The rail component 412 includes a space with a volume larger than the volume of the tray 411 for slidably receiving the tray such that when the tray 411 with a microfluidic cartridge inserted therein is slided into a docked position, the microfluidic cartridge is positioned at a designated area for reaction and detection. In some embodiments, the rail component 412 is fixedly attached to the portable diagnostic apparatus 400 and configured to receive at least two edges of the tray 411.

In this example embodiment as shown, a pair of slidable rails 1510 is disposed on the inner portion of sides 1521 opposite each other. Only one rail 1510 is shown in FIG. 16A but it is understood that another rail is present on the opposite side of the inner portion of sides 1521. The tray 411 can be anchored on the pair of rails 1510. The pair of rails 1510 slides the tray 411 in and out of the space as aforesaid.

In one example embodiment, the rail component 412 further includes a microvalve controller cover 1522 fixedly attached to the top surface of the tray cover 1520 to hold the microvalve controller 421 in FIGS. 15A and 15B. In some example embodiments, the microvalve controller cover 1522 can be an integral part of the tray cover 1520 rather than a separate part. The microvalve controller cover 1522 provides means for supporting the microvalve controller 421. In this example embodiment, three screws 1523 are used to securely mount the microvalve controller 421 beneath the microvalve controller cover 1522 by fastening the screws 1523 into the screw caps 510 (as shown in FIG. 15A) of the microvalve controller 421. In one example embodiment, the microvalve controller cover 1522 forms an elevated platform 1525 extending upwards from the top surface of the tray cover 1520. The elevated platform 1525 has a shape that includes an opening in the position above the socket 520 of the microvalve controller 421 to allow the electrical wire(s) connected to the socket to pass through. The elevated platform 1525 further includes an opening in the position above a 2D code attached on a microfluidic cartridge (not shown in this figure) when the cartridge is inserted into the space and positioned at the designated area in the apparatus. The opening ensures that the identification unit 470 (as shown in FIG. 14B) positioned above the cartridge receiving unit 410 is able to directly access and scan the 2D code attached on the microfluidic cartridge when the cartridge is positioned at the designated area in the apparatus to identify the cartridge and automatically select the program to run.

Referring now to FIG. 16B, which shows another perspective view of the same cartridge receiving unit 410 in FIG. 16A. In one example embodiment, the tray cover 1520 has an opening 1524 (partially shown) for the microvalve controller 421 to be housed thereon. The size of the opening 1524 is larger or the same as the size of a microfluidic cartridge and divided into three areas: a first area 1524A, a second area 1524B and a third area 1524C. In a further example embodiment as shown in FIG. 16B, the second area 1524B can be a separate opening. The microvalve controller is positioned right next to the first area 1524A such that when a microfluidic cartridge is inserted into the tray 411 and the tray 411 is slided into a docked position, the microvalve on the microfluidic cartridge is positioned directly underneath the microvalve controller 421. As described in FIG. 15B, each heating element 541 of the microvalve controller 421 is positioned directly above each corresponding microvalve of the microfluidic cartridge so that heat can be directed to the microvalve when the cartridge is inserted into the apparatus and positioned at the designated portion. An optical unit (not shown in this figure) is positioned right above the second area 1524B such that the diagnosis portion of the microfluidic cartridge is positioned directly underneath the light source and the camera of the optical unit when the microfluidic cartridge is placed at the designated area.

In some example embodiments, the cartridge receiving unit 410 further includes a switch 1526. In a further example embodiment, the switch is a microswitch. The microswitch can be attached to the back of the rail component 412 and is electrically connected to the power supply unit (not shown). The microswitch is automatically activated when the tray 411 with the microfluidic cartridge inserted therein is slided into the docked position through the rail component 412. Upon activation, the microswitch can switch on the identification unit (not shown) to read the identity of the microfluidic cartridge and automatically choose the program to use.

FIG. 16C shows a schematic view of the tray 411 of the cartridge receiving unit 410 in FIG. 16A. FIG. 16D shows a schematic view of the same tray 411 but with the microfluidic cartridge 200 in FIG. 11A inserted therein. The tray 411 includes a tray plate 1540 and a cartridge chamber 1530. In this embodiment, the cartridge chamber 1530 is configured in the shape of a rectangular block to receive the microfluidic cartridge 200 such that the microfluidic cartridge 200 is positioned at a designated area. The rectangular block shape defines a planar surface and an axis that runs somewhat perpendicular to the planar surface. In one embodiment, the microfluidic cartridge 200 and the cartridge chamber 1530 are configured such that there is only one possible way that the microfluidic cartridge 200 can securely fit into the cartridge chamber 1530 and the tray 411 and thus eliminates chances of inserting the microfluidic cartridge 200 in undesired orientation or position.

Tray 411 serves as the same location or position for (1) the reaction to be run in a predetermined sequence and (2) optical analysis of the microfluidic cartridge. In one example embodiment, the tray 411 includes electrical connectors 3211 disposed on the cartridge chamber 1530 of the tray. The electrical connectors 3211 is configured to receive control signals and power from the micropump controller 422 as shown in FIG. 14B to perform the predetermined sequence by providing electrical current to the micropumps of the microfluidic cartridge 200. In one example embodiment, the electrical connectors 3211 are situated underneath the microfluidic cartridge 200 when the cartridge is inserted in the chamber 1530. The electrical connectors 3211 are in electrical connection with the electrical connecting interface of the cartridge 200 and act as an interface for the cartridge driver unit 420 to drive and control the micropumps in the cartridge 200. In one further example embodiment, the tray 411 includes an opening 1543 on the cartridge chamber of the tray 411. The opening 1543 is configured such that it is situated directly underneath the diagnostic portion 210 of the cartridge 200 when the cartridge is inserted and secured in the chamber 1530 to allow light to pass through the diagnostic portion 210 when optical analysis is performed. The one-tray system eliminates the possibility of human error by design.

In one example embodiment, the tray 411 further includes an anchoring system to ensure that the microfluidic cartridge 200 is secured in the tray 411 when it is inserted into the tray. In one example embodiment, the anchoring system consists of at least one cartridge clip 1541 disposed on the tray plate 1540. In another example embodiment, the anchoring system consists of two cartridge clips 1541 and 1542, positioned orthogonal to each other. The two clips work together to limit the movement of the microfluidic cartridge 200 once it is inserted into the tray 411. Less movement means less variation in the possible position of the bioassay and hence increase in detection accuracy and precision. The addition of extra cartridge clip lowers the tolerance to 0.1 mm and allows more accurate detection as the variation in bioassay position is minimized. In one example embodiment, the tray 411 further includes at least one tray clip 1544 disposed on the tray plate 1540 to secure the position of the tray when the tray slides in the cavity of the rail component 412 (as shown in FIG. 16A).

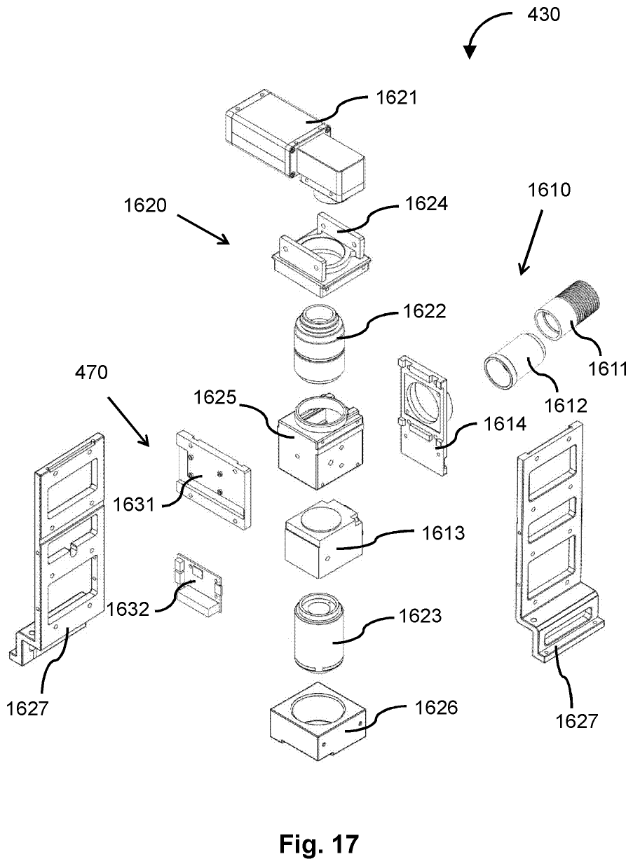

Referring now to FIG. 17, the optical unit 430 comprises an illumination component 1610 and a sensor component 1620. In some embodiments, the illumination component 1610 includes at least one light source 1611 and a light tube 1612. In some example embodiments, the optical unit 430 further includes one or more filters 1613. In some example embodiments, the filter(s) 1613 are contained in a filter cube. In one example embodiment, the sensor component 1620 includes a camera 1621 positioned at the top of the optical unit 430, a camera lens 1622 positioned beneath the camera 1621, and at least one objective lens 1623. In some example embodiments, the optical unit 430 further includes a camera mount 1624 positioned in between the camera 1621 and the camera lens 1622 to connect these two components. In some example embodiments, the optical unit 430 further includes a filter mount 1625 configured to connect the camera lens 1622, the filter(s) 1613 and the objective lens 1623. In one example embodiment, the filter mount 1625 is shaped to include a cavity to receive the filter(s) 1613 such that the filter(s) 1613 can be securely fitted within the filter mount 1625. The filter mount 1625 further includes an opening at the top to connect the camera lens 1622 and the filter(s) 1613, an opening at the side to connect the light tube 1612 and the filter(s) 1613, and an opening at the bottom to connect the objective lens 1623 and the filter(s) 1613. In some example embodiments, the optical unit 430 further includes a cube front 1614 configured to connect the illumination component 1610 and the filter mount 1625 such that the light from the light source 1611 can enter the filter(s) 1613 in a fixed angle. In some example embodiments, the optical unit 430 further includes an objective cover 1626 configured to receive and secure the objective lens 1623. In some example embodiments, the apparatus further includes one or more stands 1627 serving as a scaffold to support the structure of the optical unit 430 when all components are assembled together. The stands 1627 also serves as a scaffold such that the optical unit 430 and the cartridge receiving unit 410 (as shown in FIG. 16A) are securely mounted on the stands in a configuration that are further described in FIG. 18.

When the tray 411 with the microfluidic cartridge 200 inserted therein as shown in FIG. 16D (but not shown in this figure) are at its docked position in the apparatus, the diagnostic portion 210 of the microfluidic cartridge 200 is situated directly below the optical unit 430 for inspection. The illumination component 1610 and the sensor component 1620 are mounted to point towards the diagnostic portion of the microfluidic cartridge. A further description of the arrangement is provided in FIG. 18. The illumination component 1610 is configured to deliver light to the diagnostic portion 210 of the microfluidic cartridge, and the sensor component 1620 is configured to detect at least one signal generated from the diagnostic portion cause by the presence of an analyte when a microfluidic cartridge is inserted and operated at a predetermined condition. The optical unit 430 can be used for on-site analyte analysis/detection. In some embodiments, the apparatus may include one or more optical units 430. The optical unit 430 may acquire images or signals of a sample. The optical unit may send the acquired signals to the control unit to translate the acquired signals into meaningful values.

In some example embodiments, the light source 1611 can be a laser or LED, either monochromatic or polychromatic. This light source 1611 should be strong enough to excite fluorophores. In one example embodiment, the light source 1611 is a LED. In some example embodiments, the light source 1611 can be a high luminosity LED spot light with a blue or red LED color. In some example embodiments, using a LED spot light with a red LED color is advantageous, such as to reduce the autofluorescence of the microfluidic cartridge (not shown), versus using blue, green or other colors. The red light from the light source 1611 may be collimated with a lens and/or a filter 1613 to filter the appropriate wavelength, reflected by a mirror and focused onto the diagnostic portion of the microfluidic cartridge, and imaged with a detector, such as a CCD camera. The red excitation light may excite red-excited fluorophores present in the reacted sample on the diagnostic portion. In some example embodiments, other red-excited fluorophores may be used.

In another example embodiment, the illumination component 1610 comprises a light source 1611 such as a diode laser radiating at least one laser beam with at least one predetermined wavelength on the microfluidic cartridge 200 to generate at least one signal. The predetermined wavelength of the laser beam is selected such that at least one signal which is detectable by the sensor component 1620 can be generated. In one example embodiment, the intensity and the wavelength of the laser beam can be selected/controlled by the user through a control unit (not shown in this figure) for detecting a particular analyte. The laser beam is steered to the microfluidic cartridge at an angle so as to avoid reflections and to generate the signal at higher quality. The predetermined wavelength, for example, is in a range of 465 to 500 nm, 400 to 700 nm, 430 to 465 nm, 500 to 550 nm, 550 to 580 nm, 580 to 620 nm, or 620 to 700 nm.

In one example embodiment, the light source 1611 comprises a light tube 1612 that evenly release light. The light tube 1612 is configured such that it directs the light to other optical components and helps to focus the beam of light onto the bioassay on the diagnostic portion of the microfluidic cartridge when the cartridge is positioned at the designated area. The light tube 1612 is aligned with the bioassay on the diagnostic portion which is located at a specific position on the microfluidic cartridge when the cartridge is positioned at the designated area for reaction and analysis. In one example embodiment, the illumination component 430 may further include at least one light-focusing lens (not shown) as described in example 1 such that the focusing of the light from the light source 1611 is optimized.

In one embodiment, the optical unit 430 may include one or more light sources, one or more lenses, one or more dichroic mirrors, one or more sensors, one or more emission filters and/or one or more excitation filters.

When the tray 411 with the microfluidic cartridge 200 inserted therein as shown in FIG. 16D (but not shown in this figure) are at its docked position in the apparatus, the microfluidic cartridge 200 is located beneath the sensor component 1620 and the illumination component 1610. The sensor component 1620 receives signals from the diagnostic portion of the microfluidic cartridge generated by radiating a light beam on the diagnostic portion by the illumination component 1610. The received signals are then sent to the control unit (not shown) for analysis.

The sensor component 1620 can be of a high quantum efficiency in the wavelength range that it is detecting in. In one example embodiment, the camera 1621 of the sensor component 430 can be a charge-coupled device (CCD) or any other suitable camera. In one example embodiment, the camera 1621 is a near-infrared optimized camera with a type 2/3 (11.0 mm diagonal) CCD sensor.

The camera lens 1622 of the sensor component 1620 can be any suitable lens for camera 1621 or a lens of a higher quality such as microscope-grade lens, depending on the type of immunoassay used. In one example embodiment, the camera lens 1622 is responsible for assisting the camera 1621 to focus on the bioassay since the bioassay is physically small. In one example embodiment, the camera lens 1622 is a C-mount lens. In some embodiments, the C-mount lens is located between the camera 1621 and the objective lens 1623. In some example embodiments, the C-mount lens is securely attached to the camera 1621. In one example embodiment, the C-mount lens has an effective focal length of 20-30 mm.

In one example embodiment, the optical unit 430 includes one or multiple filter(s) 1613. The filter(s) 1613 can be used to filter out any light produced from the light source that is of unwanted wavelength and any undesirable noise in the signals that the camera picks up.

One or more than one filters 1613 can be used depending on the light source 1611 and the fluorophore used. The illumination component 1610 is connected to the filter 1613. In one example embodiment, the camera 1621 is connected to the camera lens 1622 and these two components are connected to the filter(s) 1613. The filter(s) 1613 is mounted and aligned between the camera lens 1622 and the objective lens 1623. This connection to the filter(s) 1613 allows any undesirable signal such as noise, which is often of a different wavelength, either produced by the bioassay or by any undesirable reaction, to be filtered and hence minimize unwanted interference with real signals. In one example embodiment, the filter(s) 1613 is also connected to a light tube 1612 at a certain angle which helps to focus the filtered light beam onto the bioassay. In a further example embodiment, the angle is 0 degree. In yet another example embodiment, the angle is between 1 to 50 degrees. In one example embodiment, the filter(s) 1613 is a filter set containing one or more dichroic filters, one or more emission filters, and one or more excitation filters. In one example embodiment, the filter set is configured to alter the light path such that the light generated by the light source 1611 can be directed to illuminate the diagnostic portion of the microfluidic cartridge perpendicularly to the axis of the cartridge chamber when the microfluidic cartridge is positioned at the designated area.

In one example embodiment, a fluorescence filter set for CY5 Fluorescein is used. In a further example embodiment, the fluorescence filter set for CY5 Fluorescein has a specification shown as follows:

Excitation Band (nm): 600-650

Emission Band (nm): 670-710

Dichroic Reflection Band (nm): 550-650

Trans Band (nm): 650-800

In some example embodiments, the optical unit 430 provides a filtered light beam with a wavelength in the range of 400 nm to 700 nm. In a further example embodiment, the optical unit 430 provides a filtered light beam with a wavelength in the range of 600 nm to 650 nm.

In one example embodiment, the objective lens 1623 of the sensor component 1620 is a plan achromat, 4× magnification with an effective focal length of 45-55 nm and a coating covering wavelengths from UV to NIR.

FIG. 17 also shows an identification unit 470 of the portable diagnosis apparatus 400 as illustrated in FIG. 14B. The identification unit 470 is configured to read the identity of the microfluidic cartridge (not shown in this figure) and transmit a corresponding identity signal to a control unit (not shown). In some example embodiments, the identification unit 470 comprises a reader 1632 configured to read the identity of the microfluidic cartridge. In some example embodiments, the identification unit 470 is attached or fixed onto to the optical unit 430. In one example embodiment as shown in FIG. 17, the identification unit 470 further includes a reader mount 1631 to receive the reader 1632 and attach the reader onto the scaffold of the optical unit 430. The reader 1632 is located right above the designated area where the microfluidic cartridge will be placed and pointing towards the microfluidic cartridge.

In one example embodiment, the reader 1632 is a barcode reader. The barcode reader can read the 2D barcode attached or fixed on the microfluidic cartridge. In one example embodiment, a barcode is attached or fixed onto the microfluidic cartridge (see FIG. 11A).

In a further example embodiment, a two-dimension (2D) code is attached or fixed onto the microfluidic cartridge. A 2D code that incorporates the identity of the microfluidic cartridge, the analytes or diseases to be tested, expiry date of the chip is placed on the microfluidic cartridge during manufacturing process. Upon the insertion of the microfluidic cartridge into the tray, the barcode reader is activated and a 2D code is scanned automatically by the barcode reader. A software can automatically choose the correct program to use based on the 2D code. This feature eliminates the need to manually select the pulse program thus makes the design more user-friendly and less prone to human error. In one example embodiment, the software can also identify a microfluidic cartridge which has been previously used or which is defective. The software shows a warning message on the screen and will not proceed with the program.

Referring now to FIG. 18, the components in the optical unit 430, the identification unit 470, the microvalve controller 421 of the cartridge driving unit 420 and the cartridge receiving unit 410 of the portable diagnostic apparatus 400 as illustrated in FIG. 14B are arranged such that a compact integrated assembly 480 is formed. This compact design leads to a smaller, and lighter diagnostic system. The diagnostic system should be small and light enough to be moved from clinic to clinic if needed. In one example embodiment, the present invention is small and light enough to be hand-carried onto a domestic aircraft. In one example embodiment, the dimension of the apparatus is approximately 30×30×30 cm³ and the weight is approximately 5-6 kg.

FIG. 18 illustrates the configuration and spatial relationships between the units integrated together in the assembly 480. The spatial arrangement of the components in each unit is described earlier. In one example embodiment, the camera 1621 and the camera lens 1622 mounted thereon are positioned on one side of the rail component next to second area 1524B of opening 1524 (not shown in this figure but shown in FIG. 16A) and aligned axially to the plane of the cartridge chamber. In this figure, it is show at the top of the assembly 480. These two components are connected to the filter(s) (not shown) fitted in the filter mount 1625. The filter is mounted and optically aligned between the camera lens 1622 and the objective lens 1623 positioned beneath the filter mount 1625. A light tube 1612 of the illumination component 1610 is also attached to the opening at the side of the filter mount 1625 and optically aligned to illuminate perpendicularly to the axis of the cartridge chamber on the same side of the rail component 412 as the camera lenses. When a light beam generated by the light source 1611 enters the filter(s), the filter(s) allows only light of a specific wavelength or range of wavelength to pass through and reach the bioassay and filters out light of unwanted wavelengths. In this embodiment, the rail component 412 of cartridge receiving unit 410 is positioned underneath the objective lens 1623 of the optical unit 430. In one embodiment, the optical unit 430 and the rail component 412 are securely mounted on two stands 1627 positioned at opposite sides of the assembly 480 such that they are held together as an integral part in the apparatus. Each stand 1627 has a length that extends from the bottom of the rail component 412 to the camera 1621 and is shaped to fit over the optical unit 430 and the cartridge receiving unit 410 to support the structure of the assembly 480 when all components are assembled together. In one example embodiment, the reader mount 1631 of the identification unit 470 with the reader 1632 mounted thereon is attached onto the scaffold of the optical unit 430 by mounting on the two stands 1627 and aligned with the third area 1524C of the opening 1524. In this embodiment, the reader 1632 is located above the cartridge receiving unit 410 and point towards the designated area where the microfluidic cartridge (not shown) will be placed to scan the code attached on the microfluidic cartridge.

In this example embodiment as shown, the rail component 412 of the cartridge receiving unit 410, the microfluidic cartridge driver unit 420, the optical unit 430 and the identification unit 470 are mounted within the housing in a configuration such that there is a space (not shown) for receiving the microfluidic cartridge when the microfluidic cartridge is inserted into the apparatus. In this example embodiment, when the microfluidic cartridge is inserted into the tray 411 and the tray 411 is slided into a docked position, the microfluidic cartridge is positioned in the designated area of the space. The space comprises one or more microvalve locations and one or more micropump locations.

The microvalve controller 421 is securely mounted on the rail component 412 as shown and is positioned juxtapose to the microvalve of the microfluidic cartridge inserted in the space such that the heating elements of the microvalve controller 421 corresponds to the position of the microvalves. The illumination component 1610 and the sensor component 1620 are aligned axially to the plane of the cartridge chamber and mounted to point directly towards the diagnostic portion of the microfluidic cartridge. The openings 1524 ensures that the diagnostic portion of the microfluidic cartridge is situated directly underneath the camera lens of 1623 the optical unit 430 for inspection. The sensor component 1620 receives signals from the diagnostic portion of the microfluidic cartridge generated by radiating a light beam on the diagnostic portion by the illumination component 1610. In one embodiment, the received signals may be sent to the control unit (not shown) for analysis.

In some example embodiments, the assembly 480 further includes a switch 1526 (not shown in this figure but shown in FIG. 16B). In a further example embodiment, the switch 1526 is a microswitch. The microswitch can be attached to the back of the rail component 412 and is electrically connected to the power supply unit (not shown). The microswitch is automatically activated when the tray 411 with the microfluidic cartridge inserted therein is slided into the docked position through the rail component 412. Upon activation, the microswitch can switch on the reader 1632 to read the identity of the microfluidic cartridge 200 and automatically choose the program to use.

The events associated with the assembly 480 of the apparatus are described below:
S1. The user puts the microfluidic cartridge into the cartridge chamber of the tray 411.
S2. The user pushes the tray 411 into the apparatus as guided by the rail component 412.
S3. Switch 1526 is activated as the tray 411 is pushed in and secured in the docked position.
S4. The switch 1526 switches on the barcode reader 1632.
S5. The reader 1632 reads a code that is printed or attached onto the microfluidic cartridge.
S6. The code, which contains the identity of the microfluidic cartridge, prompts the software of the control unit to automatically select the program that is associated with this microfluidic cartridge.
S7. Once the software has selected the correct program, the microvalve controller 421 and the micropump controller (not shown in this figure) cooperatively operate to actuate the flow of fluids from the reservoirs to the diagnostic portion in a predetermined sequence.
S8. Once the reaction is completed in the diagnostic portion, the illumination component 1610 is activated and the bioassay is excited by a light beam generated by the light source 1611.
S9. The sensor component 1620 captures an optical image and the image is analyzed by the software.
S10. The result is shown on the screen for the user to see. No human interpretation is required.

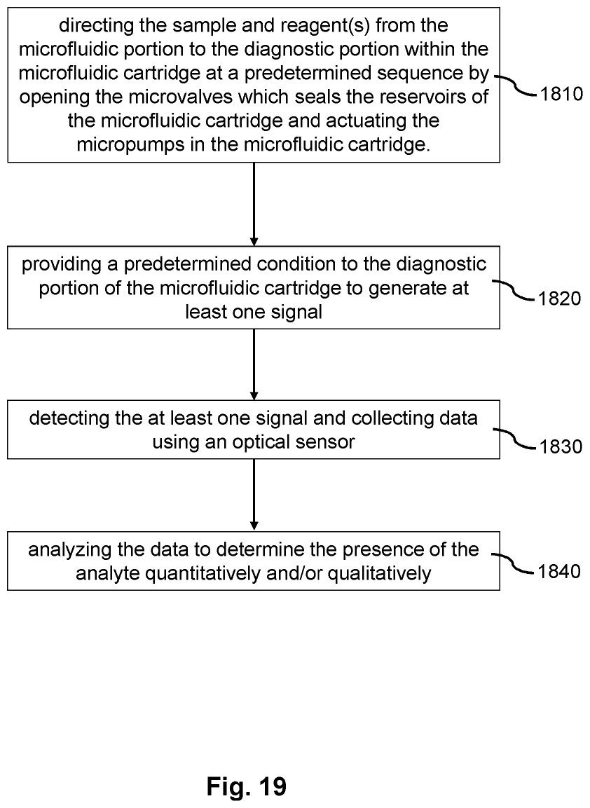

In one exemplary embodiment, the operation of the diagnostic apparatus when a microfluidic cartridge are inserted therein are shown in FIG. 19 and are detailed below:

Block 1810 states directing the sample and reagent(s) from the microfluidic portion to the diagnostic portion within the microfluidic cartridge at a predetermined sequence by opening the microvalves which seals the reservoirs of the microfluidic cartridge and actuating the micropumps in the microfluidic cartridge.

In some example embodiments, the predetermined sequence includes a spreading analyte step. In the spreading analyte step, the fluids such as sample, buffer(s) and reagent(s) exit the channel opening in sequential order, spreading across the diagnostic chamber and the fluids are in direct contact with the diagnostic chip. The area where the sample, buffer(s) and reagent(s) spread covers the place where the array of detection spots locates such that the analyte(s) can react/interact with the analyte interacting molecule pre-coated on the detection spots. In one example embodiment, the spreading analyte step can further includes the step of further driving the microfluidic cartridge to spread a second auxiliary reagent, which is located at one of the reservoirs, by flowing through the microchannels to the diagnostic portion for attaching a secondary molecule for facilitating the detection of reacted or interacted analyte after the sample and reagent are spread on the array of detection spots. In yet another example embodiment, the pre-coated analyte has bound to a molecule for the detection of an analyte without the need for a secondary molecule. The molecule can be a molecule that can generate a fluorescent signal or other detection signals for subsequent analyzing step.

In one example embodiment, the micropump controller of the cartridge driving unit generate a specific sequence of electrical pulses and the electrical pulse sequence passes through the microfluidic cartridge via the electrical connectors that are located at the bottom of the tray. This sequence of electrical pulses can drive the micropump to facilitate the fluid movement within the microfluidic cartridge. At the same time, the microvalve controller received signals from the cartridge driver unit to apply heat energy to a particular microvalve location on the microfluidic cartridge, causing the microvalve to open. The microvalve controller and the micropump controller operate cooperatively to drive the sample or reagents in the microfluidic cartridge out of their reservoirs and push them into the diagnostic portion in a pre-determined sequence.

In one example embodiment, the control unit of the apparatus includes a microfluidic cartridge driver software module to control the fluid actuation. The microfluidic cartridge driver software module is designed to instruct the cartridge driver unit to control the electrical current and the time of delivering such electrical current to the micropump of the microfluidic cartridge. The higher the electrical current and/or the longer the time for delivering such electrical current, the more fluids can then be pumped from the reservoirs.

The microfluidic cartridge driver software module is also designed to instruct the cartridge driver unit to control the electrical current and the time of delivering such electrical current to the microvalve controller, which controls the opening of the microvalve by emitting heat energy onto the surface of the microvalve, causing the microvalve to expand.

Block 1820 states providing a predetermined condition to the diagnostic portion of the microfluidic cartridge to generate at least one signal.

In one example embodiment, the predetermined condition includes an analyzing step. The diagnostic portion of the microfluidic cartridge is located underneath the optical sensor and does not separate from the microfluidic cartridge after the spreading analyte step. Upon the receiving of the starting signal from the control unit, light beam from the illumination component (e.g. a laser beam) of the optical unit is filtered and directed onto the diagnostic portion to generate at least one signal (if the sample contains the analyte) detectable by the sensor component. In one embodiment, the at least one signal includes fluorescence signal which is generated when the diagnostic portion is radiated by the suitable light at suitable wavelength

Block 1830 states detecting the at least one signal and collecting data using an optical sensor.

In one example embodiment, the signal (fluorescent signal for example) as aforesaid are filtered by the filter(s) of the optical unit and collected by the sensor component of the optical unit positioned above the diagnostic portion. The filter(s) is used to filter out any undesirable noise in the signals that the camera of the sensor component picks up.

Block 1840 states analyzing the data to determine the presence of the analyte quantitatively and/or qualitatively.

In one example embodiment, the signal collected will be converted into digital data which will then be transferred to and analyzed by the microprocessor of the control unit to determine the presence of the analyte quantitatively or qualitatively. In one example embodiment, the result will be shown on the display unit of the apparatus in relatively short period of time. The entire process time (i.e., from inserting the microfluidic cartridge into the apparatus to showing the results) will only takes 10-25 mins. In yet another example embodiment, the entire process time takes only around 15 mins.

In one example embodiment, the present invention has been designed to have minimal human involvement which minimizes the chances for human error. With all reagents preloaded into the microfluidic cartridge and sealed, only the sample chamber inlet is exposed and is the only obvious inlet for where the sample should be loaded. This design minimizes the chance for the user to load the sample into a wrong chamber. As the microfluidic cartridge is inserted into the apparatus, the barcode reader scans the data matrix on the microfluidic cartridge and either rejects the cartridge if it has already been used or accepts the microfluidic cartridge and automatically selects the correct test program. This feature prevents any used microfluidic cartridge to be accidentally re-used and prevents the user from making mistakes when selecting the test program on the diagnostic platform. The software embedded in the diagnostic platforms analyzes and shows the test results on the screen which eliminate any chances of human misinterpretation when reading the results.

### EXAMPLE 10

Apparatus with Smart Capabilities

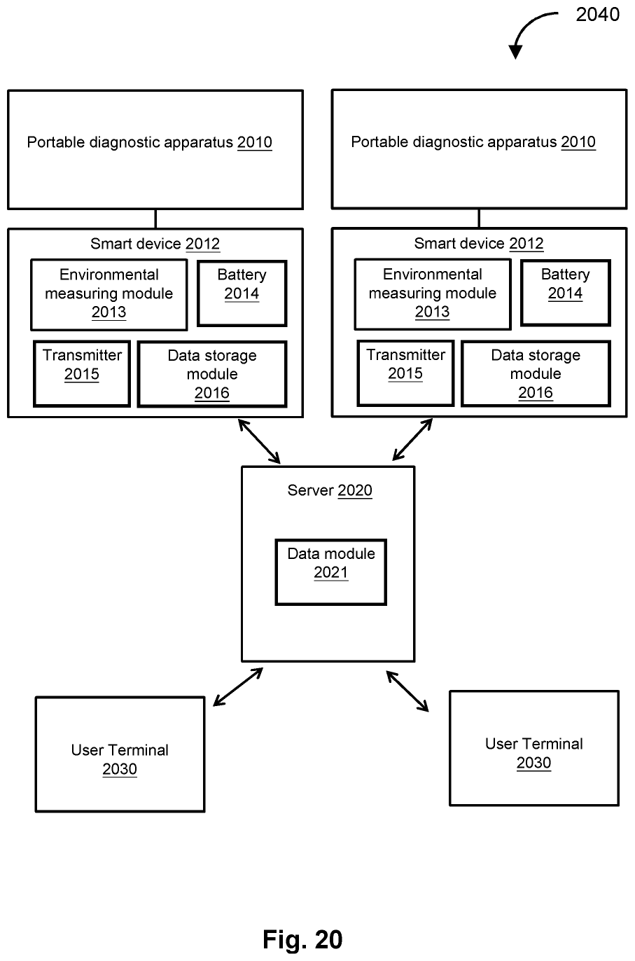

Referring now to FIG. 20, one aspect provides a portable diagnostic apparatus 2010 further includes a removable smart device 2012 optionally connects and communicates with the portable diagnostic apparatus 2010 for obtaining disease prevalence information in a location (as shown in FIG. 10B), wherein the smart device 2012 comprises

an environmental measuring module 2013 for acquiring environmental data, wherein environmental data comprises at least one environmental parameter at the location;

a data storage module 2016 for storing raw data, wherein the raw data comprises one or more of environmental data and diagnostic data;

a transmitter 2015 for transmitting the raw data to a remote server; and

a battery 2014.

The portable diagnostic apparatus 2010 may be used to collect different types of diagnostic data. The diagnostic data may include disease type, disease severity, viral load, presence or absence of pathogen or allergens, or blood count. Examples of diagnostic data include, but are not limited to data associated with (1) animal diseases such as Porcine Reproductive and Respiratory Syndrome (PRRS), Bovine Foot-and-Mouth Disease (FMD), Classical Swine Fever (CSFV) infection, and Bovine Spongiform Encephalopathy (BSE) Infectious Disease) (2) food safety (e.g. detection of food allergens (e.g. peanuts, seafood), aflatoxin and melamine) and (3) human diseases such as infectious diseases (e.g. sexually transmitted diseases (STD), Middle East respiratory syndrome coronavirus (MERS-CoV) and Influenza virus infection), tropical diseases (e.g. Dengue virus and Japanese Encephalitis virus infection) and new emergent infectious diseases which fall within antigen/antibody immunological mechanism in their pathological pathway, Flu A, flu B, RSV, HPIV, adenovirus, dengue, chikungunya, Zika, malaria, leptospirosis, toxoplasmosis, canine distemper virus Ab, canine parvovirus Ab, or heartworm.

In yet other embodiments, the portable diagnostic apparatus 2010 can measure apparatus data, wherein apparatus data is machine information or operation status. In some embodiments, machine information is selected from the group consisting of model, machine identity, machine, hardware version, software version, country originally purchased, and owner. In other embodiments, the operation status is selected from the group consisting of error code, system voltage, total operation hours, and total number of tests.

According to another embodiment, the smart device 2012 comprises an environmental measuring module 2013 for acquiring environmental data, wherein environmental data comprises at least one environmental parameter at the location. In some embodiments, the environmental data is selected from positioning data, humidity, temperature, barometric pressure, time, and air quality (AQI, pollen count, etc). In some embodiments, the positioning data is global position and is acquired by global positioning satellite (GPS). In some embodiments, the environmental data is selected from positioning data, humidity, temperature, and time.

In some embodiments, the environmental measuring module 2013, the transmitter 2015, and the data storage module 2016 together form a smart device 2012 which can optionally connect to and communicate with the portable diagnostic apparatus 2010 and a remote server 2020. In some embodiments, the smart device 2012 is removable. The smart device 2012 can be any size, but in certain embodiments it is smaller than the apparatus and can fit inside the apparatus. In some embodiments, the removable smart device can be put into a casing.

According to other embodiments, the smart device 2012 further comprises a battery 2014. In some embodiments, the battery is rechargeable and can operate 30 days without being recharged. In yet other embodiments, the transmitter 2015 is a wireless transmitter.

Still referring to FIG. 20, another aspect of the invention provides a system 2040 for managing a network of portable diagnostic apparatuses 2010 each connected and communicated with a removable smart device 2012 and obtaining disease prevalence information, comprising at least one user terminal 2030, and a server 2020 comprising a data module 2021 for collecting and storing raw data, wherein the raw data comprises one or more of the following:

diagnostic data obtained at a location using a portable diagnostic apparatus 2010, wherein the diagnostic data comprises at least one biochemical or pathological measurement of a subject,

environmental data,

apparatus data obtained from the portable diagnostic apparatus 2010, and

a data module for analyzing the raw data

wherein the server 2020 is connected to the user terminal 2030 and to the removable smart device 2012.

In some embodiments, the environmental data is obtained at the location using an environmental measuring module 2013 wherein the environmental data comprises at least one environmental parameter. In other embodiments, the environmental data is obtained from a third source, such as from an environmental measuring device or from public records about the environment at the location, such as local news sources, weather observatory reports, or the internet. Examples of environmental measuring devices include, but are not limited to, devices for measuring one or more of humidity, temperature, air velocity, air pressure, light, dust, sound, and vibrations.

In some embodiments, the system 2040 comprises a plurality of portable diagnostic apparatuses 2010. In some embodiments, the system 2040 comprises at least 2, 5, 10, 100, 1000, 10,000 portable diagnostic apparatuses 2010. In some embodiments, the system comprises 2-50, 10-100, 50-500, or 100-1000 portable diagnostic apparatuses 2010.

In some aspects, the server 2020 is a cloud-based platform. In certain embodiments, the server 2020 is connected wirelessly to the user terminal 2030 and to the portable diagnostic apparatus 2010. In some embodiments, the server 2020 further comprises a software update module (not shown) to transmit software to the portable diagnostic apparatus 2010. This can include software containing updates to protocols for diagnostic tests, firmware updates, and other types of software updates. In some aspects, the server 2020 can send solutions to problems faced by the user in the form of remote technical support. For example, the machine operational data or the environmental data received by the server 2020 indicates that the apparatus has certain issues, the server 2020 can send information or actual software updates to address the issues.

According to another embodiment, the data module 2022 for analyzing the raw data performs one or more of the following steps:

collects raw data;

conducts analysis on the raw data to provide results; and

transmits the results to the user terminal 2030.

In some embodiments, the data module 2022 is located on Server 2020. In other embodiments, data analysis can be done on another server, computer, or in a separate system.

In some embodiments, the analysis can be the creation of a databank, statistical analysis, analyzing raw data, such as machine operational data or environmental data, to determine the cause of machine errors, creation of mathematical models, analysis on current trends, correlation data, and mapping disease prevalence to a particular location.

According to another embodiment, the data module provides one or more of the following results:

disease prevalence at different locations displayed on a map;

disease prevalence over a period of time;

severity of a disease in a particular location;

remote technical support; and

remote software update.

Additionally, correlation between environmental conditions and apparatus status can also be determined, such as analyzing whether one or more error codes occurred due to the apparatus's exposure to unusual environmental temperatures (e.g., high heat) or humidity levels (high humidity), which were measured by the environmental measuring module 2013. In another example embodiment, additional types of analyses can be done with the data, including, but not limited to, correlation between environment conditions and disease outbreak, disease relevance, trends, patterns, prevalence, and migrations.

According to another embodiment, the data module 2021 further comprises one or more access controls to the raw data and the results. In some embodiments, the access controls are selected from a password or a security code, wherein different levels of security can be achieved. Other types of access controls known to one of skill in the art could be used, including, but not limited to incorporating the access controls into another physical device, such as a mobile device, and incorporation the access control there, using technologies such as passcode, facial recognition, fingerprint identification, 2-factorial authentication, a number keypad, or a physical key. In some embodiments, the portable diagnostic apparatus 2010 transmits raw data to the server 2020 once an hour. In some embodiments, the portable diagnostic apparatus 2010 transmits raw data to the server 2020 when the portable diagnostic apparatus 2010 is not connected to an external power source.

According to another embodiment, the system 2040 comprises at least one user terminal 2030 or user interface (not shown). In some embodiments, the user terminal 2030 or interface is a computer or a mobile device. In some embodiments, the mobile device has wireless network functionality and is wirelessly connected to the server. In some embodiments, the wireless communication is done by one or more of the following wireless technologies, including, but not limited to satellite, Bluetooth, radio, Wi-Fi, wireless broadband, or cellular, such as 2G, 3G, 4G, 5G. In some embodiments, the mobile device further comprises an interface for displaying the results of the data module (e.g., a mobile application).

According to another embodiments, the system 2040 comprises a plurality of portable diagnostic apparatuses 2010, a plurality of user terminals 2030, and at least one server 2020.

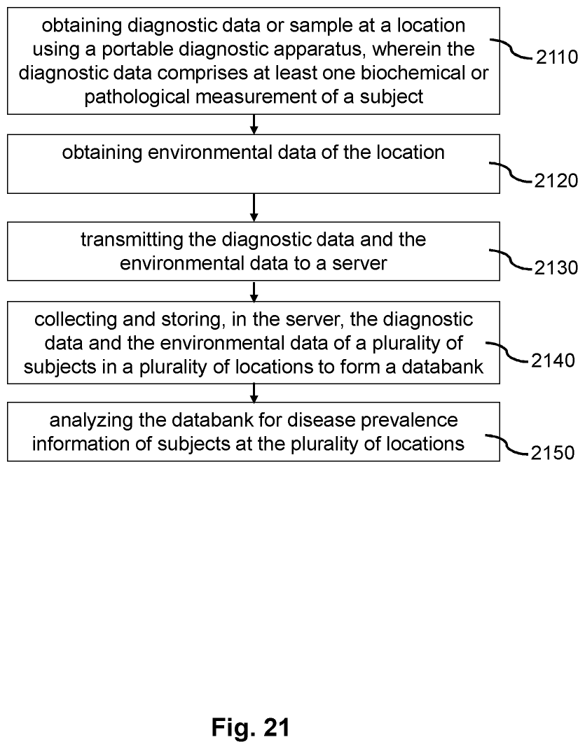

Another aspect of the invention provides a method of obtaining disease prevalence information in a location as shown in FIG. 21 and are detailed below:

Block 2110 states obtaining diagnostic data or sample at the location using a portable diagnostic apparatus, wherein the diagnostic data comprises at least one biochemical or pathological measurement of a subject.

Block 2120 states obtaining environmental data.

Block 2130 states transmitting the diagnostic data and the environmental data to a server.

Block 2140 states collecting and storing, in the server, the diagnostic data and the environmental data of a plurality of subjects in a plurality of locations to form a databank.

Block 2150 states analyzing the databank for disease prevalence or environmental information of subjects or the locations.

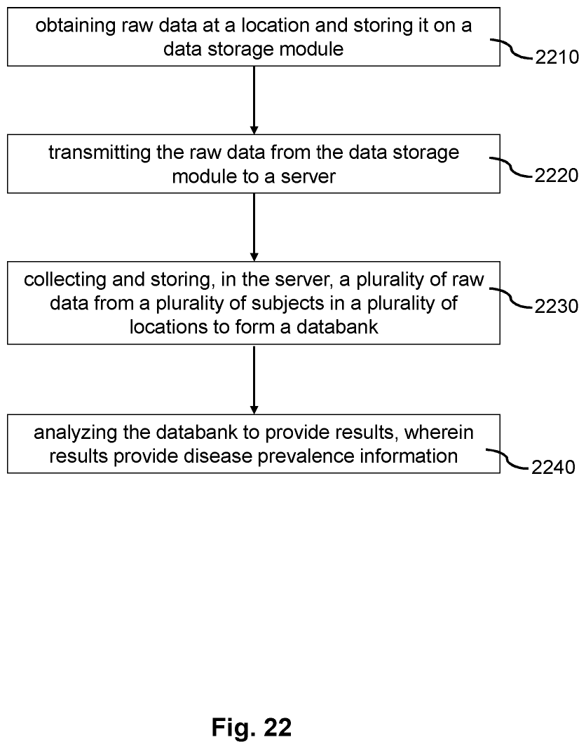

Some embodiments further comprise one or more of the following steps as shown in FIG. 22 and are detailed below:

Block 2210 states obtaining raw data at the location and storing it on a data storage module.

Block 2220 states transmitting the raw data from the data storage module to a server.

Block 2230 states collecting and storing, in the server, a plurality of raw data from a plurality of subjects in a plurality of locations to form a databank.

Block 2240 states analyzing the databank to provide results, wherein results provide disease prevalence information.

The raw data comprises one or more of the following:

Diagnostic data obtained at a location using a portable diagnostic apparatus, wherein the diagnostic data comprises at least one biochemical or pathological measurement of a subject.

Environmental data.

Apparatus data obtained from the apparatus.

In some embodiments, the portable diagnostic apparatus is an apparatus described herein.

In some embodiments, the environmental data is obtained at the location using an environmental measuring module wherein the environmental data comprises at least one environmental parameter. In other embodiments, the environmental data is obtained for a third source, such as from an environmental measuring device or from public information about the environment at the location, such as local news sources, weather observatory reports, and the internet. Examples of environmental measuring devices include, but are not limited to, devices for measuring one or more of humidity, temperature, air velocity, air pressure, light, dust, sound, and vibrations. In some embodiments, the environmental measuring devices include a device for measuring positioning data, such as GPS (global positioning system).

In some embodiments, the system grants access to the raw data, databank, and results by users according to access right.

Some embodiments further comprise the step of transmitting software from the server to the apparatus. In some embodiments, the raw data is transmitted to the server once an hour, even when the apparatus is not connected to an external power source.

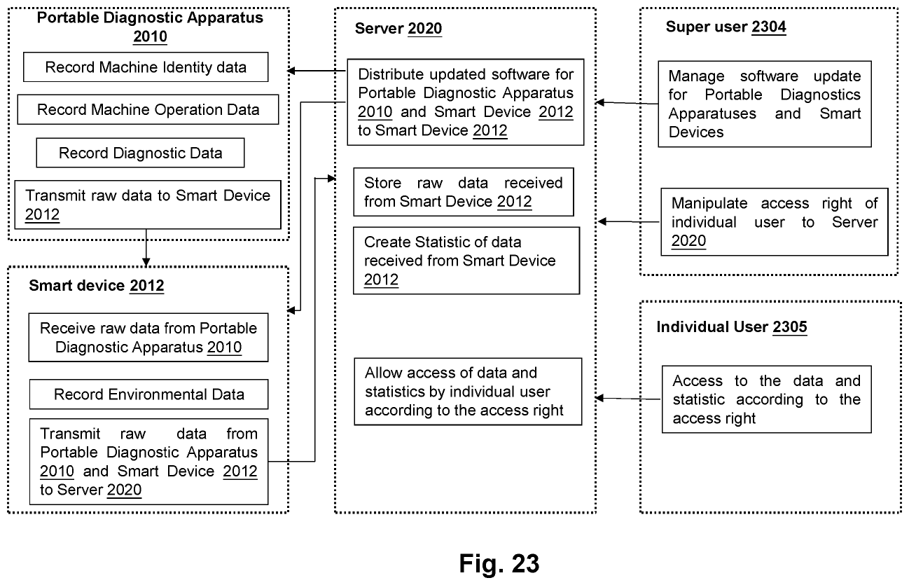

FIG. 23 is a flow chart illustrating the flow of information and data throughout the various components of a system according to an embodiment of the present invention. The system includes a portable diagnostic apparatus 2010, a smart device 2012, a server 2020, and one or more users of the system who interact with the system via user interfaces or terminals.

Portable diagnostic apparatus 2010 records raw data such as machine identity, operation status, and diagnostic data, and sends the raw data to the smart device 2012. Smart device 2012 receives the raw data, records environmental data, and transmits both the raw data and the environmental data to server 2020.

Server 2020 receives and stores raw data and environmental data received from smart device 2012. Server 2020 can also receive and store raw data directly from portable diagnostic apparatus 2010 if portable diagnostic apparatus 2010 is connected to a network. Server 2020 sends updated software to portable diagnostic apparatus 2010 via the network or via smart device 2012. Server 2020 sends updated software directly to smart device 2012 without the use of a separate network, such as Wi-Fi or cellular connection. Server 2020 controls access of data and statistics according to access right by users.

Server 2020 also analyzes the data from the smart device 2012, portable diagnostic apparatus 2010, and even a third source to conduct data analysis and create results, such as statistics, disease prevalence analyses, disease trends, and other reports.

Different types of users can access the server 2020. Super user 2304 has full control rights and can manage software updates for a plurality of apparatuses and smart devices. It can also control the access rights of individual users to the server 2020. Individual users 2305 can access the data and results according to their individual user rights.

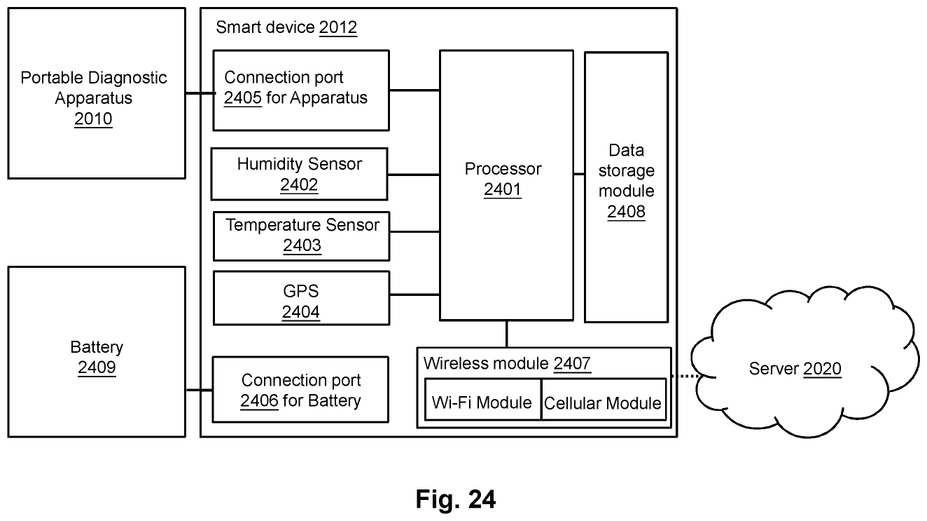

FIG. 24 is a schematic view of an assembly of smart device 2012 and units with which it interacts: an portable diagnostic apparatus 2010 and a server 2020. Smart device 2012 comprises processor 2401, data storage module 2408, humidity sensor 2402, temperature sensor 2403, GPS 2404, connection port 2405, connection port 2406, and wireless module 2407.

Processor 2401 is connected to data storage module 2408, humidity sensor 2402, temperature sensor 2403, GPS 2404, connection port 2405, and wireless module 2407. Processor 2401 collects data from humidity sensor 2402, temperature sensor 2403, and GPS 2404 and also collects raw data and machine data from the portable diagnostic apparatus 2010 via connection port 2405. The data collected by the processor 2401 are stored in data storage module 2408. Processor 2401 can directly send the data to a server 2020 via wireless module 2407 for further analysis. Wireless module 2407 consists of both a Wi-Fi module as well as a cellular module, such as 4G. In a further example embodiment, processor 2401 can also analyze all the data.

Battery 2409 is connected to Processor 2401 via connection port 2406 and provides electricity to run processor 2401 and enable wireless transmission of data to the server 2020.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the apparatus can further include at least one USB port or any other data communication means to allow the operation of common communication protocols of data transfer. The display unit is equipped in the apparatus for human interface. The display unit 450 is a high resolution color display that can be either a liquid-crystal display (LCD), Organic Light-Emitting Diode (OLED) or other kind of display. The display unit can be incorporated with a touch screen panel; therefore, it can receive command from the touch of human fingers. The display unit is optionally connected with the control unit 440. However, the way it displays, the content being displayed is made by the graphic user interface.

An exemplary microfluidic chip that can be used can be the microfluidic chip disclosed in German patent application numbers DE102010061910.8, DE102010061909.4, DE102014117976A1 and DE502007004366.4.

In yet another alternative embodiment, instead of using the at least one light beam, at least one laser beam can be used generate at least one signal for the analysis. The illumination component 1610 in this alternative embodiment emits at least one laser beam with at least one predetermined wavelength on the diagnostic portion 210. The illumination component 1610 comprises a diode laser, at least one filter and at least one dichroic mirror.

In another embodiment, the illumination component 1610 can have more than one diode laser or more than one LED.

In yet another embodiment, the camera 1621 of the optical unit 430 can be a digital high resolution camera, in which the sensor is selected from a group of Complementary metal-oxide-semiconductor (CMOS) sensor and Charge-coupled device (CCD) sensor. The megapixels of the image sensor of the digital high resolution camera are in a range of 1.0 Megapixels to 30 Megapixels.

In yet other embodiment, the portable diagnostic apparatus 400 can include multiple cartridge driver units 420, multiple cartridge receiving units 410 and multiple optical units 430 so that the multiple analyses/diagnoses can be run at the same time. While we have described a number of embodiments of this invention, it should be understood that these examples may be altered to provide other embodiments of the invention. Therefore, the scope of this invention is to be defined by the following claims rather than by the specific embodiments provided herein.

1. A portable diagnostic apparatus for detecting at least one analyte from a sample, the portable diagnostic apparatus comprising:
a cartridge receiving unit configured to receive a microfluidic cartridge therein and aligning at least part of a diagnostic portion of the microfluidic cartridge with an optical unit of said portable diagnostic apparatus; and
a cartridge driver unit comprising:
a) a microvalve controller configured to control at least some of a plurality of the microvalves of a microfluidic cartridge received by the portable diagnostic apparatus, and
b) a micropump controller configured to actuate at least some of a plurality of the micropumps of the microfluidic cartridge received by the portable diagnostic apparatus,
wherein the micropump controller and the microvalve controller are configured to cooperatively operate to actuate the flow of fluids from one or more of the plurality of the reservoirs to the diagnostic portion in a predetermined sequence.

2. The portable diagnostic apparatus of claim 1, wherein the microvalve controller comprises at least one heating element configured to apply heat energy to a heat-deformable surface of the at least one microvalve of the plurality of microvalves for opening thereof.

3. The portable diagnostic apparatus of claim 2, wherein the at least one heating element is juxtapose to at least one microvalve of the microfluidic cartridge when the cartridge is placed into the cartridge receiving unit.

4. The portable diagnostic apparatus of claim 3, wherein the heating element is an infra-red emitter.

5. The portable diagnostic apparatus of claim 1, wherein the micropump controller comprises at least one electrical connector for electrical connection with the at least one micropump of the microfluidic cartridge, wherein said at least one electrical connector is configured to provide electrical current to the at least one micropump, and the at least one electrical connector is configured so as to be mounted juxtapose the location of the at least one micropump of the microfluidic cartridge received in the cartridge receiving unit of the portable diagnostic apparatus.

6. The portable diagnostic apparatus of claim 1, wherein the optical unit comprises an illumination component and a sensor component, and wherein
a) the illumination component is configured to deliver light to a diagnostic portion of the microfluidic cartridge, and
b) the sensor component is configured to detect at least one signal generated from the diagnostic portion cause by the presence of an analyte when a microfluidic cartridge is inserted and operated at a predetermined condition.

7. The portable diagnostic apparatus of claim 6, wherein the illumination component comprises a light source having a wavelength in the range of 600 nm to 650 nm and the at least one data signal is a fluorescent signal.

8. The portable diagnostic apparatus of claim 1, further comprising a control unit configured to perform one or more of the following:
a) provide a predetermined control sequence to the cartridge driver unit for directing at least one fluid movement within the microfluidic cartridge;
b) provide a predetermined condition to the optical unit for performing a quantitative and/or qualitative analysis of the analyte;
c) store a data signal obtained from the optical unit; and
d) control and monitor an operation of the apparatus.

9. Canceled

10. The portable diagnostic apparatus of claim 1, further comprising:
a housing for anchoring the cartridge receiving unit, the cartridge driver unit and the optical unit therein,
wherein the cartridge receiving unit further comprises a rail component and a tray, wherein
the rail component comprises a pair of slidable rails, and
the tray is configured to receive the microfluidic cartridge and is anchored on the pair of rails, and
wherein the rails may slide the tray in and out of the housing such that the microfluidic cartridge may be inserted into the housing.

11-13. (canceled)

14. The portable diagnostic apparatus of claim 8, further comprising:
an identification unit to read the identity of the microfluidic cartridge and transmit a corresponding identity signal to the control unit.

15. The portable diagnostic apparatus of claim 14, further comprising:
a switch to trigger the identification unit to read the identity of the microfluidic cartridge when the microfluidic cartridge is positioned in a designated area.

16. Canceled.

17. The portable diagnostic apparatus of claim 8, further comprising:
a user interface unit configured to display the quantitative and/or qualitative analysis of the analyte, wherein the user interface unit is connected to the control unit.

18. Canceled.

19. The portable diagnostic apparatus of claim 1, wherein the cartridge receiving unit comprises a rail component and a tray, wherein
the rail component comprises a cavity for slidably receiving the tray, and
the tray comprises a cartridge chamber for receiving the microfluidic cartridge such that the microfluidic cartridge is positioned at the designated area.

20. The portable diagnostic apparatus of claim 1, further comprising:
a removable device, wherein the removable device comprises:
a) an environmental measuring module for acquiring environmental data, selected from positioning data, humidity, temperature, and time, wherein the environmental data comprises at least one environmental parameter at the location;
b) a data storage module for storing raw data, wherein the raw data comprises one or more of environmental data and diagnostic data; and
c) a transmitter for transmitting the raw data to a remote server.

21-23. (canceled)

24. A method of detecting at least one analyte from a sample using the portable diagnostic apparatus of claim 1 wherein the method comprises:
a) loading the sample into the microfluidic cartridge;
b) directing the sample and at least one reagent from the microfluidic portion to the diagnostic portion within the microfluidic cartridge in a predetermined sequence by opening at least one microvalve and actuating at least one micropump in the microfluidic cartridge;
c) providing a predetermined condition to the diagnostic portion of the microfluidic cartridge to generate at least one signal;
d) detecting the at least one data signal and collecting diagnostic data using an optical sensor; and
e) analyzing the diagnostic data to determine the presence of the analyte quantitatively and/or qualitatively;
optionally, the method further comprising:
a) reading the identity of the microfluidic cartridge;
b) providing a predetermined sequence to the cartridge driver unit and a predetermined condition to the optical unit based on the identity of the microfluidic cartridge.

25. (canceled)

26. (canceled)

27. A system for managing a network of portable diagnostic apparatuses, comprising:
at least one portable diagnostic apparatus of claim 20;
at least one user terminal; and
a server comprising:
a data module for collecting and storing raw data, wherein the raw data comprises one or more of the following:
a) diagnostic data obtained at a location using a portable diagnostic apparatus, wherein the diagnostic data comprises at least one biochemical or pathological measurement of a subject,
b) environmental data obtained at the location using an environmental measuring module, wherein the environmental data comprises at least one environmental parameter,
c) apparatus data obtained from the portable diagnostic apparatus, and
d) a data module for analyzing the raw data
wherein the server is configured for communication with the user terminal and to the portable diagnostic apparatus.

28. (canceled)

29. The system of claim 27, wherein the data module is configured to perform one or more of the following:
a) collecting raw data;
b) analyzing the raw data to provide results; and
c) transmitting results to the user terminal;
and provides one or more of the following results:
a) disease prevalence at different locations displayed on a map;
b) disease prevalence over a period of time;
c) severity of a disease in a particular location; and
d) correlation between environmental conditions and apparatus status.

30-34. (canceled)

35. A method of using the system of claim 29, comprising:
obtaining raw data at the location and storing it on a data storage module;
transmitting the raw data from the data storage module to a server;
collecting and storing, in the server, a plurality of raw data from a plurality of portable diagnostic apparatuses to form a databank; and
analyzing the databank to provide results;
wherein the raw data comprises one or more of the following:
a) diagnostic data obtained at a location using a portable diagnostic apparatus, wherein the diagnostic data comprises at least one biochemical or pathological measurement of a subject;
b) environmental data obtained at the location using an environmental measuring module, wherein the environmental data comprises at least one environmental parameter; and
c) apparatus data obtained from the portable diagnostic apparatus.

36. The method of claim 35, wherein the raw data is diagnostic data obtained at a location using a portable diagnostic apparatus, wherein the diagnostic data comprises at least one biochemical or pathological measurement of a subject and location data; and the results provide disease prevalence information.

37. The method of claim 35, wherein the raw data is one or more of temperature, humidity, time, positioning data, and apparatus data; and the results provide information associated with performance of the portable diagnostic apparatus.
